(19) United States
(12) Patent Application Publication
(10) Pub. No.: US 2023/0119060 A1
(43) Pub. Date: Apr. 20, 2023

(54) PHARMACEUTICAL COMPOSITION FOR PREVENTING OR TREATING CANCER CONTAINING, AS ACTIVE INGREDIENT, COMPLEX OF BIGUANIDE-BASED COMPOUND AND FLAVONE, HYDROXYFLAVONE, FLAVANONE, FLAVONE DERIVATIVE, HYDROXYFLAVONE DERIVATIVE, OR FLAVANONE DERIVATIVE

(71) Applicant: FRONTBIO INC., Chuncheon-si, Gangwon-do (KR)

(72) Inventors: Jae Yong LEE, Chuncheon-si, Gangwon-do (KR); Hong Won SUH, Chuncheon-si, Gangwon-do (KR); Soon Sung LIM, Chuncheon-si, Gangwon-do (KR); Madhuri Rame-Shwar SHENDE, Chuncheon-si, Gangwon-do (KR)

(21) Appl. No.: 17/905,541

(22) PCT Filed: Aug. 20, 2020
(86) PCT No.: PCT/KR2020/011115

§ 371 Date: Sep. 2, 2022

(30) Foreign Application Priority Data
Mar. 5, 2020 (KR) 10-2020-0027715

(51) Int. Cl. A61K 31/155; A61K 31/352; A61K 31/7048; A61P 35/00; A61P 1/00; A61P 11/00; A61P 13/08; A61P 1/18

(52) U.S. Cl. CPC A61K 31/155; A61K 31/352; A61K 31/7048; A61P 35/00; A61P 1/00; A61P 11/00; A61P 13/08; A61P 1/18

(57) ABSTRACT

The present invention relates to a pharmaceutical composition for prevention or treatment of cancer, containing a complex, mixed or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof as an active ingredient. It has been confirmed that a significantly higher synergistic anticancer activity is exhibited in a case where the complex, mixed or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof is administered compared to a case where the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof are each administered singly. Consequently, the pharmaceutical composition containing a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof in a complex, mixed or combined manner according to the present invention can be usefully utilized for the prevention or treatment of cancer.

### TECHNICAL FIELD

The present invention relates to a pharmaceutical composition for prevention or treatment of cancer, containing a first component including a biguanide-based compound or a pharmaceutically acceptable salt thereof and a second component including a flavone, a hydroxyflavone, a flavanone, a flavone derivative, a hydroxyflavone derivative, a flavanone derivative or a pharmaceutically acceptable salt thereof as active ingredients of a complex, mixed or combined preparation, more particularly to a pharmaceutical composition for prevention or treatment of cancer in which the blending ratio of a first component including a biguanide-based compound or a pharmaceutically acceptable salt thereof to a second component including a flavone, a hydroxyflavone, a flavanone, a flavone derivative, a hydroxyflavone derivative, a flavanone derivative or a pharmaceutically acceptable salt thereof is from 1:0.0000001 to 1:10 parts by weight.

### BACKGROUND ART

Cancer is a disease caused by the uncontrolled growth of abnormal cells that may spread in contact with tissues or other parts of the body, and cancer cells may form solid tumors in which the cancer cells cluster together, or may exist as dispersed cells as in leukemia. Normal cells differentiate until mature and then replace damaged or dead cells as needed, but cancer cells constantly differentiate to eventually push out nearby cells and spread to other parts, which is called malignancy. Malignant tumor cells metastasize to other parts of the body through the bloodstream or lymphatic system, where they proliferate and form new tumors.

Despite the development of various treatment methods, cancer still seriously threatens human health worldwide. Current major cancer treatment methods include surgery, radiation therapy, hormone therapy, and chemotherapy, and among these, chemotherapy is a method for treating cancer directly or relieving symptoms using one or more anticancer drugs.

Traditional chemotherapeutic agents exhibit cytotoxicity to cancer cells by interfering with the division and metabolism of cancer cell or suppressing the biosynthesis of nucleic acids or proteins. However, these chemotherapeutic agents have a problem that cancer cells have resistance to an anticancer drug and a problem of causing serious side effects that an anticancer drug exhibits toxicity to normal tissues. In particular, substances used as existing anticancer drugs not only affect cancer cells but are also toxic to normal cells and thus cause various side effects in many cases. Therefore, there is a need for an anticancer drug that is not toxic to normal cells yet exhibits excellent selective toxicity only to cancer cells and exhibits excellent anticancer activity.

Targeted anticancer drugs are one of those that have been developed to solve the side effects and problems of chemotherapeutic agents used in conventional chemotherapy. Since targeted anticancer drugs attack specific targets expressed only in cancer cells, it has been expected that the therapeutic effect can be increased while side effects are diminished compared to conventional chemotherapeutic agents. For example, imatinib (Gleevec), which attacks BCR-ABL, a gene specifically expressed in chronic myeloid leukemia, gefitinib, erlotinib, and afatinib, which are used to treat lung cancer with mutations in the epidermal growth factor receptor (EGFR), crizotinib used to treat ALK-mutated lung cancer, trastuzumab used to treat HER2-positive breast and gastric cancer, and rituximab used to treat CD20-positive lymphoma are representative targeted anticancer drugs. However, in the case of targeted anticancer drugs, there is a limitation in that the therapeutic effect is obtained only when a specific therapeutic target is expressed. In other words, EGFR inhibitors are effective only for lung cancer with EGFR mutations but are not effective for ALK-positive lung cancer. Targeted therapeutic agents also have a problem in that resistance develops after a certain period of time. This is because cancer cells find another signaling pathway and continue cell proliferation even if a targeted therapeutic agent blocks one cancer cell proliferation signal.

Immuno-oncology agents are intended to solve the problems of these chemotherapeutic agents and targeted anticancer drugs. Immune cells attack when abnormal cells appear, but cancer cells attack these immune cells and weaken the function of immune cells to create an environment where cancer cells thrive. Immuno-oncology agents help immune cells to kill cancer cells by blocking the pathway by which cancer cells attack the immune cells or strengthening the immune cells themselves. Keytruda (ingredient name: pembrolizumab), which is from a multinational pharmaceutical company MSD, is an immune checkpoint inhibitor that blocks the point where cancer cells attack immune cells, and Immuncell-LC, which is a immunotherapy medicine from Green Cross Cell, is a therapeutic agent for liver cancer. Immunotherapy medicine is now a newly launched means and is still in the development stage.

Meanwhile, metabolism-modulating anticancer drugs utilize the difference in the metabolic process between cancer cells and normal cells to make normal cells grow and suppress the proliferation of cancer cells by metabolic components that cancer cells cannot use. Since the metabolic means of cancer cells does not change, metabolism-modulating anticancer drugs are less affected by genetic mutations and thus have fewer problems of drug resistance occurring in the existing cancer treatment process. Metabolism-modulating anticancer drugs for leukemia were approved for use in the United States in 2017, and additional approvals for other cancers such as breast cancer are expected in 2018.

Metformin, phenformin, buformin, and biguanide are all biguanide-based drugs, and are still widely used as medications for type 2 diabetes, which inhibit the production of glucose in the liver and promote the use of glucose in peripheral blood vessels. Metformin, phenformin, buformin, or biguanide activates AMPK (AMP-activated protein kinase), a key enzyme in metabolic regulation, to inhibit the synthesis of protein, fat lipid, and glycogen and promote the degradation thereof, and inhibits the production of insulin, IGF1, leptin, and adiponectin. Meanwhile, activated AMPK suppresses cell regeneration, and thus inhibits metabolism of cancer cells and inhibits cell division. AMPK activation suppresses the proliferation of cancer cells by directly inhibiting mTOR (mammalian target of rapamycin) and eventually inhibiting protein synthesis. In particular, it has been reported that metformin suppresses the growth of cancer cells by inhibiting the expression of angiogenesis promoters. Due to this anticancer mechanism, metformin and phenformin have been used in clinical trials of various kinds of cancers alone or in combination with other anticancer drugs, but the therapeutic effect varies, and metformin and phenformin have not yet been approved as anticancer drugs because of several problems.

Accordingly, the present inventors have studied to develop a combination of substances having a superior anticancer effect, as a result, revealed the fact that a combination of a biguanide-based compound with a flavone, hydroxyflavone, or flavanone-based compound shows a remarkable synergism in the anticancer effect, and thus applied for a new complex, mixed or combined anticancer drug.

### CITATION LIST

### Patent Literature

### [Patent Literature 1]

US 2014-0113930 A1 (2014.4.24.)

### Non Patent Literature

### [Non Patent Literature 1]

Yip, K. W.; Reed, J. C. BCL-2 family proteins and cancer. Oncogene 2008, 77, 6398-6406.

### [Non Patent Literature 2]

J. Xu, and W. Mao Overview of Research and Development for Anticancer Drugs, Journal of Cancer Therapy, 2016, 7, 762-772.

### [Non Patent Literature 3]

Dallaglio K, Bruno A, Cantelmo A R, Esposito A R, Ruggiero L, Orecchioni S, et al. Paradoxic effects of metformin on endothelial cells and angiogenesis. Carcinogenesis 2014; 35:1055-66.

### [Non Patent Literature 4]

Ying-Wei Li, Jian Xu, Guo-Yuan Zhu, Zhu-Juan Huang, Yan Lu, Xian-Qian Li, Neng Wang, Feng-Xue Zhang. Apigenin suppresses the stem cell-like properties of triple-negative breast cancer cells by inhibiting YAP/TAZ activity. Cell Death Discov. 2018 Nov. 20; 4:105

### [Non Patent Literature 5]

Masayuki YOSHIKAWA, Toshiaki UEMURA, Hiroshi SHIMODA, Akinobu KISHI, Yuzo KAWAHARA, Hisashi MATSUDA. Medicinal Foodstuffs. XVIII. Phytoestrogens from the Aerial Part of Petroselinum crispum MILL. (PARSLEY) and Structures of 60-Acetylapiin and a New Monoterpene Glycoside. Petroside Chem Pharm Bull (Tokyo). 2000 July; 48(7):1039-44.

### SUMMARY OF INVENTION

### Technical Problem

Substances used as existing anticancer drugs not only affect cancer cells but are also toxic to normal cells, for example, rapidly dividing normal cells, such as skin, mucous membranes, and blood cells, and thus cause various side effects such as hair loss, diarrhea, and leukopenia in many cases. Unlike normal cells, the expression of antiapoptotic proteins such as BCL-2 is increased or the expression of proapoptotic proteins such as BAX is suppressed and apoptosis is often lacked in many cancer cells. In cancer cells, the expression of caspases may be low or mutations in the caspase gene may occur. In some cases, apoptosis may be inhibited as mitochondrial outer membrane permeabilization (MOMP) is inhibited in cancer cells. As described above, since apoptosis does not occur in many cancer cells, there is a problem that the therapeutic effect of many anticancer drugs that induce apoptosis is not obtained.

Hence, there is an urgent demand for an anticancer drug that exhibits excellent anticancer activity while not being toxic to normal cells but exhibiting excellent selective toxicity only to cancer cells.

### Solution to Problem

In the present invention, it has been found out that a pharmaceutical composition for prevention or treatment of cancer as a complex, mixed or combined preparation for use in the prevention or treatment of cancer, which contains a first component including a biguanide-based compound or a pharmaceutically acceptable salt thereof and a second component including a flavone, a hydroxyflavone, a flavanone, a flavone derivative, a hydroxyflavone derivative, a flavanone derivative or a pharmaceutically acceptable salt thereof as active ingredients of the complex, mixed or combined preparation, does not exhibit toxicity to normal cells but exhibits cancer cell-specific and synergistic anticancer activity, and the above problems have been solved by providing the pharmaceutical composition containing the first component and the second component as active ingredients of the complex, mixed or combined preparation.

In an aspect of the present invention, the biguanide-based compound may be selected from the group consisting of metformin, phenformin, buformin and biguanide.

In an aspect of the present invention, the flavone, hydroxyflavone, flavanone, flavone derivative, hydroxyflavone derivative or flavanone derivative may be selected from the group consisting of
2′-hydroxyflavone,
3-hydroxyflavone (flavonol),
3′-hydroxyflavone,
4′-hydroxyflavone,
5-hydroxyflavone (primuliten),
6-hydroxyflavone,
7-hydroxyflavone,
8-hydroxyflavone,
3′,4′-dihydroxyflavone,
3,6-dihydroxyflavone,
3,7-dihydroxyflavone (resogalangin),
4′,7-dihydroxyflavone,
5,7-dihydroxyflavone (chrysin),
7-O-acetyl chrysin (monoacetyl chrysin),
5,7-di-O-methoxy chrysin (dimethyl chrysin),
5,7-di-O-acetyl chrysin (diacetyl chrysin),
6,7-dihydroxyflavone,
7,4′-dihydroxyflavone,
7,8-dihydroxyflavone,
3,5,7-trihydroxyflavone (galangin),
3,7,4′-trihydroxyflavone (resokaempferol),
4′,5,7-trihydroxyflavanone (naringenin),
5,3′,4′-trihydroxyflavone,
5,6,7-trihydroxyflavone (baicalein),
5,7,2′-trihydroxyflavone,
5,7,4′-trihydroxyflavone (apigenin),
5,7,8-trihydroxyflavone (norwogonin),
7,3′,4′-trihydroxyflavone,
7,8,3′-trihydroxyflavone,
7,8,4′-trihydroxyflavone,
4′,5,7-triacetoxy flavone (apigenin triacetate),
5-hydroxy-4′,7-dimethoxyflavone,
5,7-dimethoxy-4′-hydroxyflavone,
5,4′-dimethoxy-7-hydroxyflavone,
3′,4′,5,7-tetrahydroxyflavone (luteolin),
3,4′,5,7-tetrahydroxyflavone (kaempferol),
5,6,7,4′-tetrahydroxyflavone (scutellarein),
4′,5,6,7,8-pentamethoxyflavone (tangeretin),
5,6,7,3′,4′-pentamethoxyflavone (sinensetin),
5,7,8,3′,4′-pentamethoxyflavone (isosinensetin),
3,3′,4′,5,6,7-hexahydroxyflavone (quercetagetin),
3′,4′,5,6,7,8-hexamethoxyflavone (nobiletin),
4′,5,7-trihydroxy-3′-methoxyflavone (chrysoeriol),
5,7,3′-trihydroxy-4′-methoxyflavone (diosmetin),
4′,5,7-trihydroxy-6-methoxyflavone (hispidulin),
5,7,4′-trihydroxy-3,6,3′-trimethoxyflavone
(jaceidin),
3′,4′,7-trihydroxy-6-methoxyflavone (nepetin),
3,5,7,3′,4′-pentahydroxy-6-methoxyflavone (patuletin),
3,4′,5,7-tetrahydroxy-3′,6-dimethoxyflavone (spinacetin),
5,7,4′-trihydroxy-3′,5′-dimethoxyflavone (tricin),
7-O-beta-D-apiofuranosyl-1,2-beta-D-glucosyl-5,7,4′-trihydroxyflavone (apiin),
7-O-beta-D-glucosyl-5,7,4′-trihydroxyflavone (apigetrin),
5,7,3′,4′-flavon-3-ol (quercetin),
7,3′,4′-flavon-3-ol (fisetin),
4′,5-dihydroxy-7-methoxyflavone (genkwanin),
4′,5,7-trihydroxyflavanone-7-rhamnoglucoside (naringin),
5-hydroxy-2-(4-hydroxyphenyl)-4-oxo-4H-chromen-7-yl-2-O-(alpha-L-rhamnopyranosyl)-beta-D-glucopyranoside (rhoifoloside) and
8alpha-L-arabinopyranosyl-6beta-D-glucopyranosyl-5,7-dihydroxy-2-(4-hydroxyphenyl)-4H-1-benzopyran-4-one (schaftoside).

In an aspect of the present invention, a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof may be blended at a weight ratio of 1:0.0000001 to 10.

In an aspect of the present invention, the cancer may be selected from the group consisting of (A) breast cancers, including (1) ductal carcinoma, including ductal carcinoma in situ (DCIS) (comedocarcinoma, cribriform, papillary, micropapillary), invasive ductal carcinoma (IDC), ductal carcinoma, mucinous (colloidal) carcinoma, papillary carcinoma, metaplastic carcinoma and inflammatory carcinoma; (2) lobular carcinomas, including lobular carcinoma in situ (LCIS) and invasive lobular carcinoma; and (3) Paget's disease of the nipple; (B) cancers of the female reproductive system, including (1) cancers of the cervix, including cervical intraepithelial neoplasia (grade I), cervical intraepithelial neoplasia (grade II), cervical intraepithelial neoplasia (grade III) (squamous cell carcinoma in situ), keratinizing squamous cell carcinoma, nonkeratinizing squamous cell carcinoma, verrucous carcinoma, adenocarcinoma in situ, adenocarcinoma in situ, endometrial type carcinoma, endometrioid adenocarcinoma, clear cell adenocarcinoma, adenoepithelioma, adenoid cystic carcinoma, small cell carcinoma and undifferentiated carcinoma; (2) cancers of the uterine body, including endometrioid carcinoma, adenocarcinoma, adenoacanthoma (adenocarcinoma with squamous metaplasia), adenoepithelioma (mixed adenocarcinoma and squamous cell carcinoma), mucinous adenocarcinoma, serous adenocarcinoma, clear cell adenocarcinoma, squamous cell adenocarcinoma and undifferentiated adenocarcinoma; (3) cancers of the ovary, including serous cystadenoma, serous cystadenoma, mucinous cystadenoma, mucinous cystadenoma, endometrioid tumor, endometrioid adenocarcinoma, clear cell tumor, clear cell cystadenoma and unclassified tumors; (4) cancers of the vagina, including squamous cell carcinoma and adenocarcinoma; and (5) vulvar cancers, including vulvar intraepithelial neoplasia (grade I), vulvar intraepithelial neoplasia (grade II), vulvar intraepithelial neoplasia (grade III) (squamous cell carcinoma in situ); squamous cell carcinoma, verrucous carcinoma, Paget's disease of the vulva, adenocarcinoma (NOS); basal cell carcinoma (NOS) and Bartholin gland carcinoma; (C) cancers of the male reproductive system, including (1) cancer of the penis, including squamous cell carcinoma; (2) cancers of the prostate, including adenocarcinomas, sarcomas, and transitional cell carcinomas of the prostate; and (3) cancers of the testes, including seminoma tumor, non-seminoma tumor, teratomas, embryonic carcinomas, yolk sac tumor and choriocarcinoma; (D) cancers of the heart system, including sarcomas (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma and squamous cell carcinoma of the pharynx; (F) cancers of the lung, including squamous cell carcinoma (epidermoid carcinoma), a variant of squamous cell carcinoma, spindle cell carcinoma, small cell carcinoma, carcinoma of other cells, carcinoma of the intermediate cell type, complex oat cell carcinoma, adenocarcinoma, acinar adenocarcinoma, papillary adenocarcinoma, bronchoalveolar carcinoma, mucin-producing solid carcinoma, giant cell carcinoma, giant cell carcinoma, clear cell carcinoma and sarcoma; (G) cancers of the gastrointestinal tract, including (1) cancers of the ampulla of vater, including primary adenocarcinoma, carcinoid tumor and lymphoma; (2) cancers of the anal canal, including adenocarcinoma, squamous cell carcinoma and melanoma; (3) cancers of the extrahepatic bile duct, including carcinoma in situ, adenocarcinoma, papillary adenocarcinoma, adenocarcinoma, intestinal type, mucinous adenocarcinoma, clear cell adenocarcinoma, signet ring cell carcinoma, adenoepithelioma, squamous cell carcinoma, small cell (oat cell) carcinoma, undifferentiated carcinoma, carcinoma (NOS), sarcoma and carcinoid tumor; (4) cancers of the colon and rectum, including adenocarcinoma in situ, adenocarcinoma, mucinous adenocarcinoma (colloidal type; >50% mucinous carcinoma), signet ring cell carcinoma (greater than 50% of signet ring cells), squamous cell (epidermoid) carcinoma, adenoepithelioma, small cell (oat cell) carcinoma, undifferentiated carcinoma, carcinoma (NOS), sarcoma, lymphoma and carcinoid tumor; (5) cancers of the esophagus, including squamous cell carcinoma, adenocarcinoma, leiomyosarcoma and lymphoma; (6) cancers of the gallbladder, including adenocarcinoma, adenocarcinoma, bowel type, adenoepithelioma, carcinoma in situ, carcinoma (NOS), clear cell adenocarcinoma, mucinous adenocarcinoma, papillary adenocarcinoma, signet ring cell carcinoma, small cell (oat cell) carcinoma, squamous cell carcinoma and undifferentiated carcinoma; (7) cancers of the lips and oral cavity, including squamous cell carcinoma; (8) cancers of the liver, including liver cancer (hepatocellular carcinoma), cholangiocarcinoma, hepatoblastoma, hemangiosarcoma, hepatocellular adenoma and hemangioma; (9) cancers of the exocrine pancreas, including ductal cell carcinoma, giant cell carcinoma multiforme, giant cell carcinoma, osteoclastoid type, adenocarcinoma, adenoepithelioma, mucinous (colloidal) carcinoma, cystadenoma, acinar cell carcinoma, papillary carcinoma, small cell (oat cell) carcinoma, mixed cell type, carcinoma (NOS), undifferentiated carcinoma, endocrine cell tumor arising from islet cells of Langerhans and carcinoid tumor; (10) cancers of the salivary glands, including acinar (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well-differentiated or low grade) and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenoepithelioma, squamous cell carcinoma, small cell carcinoma, undifferentiated carcinoma, lymphoma, sarcoma and carcinoid tumor; and (12) cancers of the small intestine, including adenocarcinoma, lymphoma, carcinoid tumor, Kaposi's sarcoma, leiomyoma, hemangioma, lipoma, neurofibromatosis and fibroma; (H) cancers of the urinary system, including (1) cancers of the kidney, including renal cell carcinoma, carcinoma of the Bellini collecting duct, adenocarcinoma, papillary carcinoma, tubular carcinoma, granular cell tumor, clear cell carcinoma (adenocarcinoma of the kidney), sarcoma of the kidney and nephroblastoma; (2) cancers of the renal pelvis and ureter, including transitional cell carcinoma, papillary transitional cell carcinoma, squamous cell carcinoma and adenocarcinoma; (3) cancers of the urethra, including transitional cell carcinoma, squamous cell carcinoma and adenocarcinoma; and (4) cancers of the bladder, including carcinoma in situ, transitional urothelial cell carcinoma, papillary transitional cell carcinoma, squamous cell carcinoma, adenocarcinoma, and undifferentiated carcinoma; and (I) cancers of muscles, bones and soft tissues, including (1) cancers of the bone, including (a) osteogenesis: osteosarcoma; (b) chondrogenesis: chondrosarcoma and mesenchymal chondrosarcoma; (c) giant cell tumor, malignant; (d) Ewing's sarcoma; (e) vascular tumors: hemangioendothelioma, hemangiopericytoma and hemangiosarcoma; (f) connective tissue tumors: fibrosarcoma, liposarcoma, malignant mesenchymoma and undifferentiated sarcoma; and (g) other tumors: chordoma and adamantinoma of the long bones; (2) cancers of soft tissues, including alveolar soft part sarcoma, angiosarcoma, epithelioid sarcoma, extraosseous chondrosarcoma, fibrosarcoma, leiomyosarcoma, liposarcoma, malignant fibrous histiocytoma, malignant hemangiopericytoma, malignant mesenchymoma, malignant Schwannoma, rhabdomyosarcoma, synovial sarcoma and sarcoma (NOS); (3) cancers of the nervous system, including cancers of the skull (osteoma, hemangioma, granuloma, xanthoma, and osteitis deformans), cancers of the meninges (meningioma, meningiosarcoma, and gliomatosis), cancers of the brain (astrocytoma, meduloblastoma, glioma, ependymal glioma, germinoma (pineal tumor), glioblastoma multiforme, oligodendrocytoma, schwannoma, retinoblastoma, and congenital tumor), and cancers of the spinal cord (neurofibromatosis, meningioma, glioma, sarcoma); (4) hematologic malignancies, including myeloid leukemia (acute and chronic), acute lymphoblastic leukemia, chronic lymphocytic leukemia, myeloproliferative disease, multiple myeloma; myelodysplastic syndrome, Hodgkin's disease and non-Hodgkin's lymphoma (malignant lymphoma); (5) cancers of the endocrine system, including (a) cancers of the thyroid gland, including papillary carcinoma (including those of the follicular region), follicular carcinoma, medullary carcinoma and undifferentiated (anaplastic) carcinoma; and (b) neuroblastomas, including sympathicogonioma, sympathoblastoma, malignant ganglioneuroma, ganglioneuroblastoma and ganglioneuroma; (6) cancers of the skin, including squamous cell carcinoma, spindle cell squamous cell carcinoma, basal cell carcinoma, adenocarcinoma arising from sweat glands or sebaceous glands, and malignant melanoma; and (7) cancers of the eye, including (a) cancer of the conjunctiva, including carcinoma of the conjunctiva; (b) cancers of the eyelid, including basal cell carcinoma, squamous cell carcinoma, melanoma of the eyelid and sebaceous cell carcinoma; (c) cancers of the lacrimal gland, including adenocarcinoma, adenoid cystic carcinoma, carcinoma in pleomorphic adenoma, mucoepidermoid carcinoma and squamous cell carcinoma; (d) cancers of the uvea, including spindle cell melanoma, mixed cell melanoma and epithelioid cell melanoma; (e) cancers of the orbit, including sarcoma of the orbit, soft tissue tumor, and sarcoma of the bone; and (f) retinoblastoma.

In an aspect of the present invention, the pharmaceutical composition may be formulated into a formulation selected from the group consisting of a tablet, a capsule, an injections, a troche, a powder, a granule, a solution, a suspension, an oral solution, an emulsion, a syrup, a suppository, a vaginal tablet and a pill, but is not limited thereto.

In another aspect of the present invention, there is provided a kit for prevention or treatment of cancer, including a complex, mixed or combined preparation of a biguanide-based compound or a pharmaceutically acceptable salt thereof and a flavone, a hydroxyflavone, a flavanone, a flavone derivative, a hydroxyflavone derivative, a flavanone derivative or a pharmaceutically acceptable salt thereof.

### Advantageous Effects of Invention

The anticancer effect is weak when the biguanide-based compound or a pharmaceutically acceptable salt thereof and the flavone, hydroxyflavone, flavanone, flavone derivative, hydroxyflavone derivative, flavanone derivative or a pharmaceutically acceptable salt thereof according to the present invention are each subjected to treatment singly, but a significantly high synergistic anticancer effect is exerted in diverse carcinomas when these are subjected to treatment in a complex, mixed or combined manner, and thus a pharmaceutical composition containing these as active ingredients of a complex, mixed or combined preparation can be usefully utilized for the prevention or treatment of cancer. In addition, the pharmaceutical composition does not exhibit toxicity to normal cells at an effective concentration, and thus can afford an anticancer drug having an excellent anticancer effect and significantly diminished side effects.

### BRIEF DESCRIPTION OF DRAWINGS

FIG. 1A is a diagram illustrating the degree of growth inhibition (% growth inhibition) when MCF-7 breast cancer cells are treated with 5 mM metformin; 0.01, 0.1, 1, or 10 μM 5,7,4′-trihydroxyflavone (apigenin); or 0.01, 0.1, 1, or 10 μM 5,7,4′-trihydroxyflavone+5 mM metformin for 24, 48, or 72 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours; and 4: the degree (percentage) of growth inhibition of cancer cells when treated with 5 mM metformin and 0.01, 0.1, 1, or 10 μM 5,7,4′-trihydroxyflavone in combination for 72 hours.

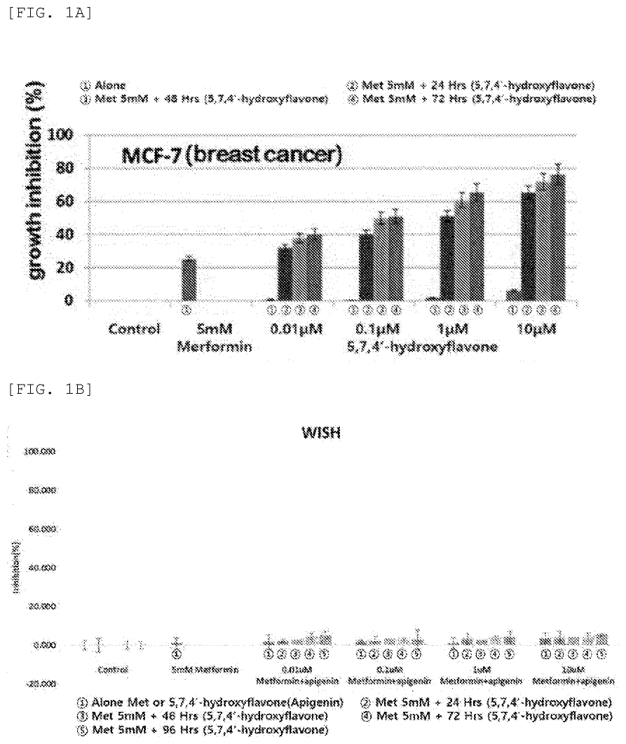

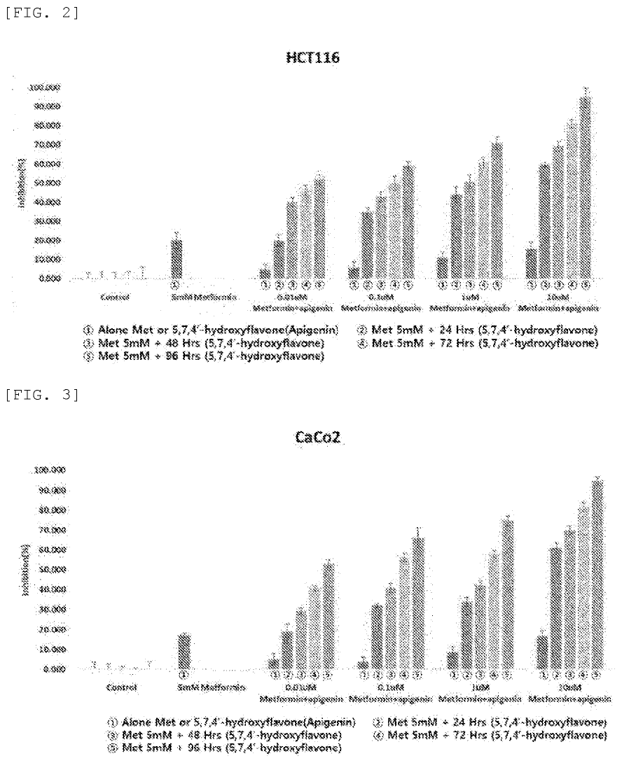

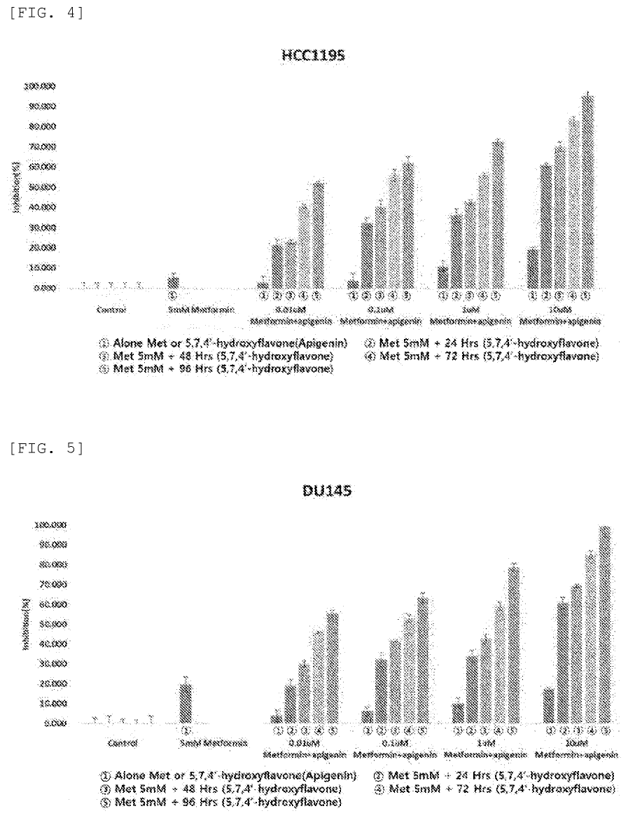

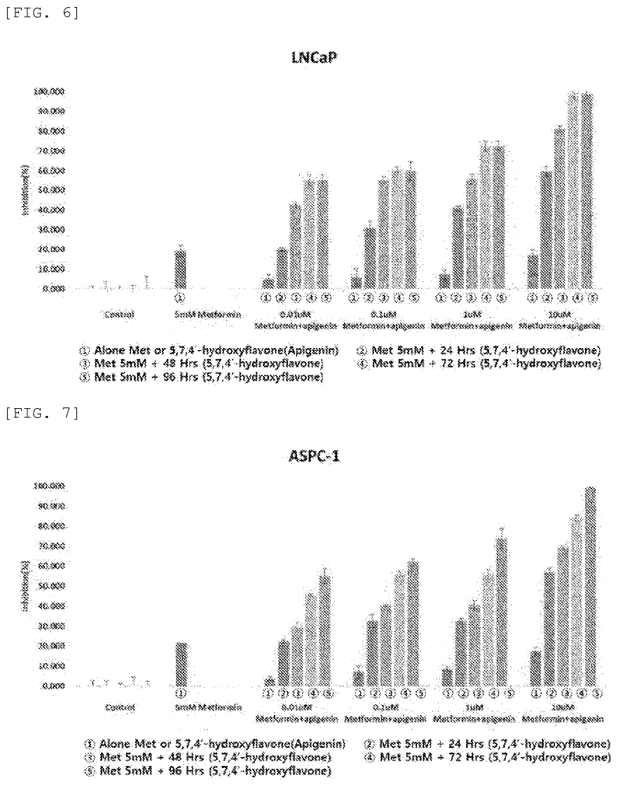

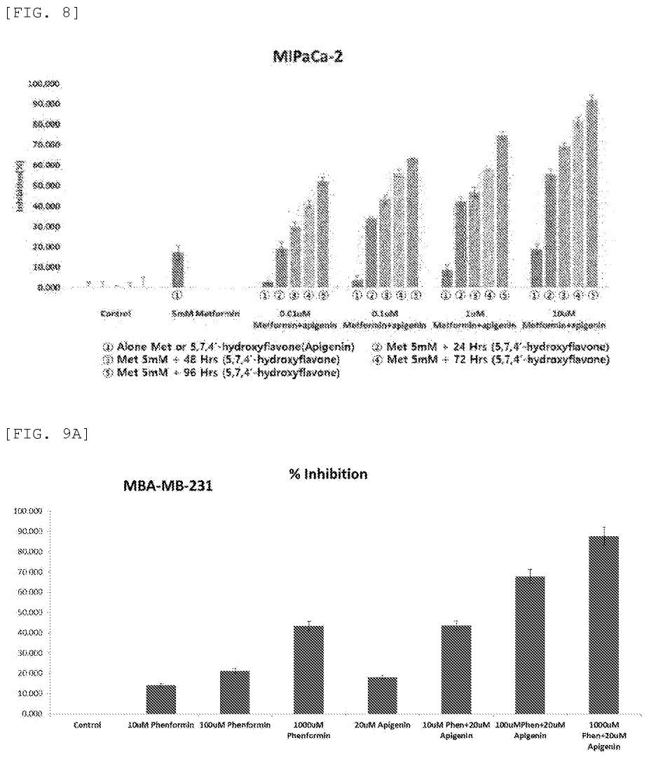

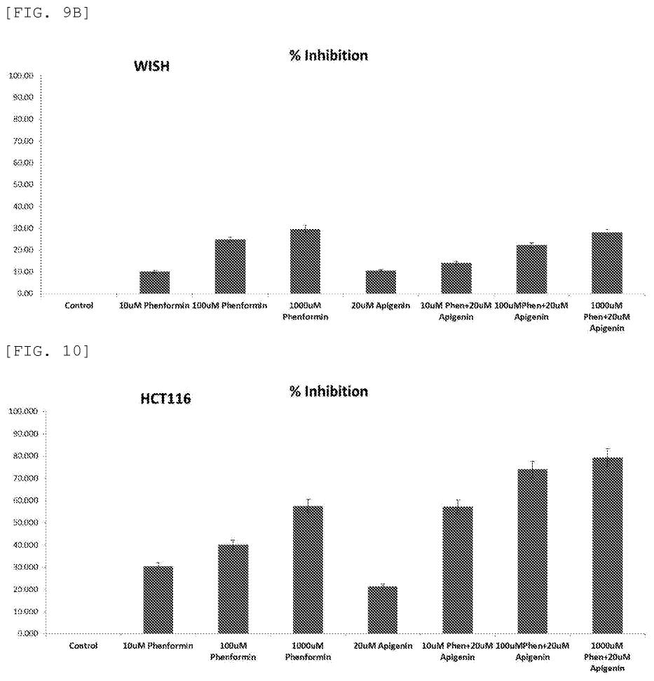

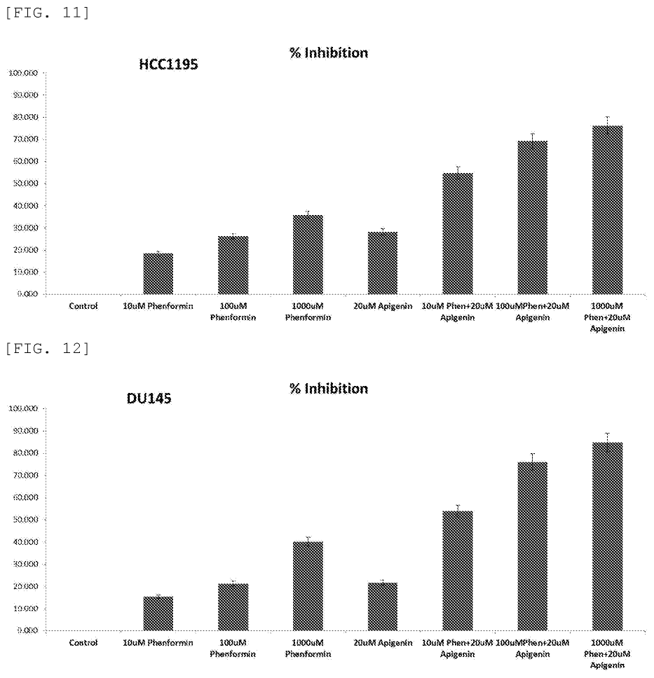

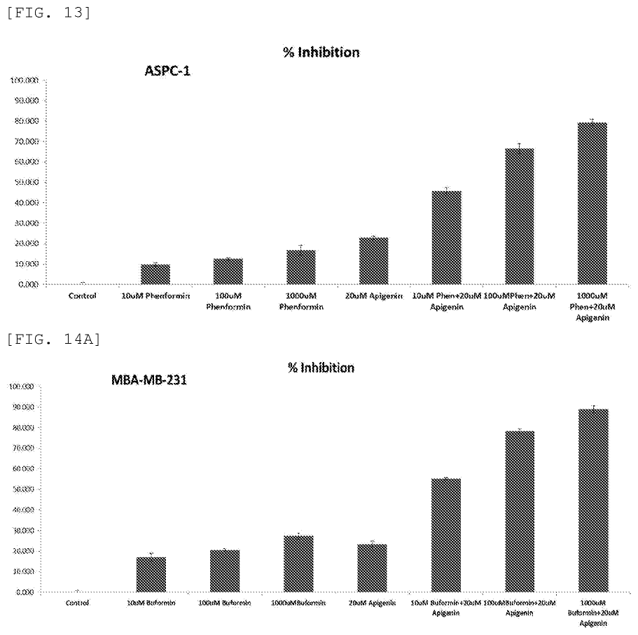

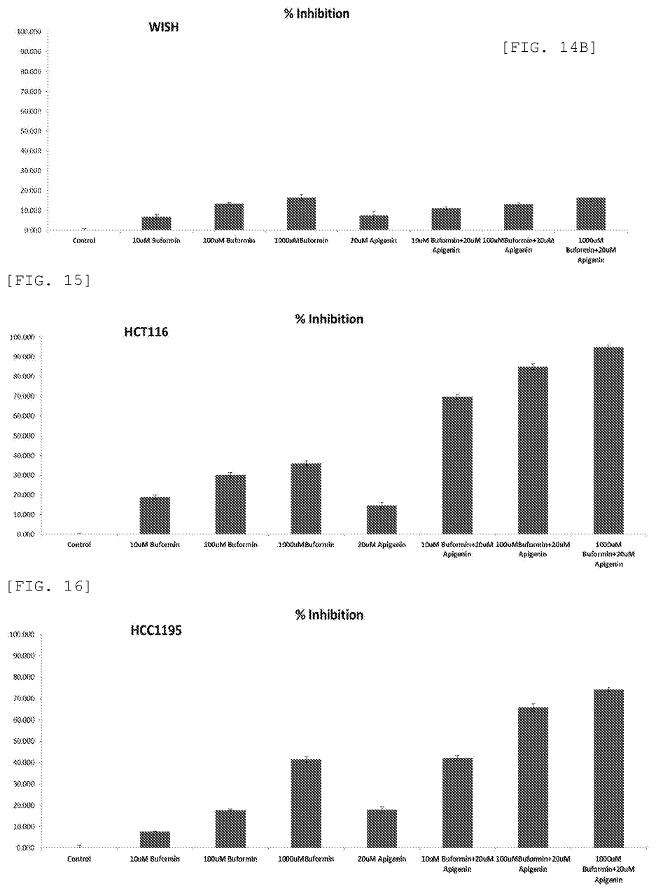

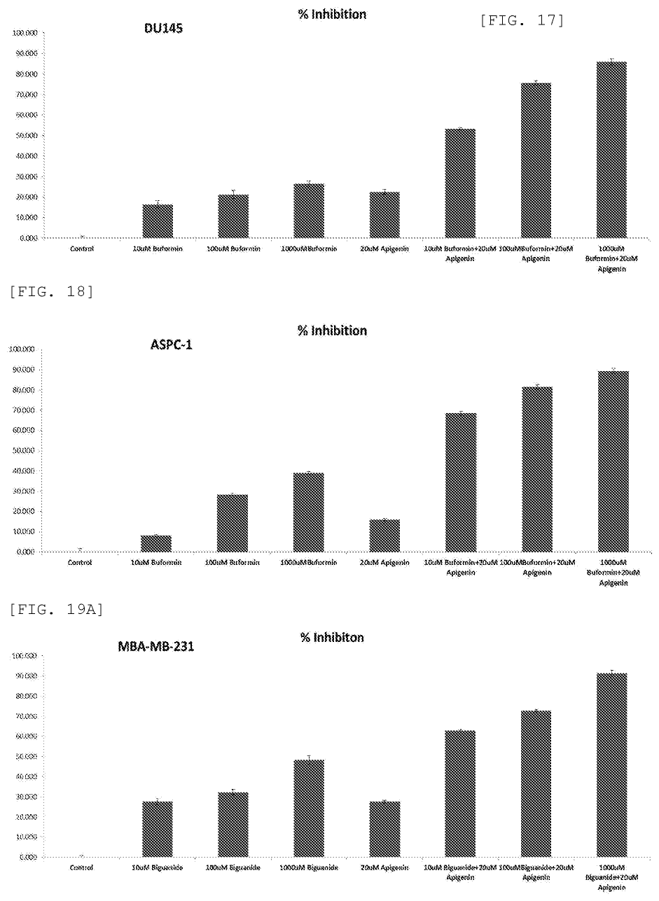

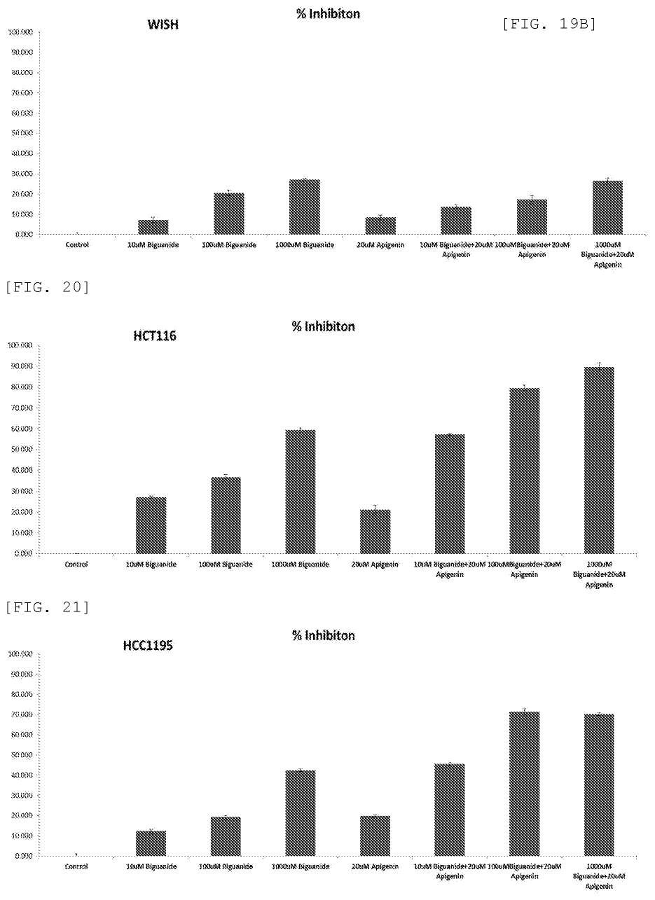

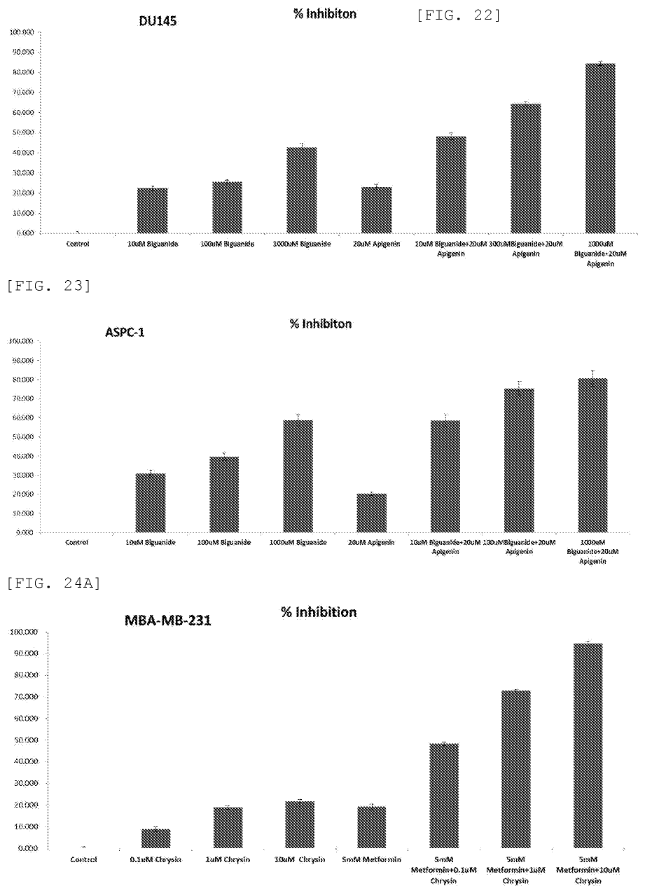

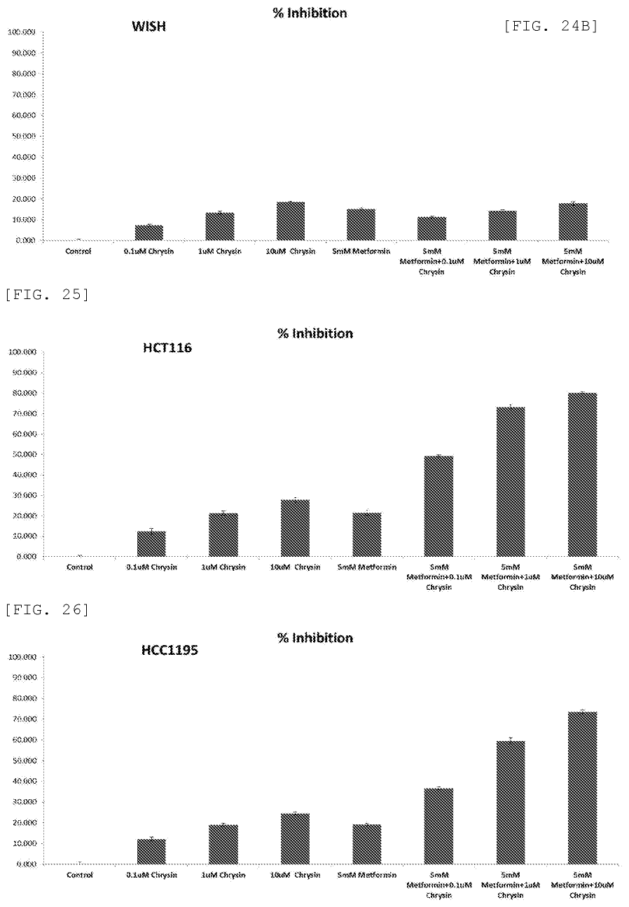

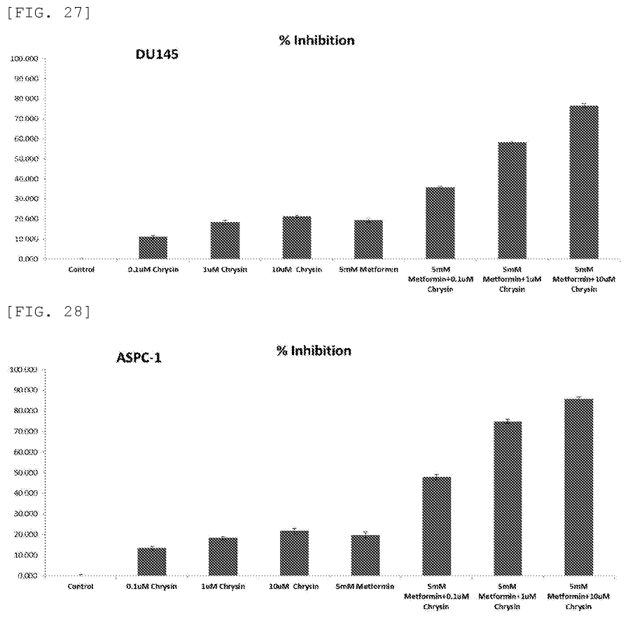

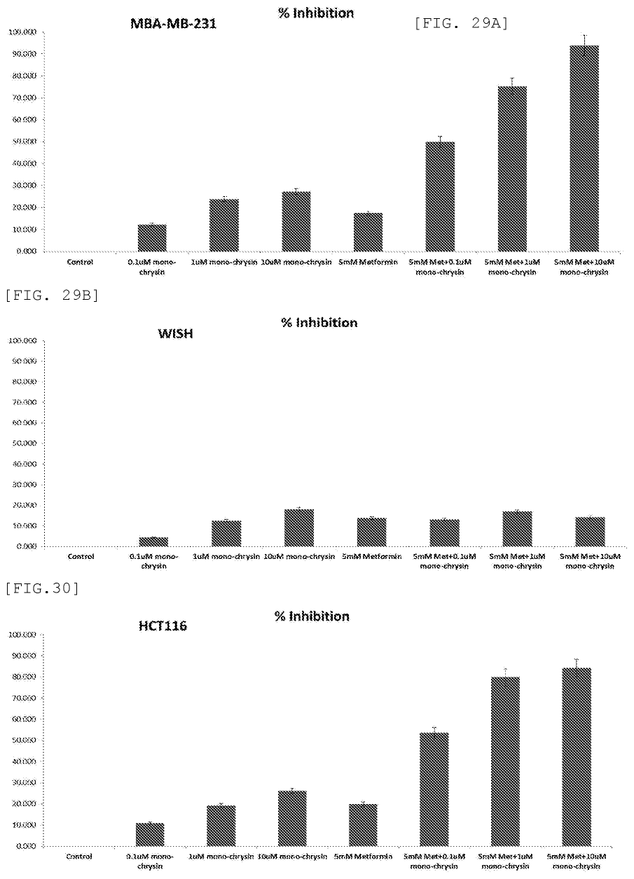

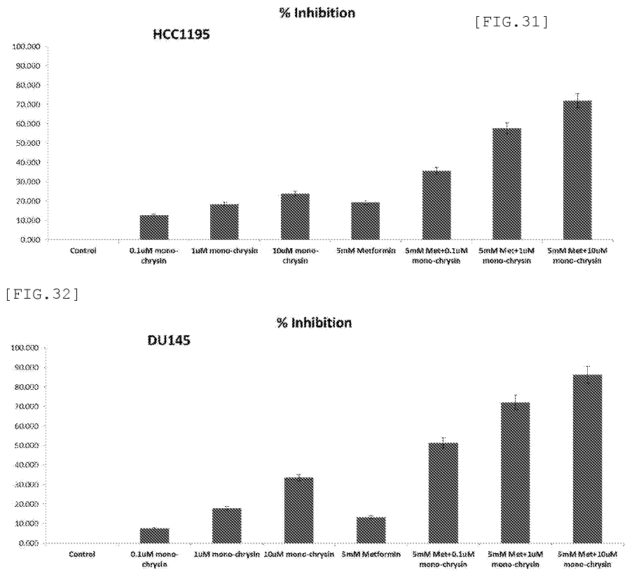

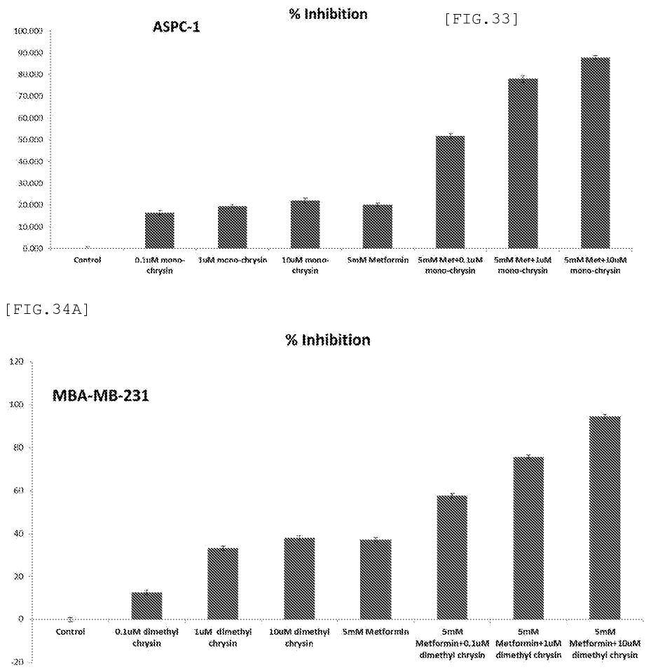

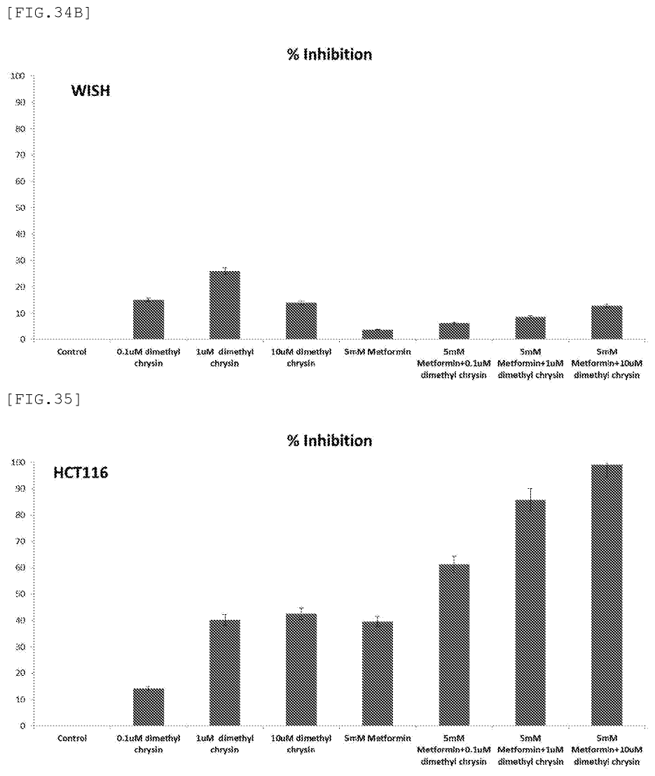

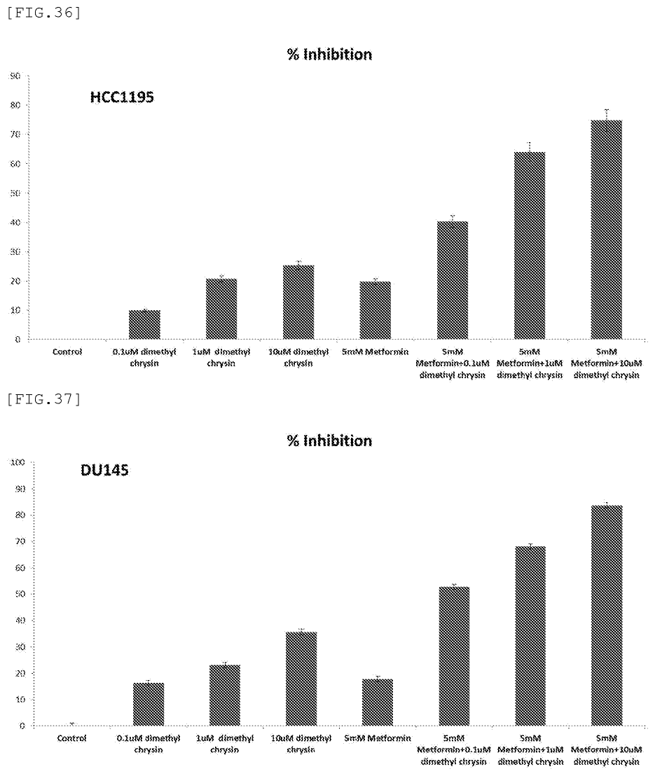

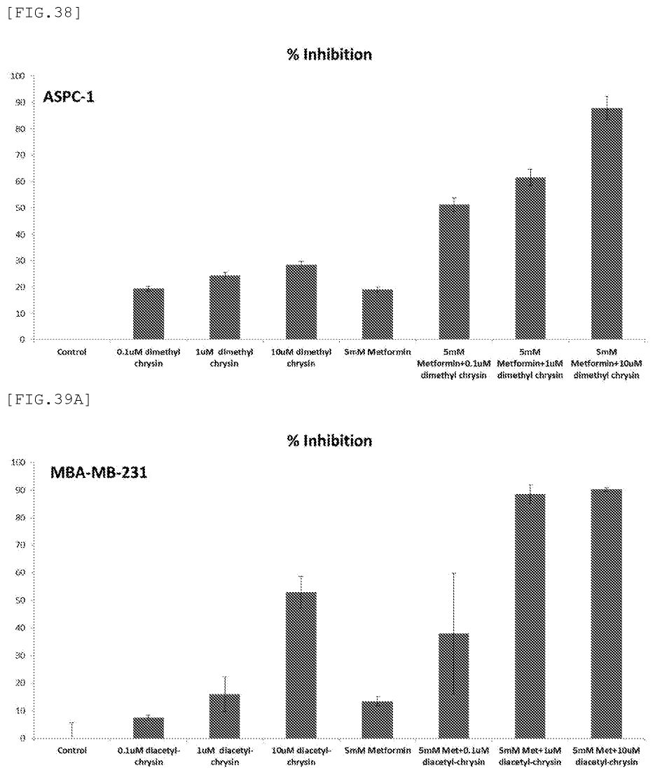

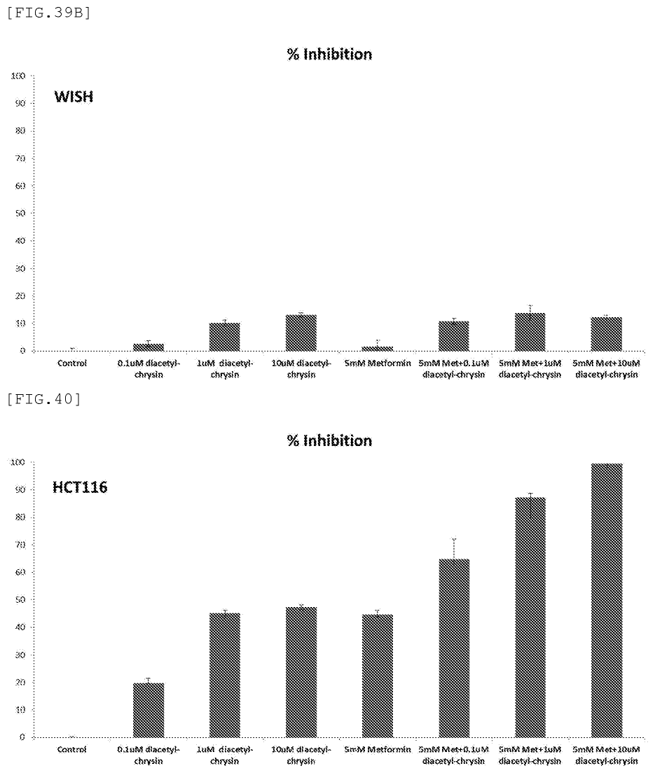

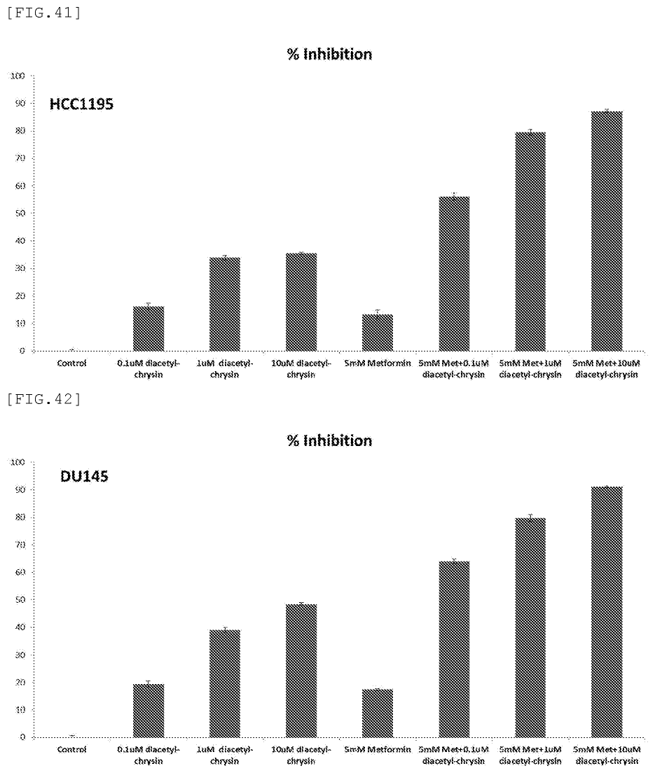

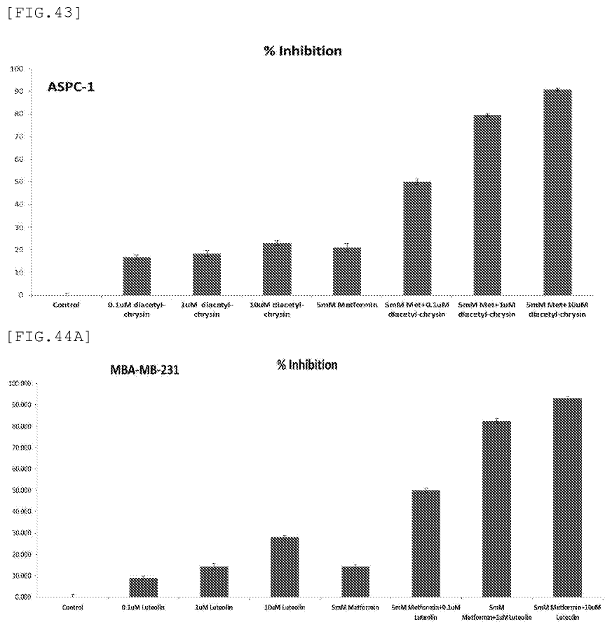

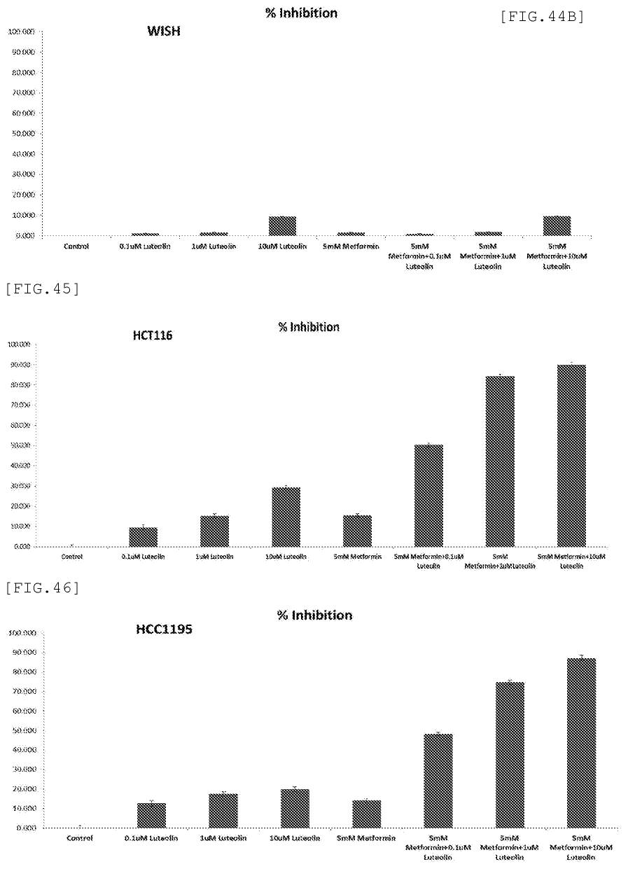

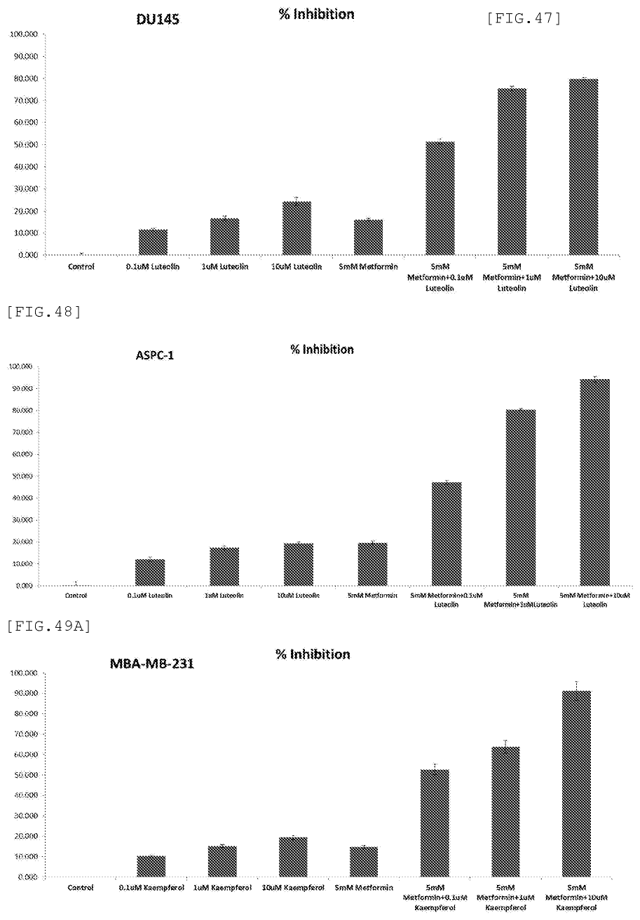

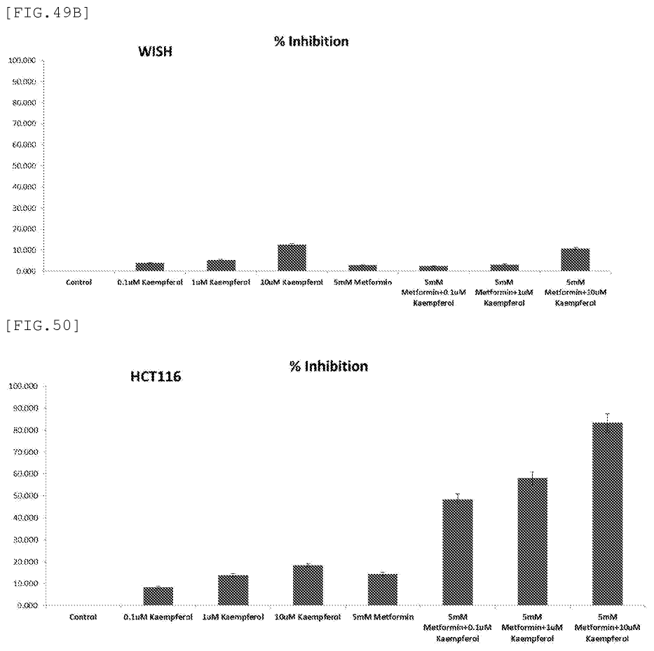

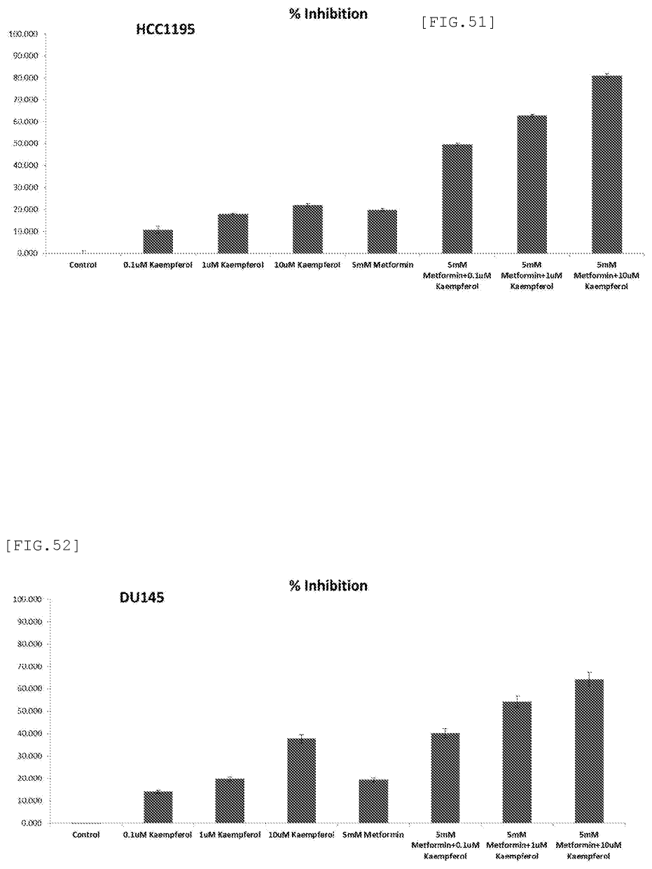

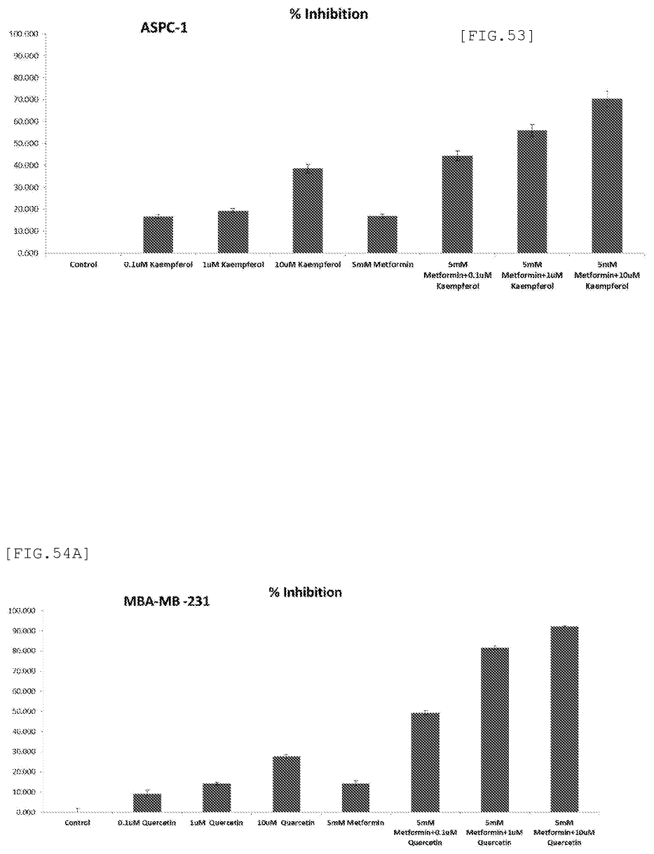

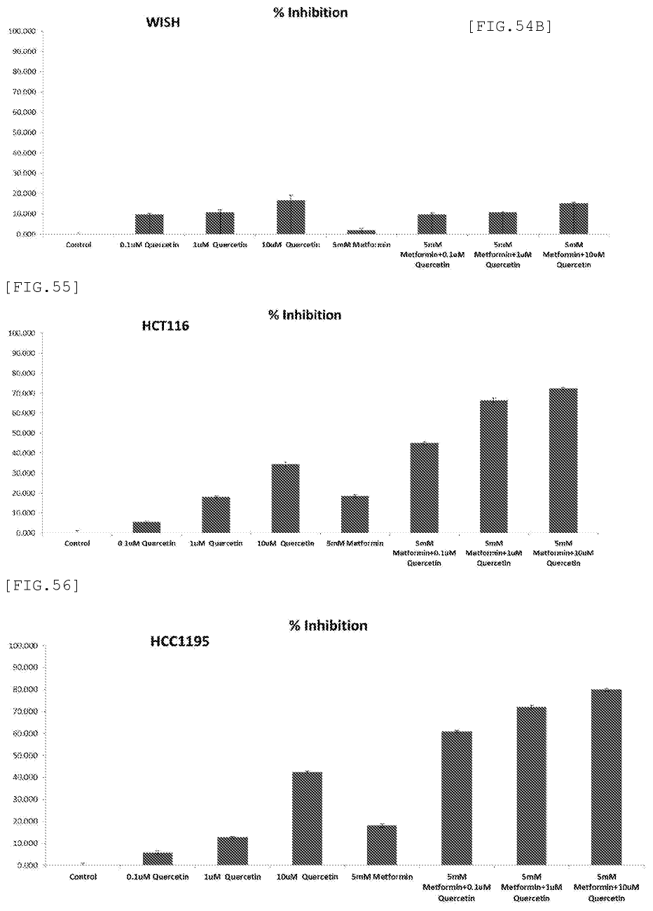

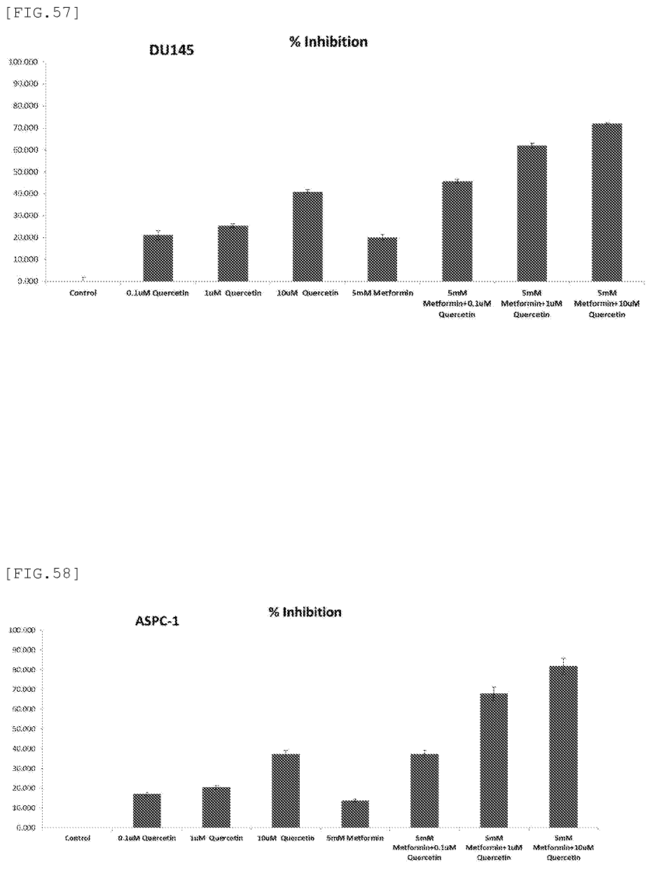

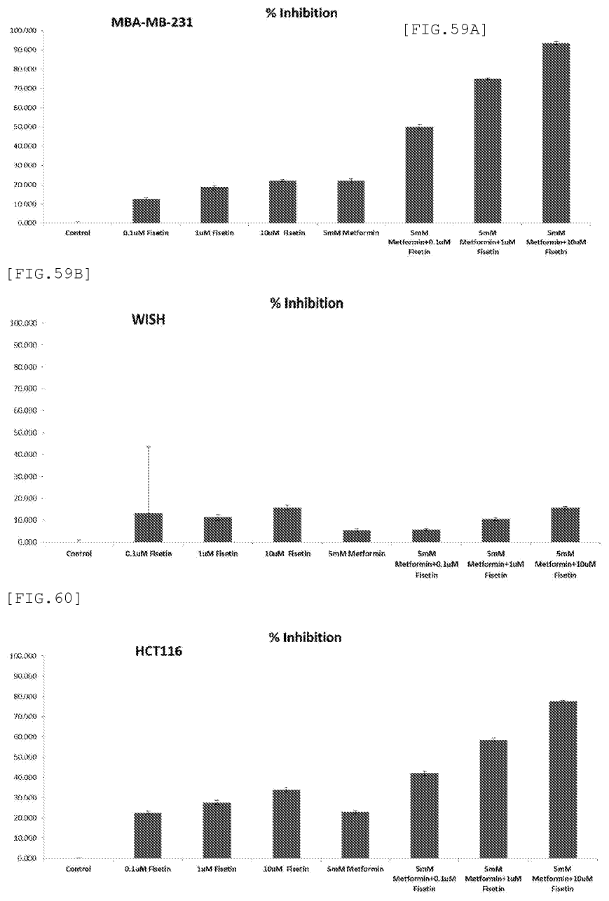

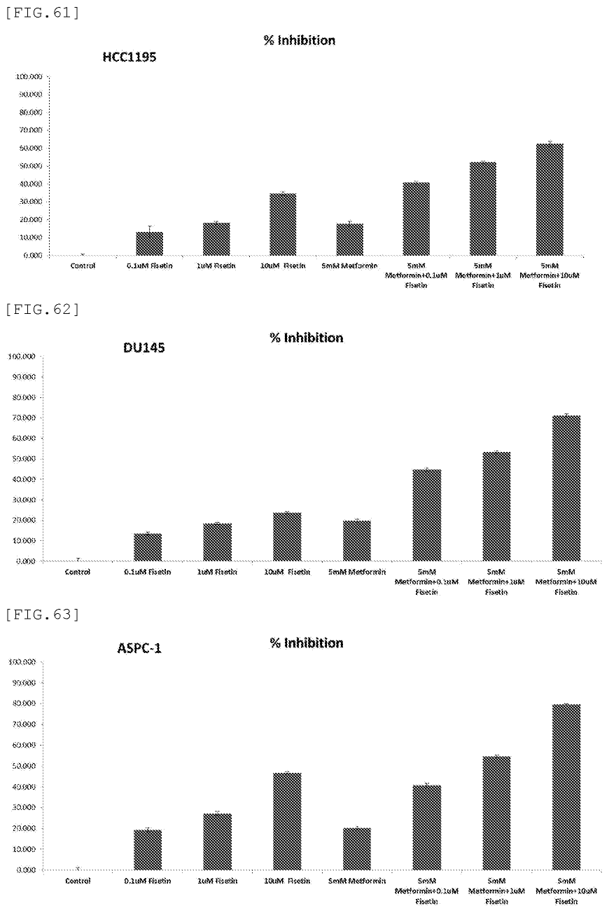

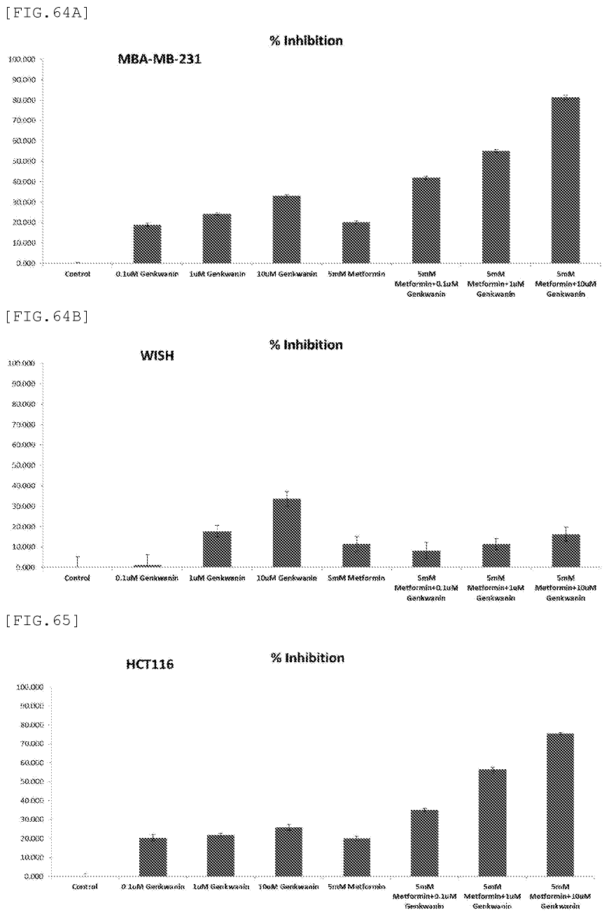

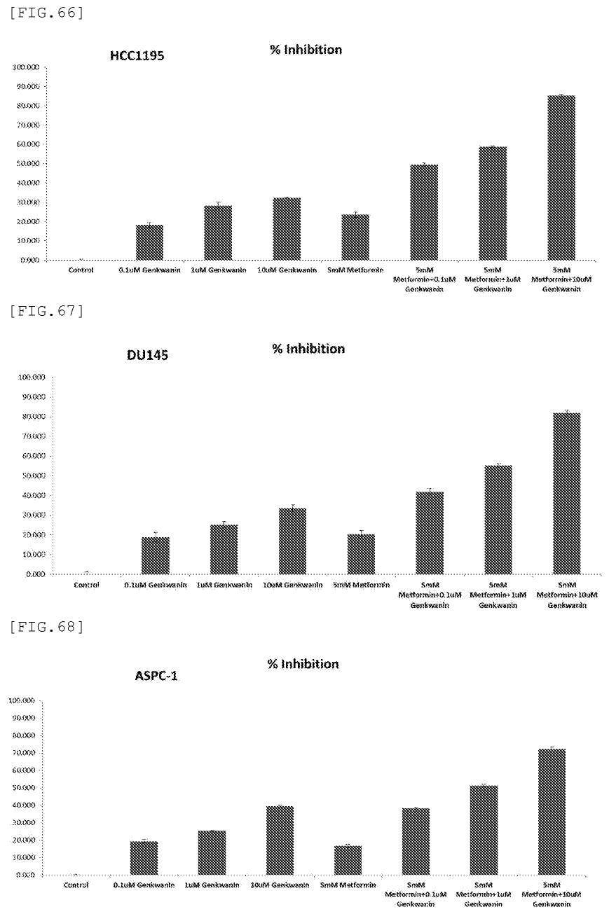

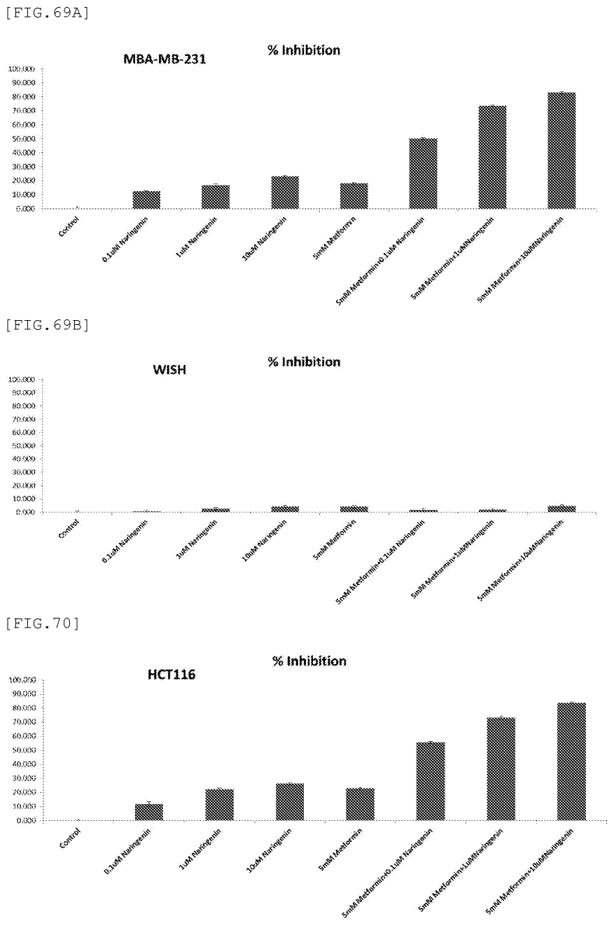

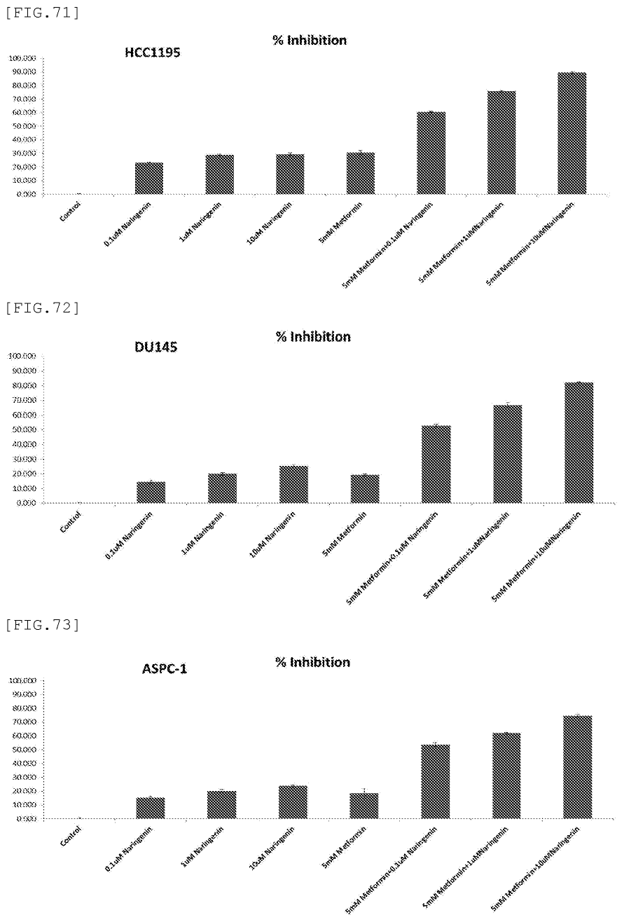

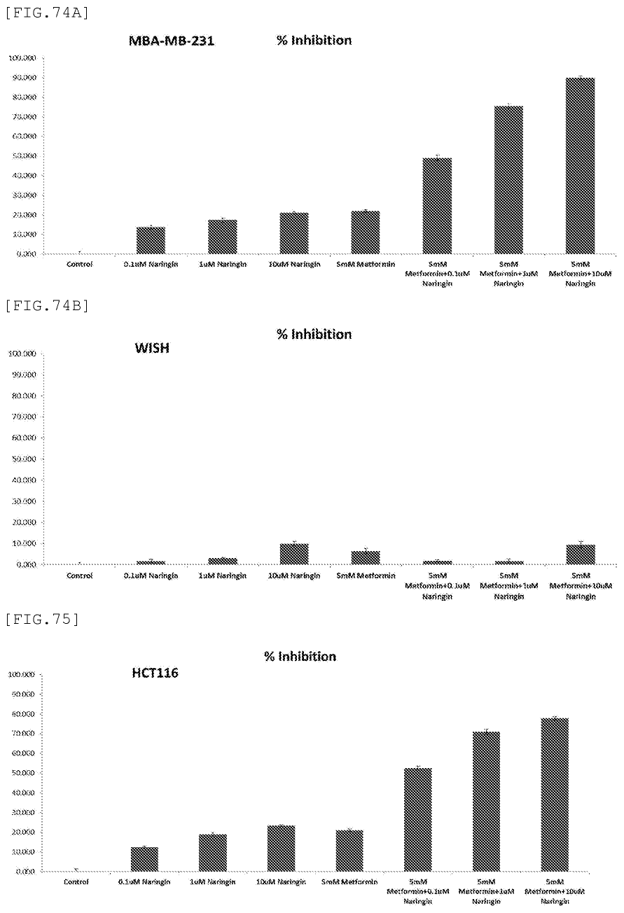

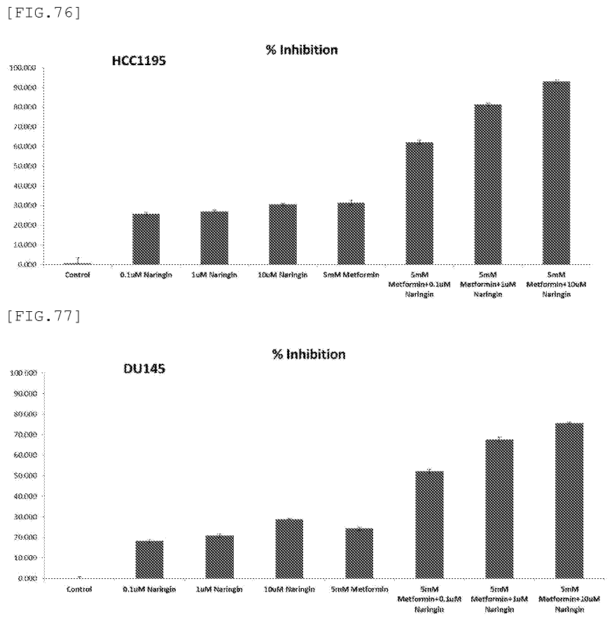

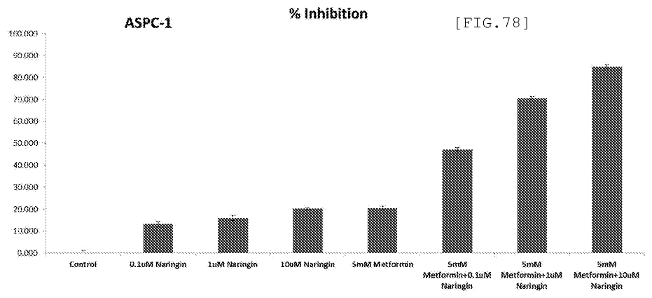

FIG. 1B is a diagram illustrating the degree of growth inhibition (% growth inhibition) when WISH human normal primary epithelial cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cells when treated with 5 mM metformin and 0.01, 0.1, 1, or 10 μM 5,7,4′-trihydroxyflavone in combination for 24 hours;

3: the degree (percentage) of growth inhibition of cells when treated with 5 mM metformin and 0.01, 0.1, 1, or 10 μM 5,7,4′-trihydroxyflavone in combination for 48 hours;

4: the degree (percentage) of growth inhibition of cells when treated with 5 mM metformin and 0.01, 0.1, 1, or 10 μM 5,7,4′-trihydroxyflavone in combination for 72 hours; and 5: the degree (percentage) of growth inhibition of cells when treated with 5 mM metformin and 0.01, 0.1, 1, or 10 μM 5,7,4′-trihydroxyflavone in combination for 96 hours.

FIG. 2 is a diagram illustrating the degree of growth inhibition (% growth inhibition) when HCT 116 colon cancer cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours;

4: the degree (percentage) of growth inhibition of cancer cells when treated with 5 mM metformin and 0.01, 0.1, 1, or 10 μM 5,7,4′-trihydroxyflavone in combination for 72 hours; and

5: the degree (percentage) of growth inhibition of cancer cells when treated with 5 mM metformin and 0.01, 0.1, 1, or 10 μM 5,7,4′-trihydroxyflavone in combination for 96 hours.

FIG. 3 is a diagram illustrating the degree of growth inhibition (% growth inhibition) when Caco-2 colon cancer cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours;

4: the degree (percentage) of growth inhibition of cancer cells when treated with 5 mM metformin and 0.01, 0.1, 1, or 10 μM 5,7,4′-trihydroxyflavone in combination for 72 hours; and

5: the degree (percentage) of growth inhibition of cancer cells when treated with 5 mM metformin and 0.01, 0.1, 1, or 10 μM 5,7,4′-trihydroxyflavone in combination for 96 hours.

FIG. 4 is a diagram illustrating the degree of growth inhibition (% growth inhibition) when HCC1195 lung cancer cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours;

4: the degree (percentage) of growth inhibition of cancer cells when treated with 5 mM metformin and 0.01, 0.1, 1, or 10 μM 5,7,4′-trihydroxyflavone in combination for 72 hours; and

5: the degree (percentage) of growth inhibition of cancer cells when treated with 5 mM metformin and 0.01, 0.1, 1, or 10 μM 5,7,4′-trihydroxyflavone in combination for 96 hours.

FIG. 5 is a diagram illustrating the degree of growth inhibition (% growth inhibition) when DU145 prostate cancer cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours;

4: the degree (percentage) of growth inhibition of cancer cells when treated with 5 mM metformin and 0.01, 0.1, 1, or 10 μM 5,7,4′-trihydroxyflavone in combination for 72 hours; and

5: the degree (percentage) of growth inhibition of cancer cells when treated with 5 mM metformin and 0.01, 0.1, 1, or 10 μM 5,7,4′-trihydroxyflavone in combination for 96 hours.

FIG. 6 is a diagram illustrating the degree of growth inhibition (% growth inhibition) when LNCaP prostate cancer cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours;

4: the degree (percentage) of growth inhibition of cancer cells when treated with 5 mM metformin and 0.01, 0.1, 1, or 10 μM 5,7,4′-trihydroxyflavone in combination for 72 hours; and

5: the degree (percentage) of growth inhibition of cancer cells when treated with 5 mM metformin and 0.01, 0.1, 1, or 10 μM 5,7,4′-trihydroxyflavone in combination for 96 hours.

FIG. 7 is a diagram illustrating the degree of growth inhibition (% growth inhibition) when AsPC-1 pancreatic cancer cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours;

4: the degree (percentage) of growth inhibition of cancer cells when treated with 5 mM metformin and 0.01, 0.1, 1, or 10 μM 5,7,4′-trihydroxyflavone in combination for 72 hours; and

5: the degree (percentage) of growth inhibition of cancer cells when treated with 5 mM metformin and 0.01, 0.1, 1, or 10 μM 5,7,4′-trihydroxyflavone in combination for 96 hours.

FIG. 8 is a diagram illustrating the degree of growth inhibition (% growth inhibition) when MIA Paca-2 pancreatic cancer cells are treated with 5 mM metformin; 0.01, 0.1, 1, or 10 μM 5,7,4′-trihydroxyflavone; or 0.01, 0.1, 1, or 10 μM 5,7,4′-trihydroxyflavone+5 mM metformin for 24, 48, 72 or 96 hours.

1: The degree (percentage) of growth inhibition of cancer cells when treated with 5 mM metformin (control, right blue graph) or 0.01, 0.1, 1, or 10 μM 5,7,4′-trihydroxyflavone (other blue graphs) singly;

2: the degree (percentage) of growth inhibition of cancer cells when treated with 5 mM metformin and 0.01, 0.1, 1, or 10 μM 5,7,4′-trihydroxyflavone in combination for 24 hours;

3: the degree (percentage) of growth inhibition of cancer cells when treated with 5 mM metformin and 0.01, 0.1, 1, or 10 μM 5,7,4′-trihydroxyflavone in combination for 48 hours;

4: the degree (percentage) of growth inhibition of cancer cells when treated with 5 mM metformin and 0.01, 0.1, 1, or 10 μM 5,7,4′-trihydroxyflavone in combination for 72 hours; and

5: the degree (percentage) of growth inhibition of cancer cells when treated with 5 mM metformin and 0.01, 0.1, 1, or 10 μM 5,7,4′-trihydroxyflavone in combination for 96 hours.

FIG. 9A is a diagram illustrating the degree of growth inhibition (% growth inhibition) when MDA-MB-231 breast cancer cells are treated with 10, 100, or 1000 μM phenformin; 20 μM 5,7,4′-trihydroxyflavone; or 10, 100, or 1000 μM phenformin+20 μM 5,7,4′-trihydroxyflavone for 72 hours.

FIG. 9B is a diagram illustrating the degree of growth inhibition (% growth inhibition) when WISH human normal primary epithelial cells are treated with 10, 100, or 1000 μM phenformin; 20 μM 5,7,4′-trihydroxyflavone; or 10, 100, or 1000 μM phenformin+20 μM 5,7,4′-trihydroxyflavone for 72 hours.

FIG. 10 is a diagram illustrating the degree of growth inhibition (% growth inhibition) when HCT 116 colon cancer cells are treated with 10, 100, or 1000 μM phenformin; 20 μM 5,7,4′-trihydroxyflavone; or 10, 100, or 1000 μM phenformin+20 μM 5,7,4′-trihydroxyflavone for 72 hours.

FIG. 11 is a diagram illustrating the degree of growth inhibition (% growth inhibition) when HCC1195 lung cancer cells are treated with 10, 100, or 1000 μM phenformin; 20 μM 5,7,4′-trihydroxyflavone; or 10, 100, or 1000 μM phenformin+20 μM 5,7,4′-trihydroxyflavone for 72 hours.

FIG. 12 is a diagram illustrating the degree of growth inhibition (% growth inhibition) when DU145 prostate cancer cells are treated with 10, 100, or 1000 μM phenformin; 20 μM 5,7,4′-trihydroxyflavone; or 10, 100, or 1000 μM phenformin+20 μM 5,7,4′-trihydroxyflavone for 72 hours.

FIG. 13 is a diagram illustrating the degree of growth inhibition (% growth inhibition) when AsPC-1 pancreatic cancer cells are treated with 10, 100, or 1000 μM phenformin; 20 μM 5,7,4′-trihydroxyflavone; or 10, 100, or 1000 μM phenformin+20 μM 5,7,4′-trihydroxyflavone for 72 hours.

FIG. 14A is a diagram illustrating the degree of growth inhibition (% growth inhibition) when MDA-MB-231 breast cancer cells are treated with 10, 100, or 1000 μM buformin; 20 μM 5,7,4′-trihydroxyflavone; or 10, 100, or 1000 μM buformin+20 μM 5,7,4′-trihydroxyflavone for 72 hours.

FIG. 14B is a diagram illustrating the degree of growth inhibition (% growth inhibition) when WISH human normal primary epithelial cells are treated with 10, 100, or 1000 μM buformin; 20 μM 5,7,4′-trihydroxyflavone; or 10, 100, or 1000 μM buformin+20 μM 5,7,4′-trihydroxyflavone for 72 hours.

FIG. 15 is a diagram illustrating the degree of growth inhibition (% growth inhibition) when HCT 116 colon cancer cells are treated with 10, 100, or 1000 μM buformin; 20 μM 5,7,4′-trihydroxyflavone; or 10, 100, or 1000 μM buformin+20 μM 5,7,4′-trihydroxyflavone for 72 hours.

FIG. 16 is a diagram illustrating the degree of growth inhibition (% growth inhibition) when HCC1195 lung cancer cells are treated with 10, 100, or 1000 μM buformin; 20 μM 5,7,4′-trihydroxyflavone; or 10, 100, or 1000 μM buformin+20 μM 5,7,4′-trihydroxyflavone for 72 hours.

FIG. 17 is a diagram illustrating the degree of growth inhibition (% growth inhibition) when DU145 prostate cancer cells are treated with 10, 100, or 1000 μM buformin; 20 μM 5,7,4′-trihydroxyflavone; or 10, 100, or 1000 μM buformin+20 μM 5,7,4′-trihydroxyflavone for 72 hours.

FIG. 18 is a diagram illustrating the degree of growth inhibition (% growth inhibition) when AsPC-1 pancreatic cancer cells are treated with 10, 100, or 1000 μM buformin; 20 μM 5,7,4′-trihydroxyflavone; or 10, 100, or 1000 μM buformin+20 μM 5,7,4′-trihydroxyflavone for 72 hours.

FIG. 19A is a diagram illustrating the degree of growth inhibition (% growth inhibition) when MDA-MB-231 breast cancer cells are treated with 10, 100, or 1000 μM biguanide; 20 μM 5,7,4′-trihydroxyflavone; or 10, 100, or 1000 μM biguanide+20 μM 5,7,4′-trihydroxyflavone for 72 hours.

FIG. 19B is a diagram illustrating the degree of growth inhibition (% growth inhibition) when WISH human normal primary epithelial cells are treated with 10, 100, or 1000 μM biguanide; 20 μM 5,7,4′-trihydroxyflavone; or 10, 100, or 1000 μM biguanide+20 μM 5,7,4′-trihydroxyflavone for 72 hours.

FIG. 20 is a diagram illustrating the degree of growth inhibition (% growth inhibition) when HCT 116 colon cancer cells are treated with 10, 100, or 1000 μM biguanide; 20 μM 5,7,4′-trihydroxyflavone; or 10, 100, or 1000 μM biguanide+20 μM 5,7,4′-trihydroxyflavone for 72 hours.

FIG. 21 is a diagram illustrating the degree of growth inhibition (% growth inhibition) when HCC1195 lung cancer cells are treated with 10, 100, or 1000 μM biguanide; 20 μM 5,7,4′-trihydroxyflavone; or 10, 100, or 1000 μM biguanide+20 μM 5,7,4′-trihydroxyflavone for 72 hours.

FIG. 22 is a diagram illustrating the degree (%) of growth inhibition when DU145 prostate cancer cells are treated with 10, 100, or 1000 μM biguanide; 20 μM 5,7,4′-trihydroxyflavone; or 10, 100, or 1000 μM biguanide+20 μM 5,7,4′-trihydroxyflavone for 72 hours.

FIG. 23 is a diagram illustrating the degree of growth inhibition (% growth inhibition) when AsPC-1 pancreatic cancer cells are treated with 10, 100, or 1000 μM biguanide; 20 μM 5,7,4′-trihydroxyflavone; or 10, 100, or 1000 μM biguanide+20 μM apigenin for 72 hours.

FIG. 24A is a diagram illustrating the degree of growth inhibition (% growth inhibition) when MDA-MB-231 breast cancer cells are treated with 5 mM metformin; 0.1, 1, or 10 μM 5,7-dihydroxyflavone (chrysin); or 5 mM metformin+0.1, 1, or 10 μM 5,7-dihydroxyflavone for 72 hours.

FIG. 24B is a diagram illustrating the degree of growth inhibition (% growth inhibition) when WISH human normal primary epithelial cells are treated with 5 mM metformin; 0.1, 1, or 10 μM 5,7-dihydroxyflavone; or 5 mM metformin+0.1, 1, or 10 μM 5,7-dihydroxyflavone for 72 hours.

FIG. 25 is a diagram illustrating the degree of growth inhibition (% growth inhibition) when HCT 116 colon cancer cells are treated with 5 mM metformin; 0.1, 1, or 10 μM 5,7-dihydroxyflavone; or 5 mM metformin+0.1, 1, or 10 μM 5,7-dihydroxyflavone for 72 hours.

FIG. 26 is a diagram illustrating the degree of growth inhibition (% growth inhibition) when HCC1195 lung cancer cells are treated with 5 mM metformin; 0.1, 1, or 10 μM 5,7-dihydroxyflavone; or 5 mM metformin+0.1, 1, or 10 μM 5,7-dihydroxyflavone for 72 hours.

FIG. 27 is a diagram illustrating the degree of growth inhibition (% growth inhibition) when DU145 prostate cancer cells are treated with 5 mM metformin; 0.1, 1, or 10 μM 5,7-dihydroxyflavone; or 5 mM metformin+0.1, 1, or 10 μM 5,7-dihydroxyflavone for 72 hours.

FIG. 28 is a diagram illustrating the degree of growth inhibition (% growth inhibition) when AsPC-1 pancreatic cancer cells are treated with 5 mM metformin; 0.1, 1, or 10 μM 5,7-dihydroxyflavone; or 5 mM metformin+0.1, 1, or 10 μM 5,7-dihydroxyflavone for 72 hours.

FIG. 29A is a diagram illustrating the degree of growth inhibition (% growth inhibition) when MDA-MB-231 breast cancer cells are treated with 5 mM metformin; 0.1, 1, or 10 μM 7-O-acetyl chrysin (monoacetyl chrysin); or 5 mM metformin+0.1, 1, or 10 μM 7-O-acetyl chrysin for 72 hours.

FIG. 29B is a diagram illustrating the degree of growth inhibition (% growth inhibition) when WISH human normal primary epithelial cells are treated with 5 mM metformin; 0.1, 1, or 10 μM 7-O-acetyl chrysin; or 5 mM metformin+0.1, 1, or 10 μM 7-O-acetyl chrysin for 72 hours.

FIG. 30 is a diagram illustrating the degree of growth inhibition (% growth inhibition) when HCT 116 colon cancer cells are treated with 5 mM metformin; 0.1, 1, or 10 μM 7-O-acetyl chrysin; or 5 mM metformin+0.1, 1, or 10 μM 7-O-acetyl chrysin for 72 hours.

FIG. 31 is a diagram illustrating the degree of growth inhibition (% growth inhibition) when HCC1195 lung cancer cells are treated with 5 mM metformin; 0.1, 1, or 10 μM 7-O-acetyl chrysin; or 5 mM metformin+0.1, 1, or 10 μM 7-O-acetyl chrysin for 72 hours.

FIG. 32 is a diagram illustrating the degree of growth inhibition (% growth inhibition) when DU145 prostate cancer cells are treated with 5 mM metformin; 0.1, 1, or 10 μM 7-O-acetyl chrysin; or 5 mM metformin+0.1, 1, or 10 μM 7-O-acetyl chrysin for 72 hours.

FIG. 33 is a diagram illustrating the degree of growth inhibition (% growth inhibition) when AsPC-1 pancreatic cancer cells are treated with 5 mM metformin; 0.1, 1, or 10 μM 7-O-acetyl chrysin; or 5 mM metformin+0.1, 1, or 10 μM 7-O-acetyl chrysin for 72 hours.

FIG. 34A is a diagram illustrating the degree of growth inhibition (% growth inhibition) when MDA-MB-231 breast cancer cells are treated with 5 mM metformin; 0.1, 1, or 10 μM 5,7-di-O-methoxy chrysin (dimethyl chrysin); or 5 mM metformin+0.1, 1, or 10 μM 5,7-di-O-methoxy chrysin for 72 hours.

FIG. 34B is a diagram illustrating the degree of growth inhibition (% growth inhibition) when WISH human normal primary epithelial cells are treated with 5 mM metformin; 0.1, 1, or 10 μM 5,7-di-O-methoxy chrysin; or 5 mM metformin+0.1, 1, or 10 μM 5,7-di-O-methoxy chrysin for 72 hours.

FIG. 35 is a diagram illustrating the degree of growth inhibition (% growth inhibition) when HCT 116 colon cancer cells are treated with 5 mM metformin; 0.1, 1, or 10 μM 5,7-di-O-methoxy chrysin; or 5 mM metformin+0.1, 1, or 10 μM 5,7-di-O-methoxy chrysin for 72 hours.

FIG. 36 is a diagram illustrating the degree of growth inhibition (% growth inhibition) when HCC1195 lung cancer cells are treated with 5 mM metformin; 0.1, 1, or 10 μM 5,7-di-O-methoxy chrysin; or 5 mM metformin+0.1, 1, or 10 μM 5,7-di-O-methoxy chrysin for 72 hours.

FIG. 37 is a diagram illustrating the degree of growth inhibition (% growth inhibition) when DU145 prostate cancer cells are treated with 5 mM metformin; 0.1, 1, or 10 μM 5,7-di-O-methoxy chrysin; or 5 mM metformin+0.1, 1, or 10 μM 5,7-di-O-methoxy chrysin for 72 hours.

FIG. 38 is a diagram illustrating the degree of growth inhibition (% growth inhibition) when AsPC-1 pancreatic cancer cells are treated with 5 mM metformin; 0.1, 1, or 10 μM 5,7-di-O-methoxy chrysin; or 5 mM metformin+0.1, 1, or 10 μM 5,7-di-O-methoxy chrysin for 72 hours.

FIG. 39A is a diagram illustrating the degree of growth inhibition (% growth inhibition) when MDA-MB-231 breast cancer cells are treated with 5 mM metformin; 0.1, 1, or 10 μM 5,7-di-O-acetyl chrysin (diacetyl chrysin); or 5 mM metformin+0.1, 1, or 10 μM 5,7-di-O-acetyl chrysin for 72 hours.

FIG. 39B is a diagram illustrating the degree of growth inhibition (% growth inhibition) when WISH human normal primary epithelial cells are treated with 5 mM metformin; 0.1, 1, or 10 μM 5,7-di-O-acetyl chrysin; or 5 mM metformin+0.1, 1, or 10 μM 5,7-di-O-acetyl chrysin for 72 hours.

FIG. 40 is a diagram illustrating the degree of growth inhibition (% growth inhibition) when HCT 116 colon cancer cells are treated with 5 mM metformin; 0.1, 1, or 10 μM 5,7-di-O-acetyl chrysin; or 5 mM metformin+0.1, 1, or 10 μM 5,7-di-O-acetyl chrysin for 72 hours.

FIG. 41 is a diagram illustrating the degree of growth inhibition (% growth inhibition) when HCC1195 lung cancer cells are treated with 5 mM metformin; 0.1, 1, or 10 μM 5,7-di-O-acetyl chrysin; or 5 mM metformin+0.1, 1, or 10 μM 5,7-di-O-acetyl chrysin for 72 hours.

FIG. 42 is a diagram illustrating the degree of growth inhibition (% growth inhibition) when DU145 prostate cancer cells are treated with 5 mM metformin; 0.1, 1, or 10 μM 5,7-di-O-acetyl chrysin; or 5 mM metformin+0.1, 1, or 10 μM 5,7-di-O-acetyl chrysin for 72 hours.

FIG. 43 is a diagram illustrating the degree of growth inhibition (% growth inhibition) when AsPC-1 pancreatic cancer cells are treated with 5 mM metformin; 0.1, 1, or 10 μM 5,7-di-O-acetyl chrysin; or 5 mM metformin+0.1, 1, or 10 μM 5,7-di-O-acetyl chrysin for 72 hours.

FIG. 44A is a diagram illustrating the degree of growth inhibition (% growth inhibition) when MDA-MB-231 breast cancer cells are treated with 5 mM metformin; 0.1, 1, or 10 μM 3′,4′,5,7-tetrahydroxyflavone (luteolin); or 5 mM metformin+0.1, 1, or 10 μM 3′,4′,5,7-tetrahydroxyflavone for 72 hours.

FIG. 44B is a diagram illustrating the degree of growth inhibition (% growth inhibition) when WISH human normal primary epithelial cells are treated with 5 mM metformin; 0.1, 1, or 10 μM 3′,4′,5,7-tetrahydroxyflavone; or 5 mM metformin+0.1, 1, or 10 μM 3′,4′,5,7-tetrahydroxyflavone for 72 hours.

FIG. 45 is a diagram illustrating the degree of growth inhibition (% growth inhibition) when HCT 116 colon cancer cells are treated with 5 mM metformin; 0.1, 1, or 10 μM 3′,4′,5,7-tetrahydroxyflavone; or 5 mM metformin+0.1, 1, or 10 μM 3′,4′,5,7-tetrahydroxyflavone for 72 hours.

FIG. 46 is a diagram illustrating the degree of growth inhibition (% growth inhibition) when HCC1195 lung cancer cells are treated with 5 mM metformin; 0.1, 1, or 10 μM 3′,4′,5,7-tetrahydroxyflavone; or 5 mM metformin+0.1, 1, or 10 μM 3′,4′,5,7-tetrahydroxyflavone for 72 hours.

FIG. 47 is a diagram illustrating the degree of growth inhibition (% growth inhibition) when DU145 prostate cancer cells are treated with 5 mM metformin; 0.1, 1, or 10 μM 3′,4′,5,7-tetrahydroxyflavone; or 5 mM metformin+0.1, 1, or 10 μM 3′,4′,5,7-tetrahydroxyflavone for 72 hours.

FIG. 48 is a diagram illustrating the degree of growth inhibition (% growth inhibition) when AsPC-1 pancreatic cancer cells are treated with 5 mM metformin; 0.1, 1, or 10 μM 3′,4′,5,7-tetrahydroxyflavone; or 5 mM metformin+0.1, 1, or 10 μM 3′,4′,5,7-tetrahydroxyflavone for 72 hours.

FIG. 49A is a diagram illustrating the degree of growth inhibition (% growth inhibition) when MDA-MB-231 breast cancer cells are treated with 5 mM metformin; 0.1, 1, or 10 μM 3,4′,5,7-tetrahydroxyflavone (kaempferol)); or 5 mM metformin+0.1, 1, or 10 μM 3,4′,5,7-tetrahydroxyflavone for 72 hours.

FIG. 49B is a diagram illustrating the degree of growth inhibition (% growth inhibition) when WISH human normal primary epithelial cells are treated with 5 mM metformin; 0.1, 1, or 10 μM 3,4′,5,7-tetrahydroxyflavone; or 5 mM metformin+0.1, 1, or 10 μM 3,4′,5,7-tetrahydroxyflavone for 72 hours.

FIG. 50 is a diagram illustrating the degree of growth inhibition (% growth inhibition) when HCT 116 colon cancer cells are treated with 5 mM metformin; 0.1, 1, or 10 μM 3,4′,5,7-tetrahydroxyflavone; or 5 mM metformin+0.1, 1, or 10 μM 3,4′,5,7-tetrahydroxyflavone for 72 hours.

FIG. 51 is a diagram illustrating the degree of growth inhibition (% growth inhibition) when HCC1195 lung cancer cells are treated with 5 mM metformin; 0.1, 1, or 10 μM 3,4′,5,7-tetrahydroxyflavone; or 5 mM metformin+0.1, 1, or 10 μM 3,4′,5,7-tetrahydroxyflavone for 72 hours.

FIG. 52 is a diagram illustrating the degree (%) of growth inhibition when DU145 prostate cancer cells are treated with 5 mM metformin; 0.1, 1, or 10 μM 3,4′,5,7-tetrahydroxyflavone; or 5 mM metformin+0.1, 1, or 10 μM 3,4′,5,7-tetrahydroxyflavone for 72 hours.

FIG. 53 is a diagram illustrating the degree of growth inhibition (% growth inhibition) when AsPC-1 pancreatic cancer cells are treated with 5 mM metformin; 0.1, 1, or 10 μM 3,4′,5,7-tetrahydroxyflavone; or 5 mM metformin+0.1, 1, or 10 μM 3,4′,5,7-tetrahydroxyflavone for 72 hours.

FIG. 54A is a diagram illustrating the degree of growth inhibition (% growth inhibition) when MDA-MB-231 breast cancer cells are treated with 5 mM metformin; 0.1, 1, or 10 μM 5,7,3′,4′-flavon-3-ol (quercetin); or 5 mM metformin+0.1, 1, or 10 μM 5,7,3′,4′-flavon-3-ol for 72 hours.

FIG. 54B is a diagram illustrating the degree of growth inhibition (% growth inhibition) when WISH human normal primary epithelial cells are treated with 5 mM metformin; 0.1, 1, or 10 μM 5,7,3′,4′-flavon-3-ol; or 5 mM metformin+0.1, 1, or 10 μM 5,7,3′,4′-flavon-3-ol for 72 hours.

FIG. 55 is a diagram illustrating the degree of growth inhibition (% growth inhibition) when HCT 116 colon cancer cells are treated with 5 mM metformin; 0.1, 1, or 10 μM 5,7,3′,4′-flavon-3-ol; or 5 mM metformin+0.1, 1, or 10 μM 5,7,3′,4′-flavon-3-ol for 72 hours.

FIG. 56 is a diagram illustrating the degree of growth inhibition (% growth inhibition) when HCC1195 lung cancer cells are treated with 5 mM metformin; 0.1, 1, or 10 μM 5,7,3′,4′-flavon-3-ol; or 5 mM metformin+0.1, 1, or 10 μM 5,7,3′,4′-flavon-3-ol for 72 hours.

FIG. 57 is a diagram illustrating the degree of growth inhibition (% growth inhibition) when DU145 prostate cancer cells are treated with 5 mM metformin; 0.1, 1, or 10 μM 5,7,3′,4′-flavon-3-ol; or 5 mM metformin+0.1, 1, or 10 μM 5,7,3′,4′-flavon-3-ol for 72 hours.

FIG. 58 is a diagram illustrating the degree of growth inhibition (% growth inhibition) when AsPC-1 pancreatic cancer cells are treated with 5 mM metformin; 0.1, 1, or 10 μM 5,7,3′,4′-flavon-3-ol; or 5 mM metformin+0.1, 1, or 10 μM 5,7,3′,4′-flavon-3-ol for 72 hours.

FIG. 59A is a diagram illustrating the degree of growth inhibition (% growth inhibition) when MDA-MB-231 breast cancer cells are treated with 5 mM metformin; 0.1, 1, or 10 μM 7,3′,4′-flavon-3-ol (fisetin); or 5 mM metformin+0.1, 1, or 10 μM 7,3′,4′-flavon-3-ol for 72 hours.

FIG. 59B is a diagram illustrating the degree of growth inhibition (% growth inhibition) when WISH human normal primary epithelial cells are treated with 5 mM metformin; 0.1, 1, or 10 μM 7,3′,4′-flavon-3-ol; or 5 mM metformin+0.1, 1, or 10 μM 7,3′,4′-flavon-3-ol for 72 hours.

FIG. 60 is a diagram illustrating the degree of growth inhibition (% growth inhibition) when HCT 116 colon cancer cells are treated with 5 mM metformin; 0.1, 1, or 10 μM 7,3′,4′-flavon-3-ol; or 5 mM metformin+0.1, 1, or 10 μM 7,3′,4′-flavon-3-ol for 72 hours.

FIG. 61 is a diagram illustrating the degree of growth inhibition (% growth inhibition) when HCC1195 lung cancer cells are treated with 5 mM metformin; 0.1, 1, or 10 μM 7,3′,4′-flavon-3-ol; or 5 mM metformin+0.1, 1, or 10 μM 7,3′,4′-flavon-3-ol for 72 hours.

FIG. 62 is a diagram illustrating the degree of growth inhibition (% growth inhibition) when DU145 prostate cancer cells are treated with 5 mM metformin; 0.1, 1, or 10 μM 7,3′,4′-flavon-3-ol; or 5 mM metformin+0.1, 1, or 10 μM 7,3′,4′-flavon-3-ol for 72 hours.

FIG. 63 is a diagram illustrating the degree of growth inhibition (% growth inhibition) when AsPC-1 pancreatic cancer cells are treated with 5 mM metformin; 0.1, 1, or 10 μM 7,3′,4′-flavon-3-ol; or 5 mM metformin+0.1, 1, or 10 μM 7,3′,4′-flavon-3-ol for 72 hours.

FIG. 64A is a diagram illustrating the degree of growth inhibition (% growth inhibition) when MDA-MB-231 breast cancer cells are treated with 5 mM metformin; 0.1, 1, or 10 μM 4′,5-dihydroxy-7-methoxyflavone (genkwanin); or 5 mM metformin+0.1, 1, or 10 μM 4′,5-dihydroxy-7-methoxyflavone for 72 hours.

FIG. 64B is a diagram illustrating the degree of growth inhibition (% growth inhibition) when WISH human normal primary epithelial cells are treated with 5 mM metformin; 0.1, 1, or 10 μM 4′,5-dihydroxy-7-methoxyflavone; or 5 mM metformin+0.1, 1, or 10 μM 4′,5-dihydroxy-7-methoxyflavone for 72 hours.

FIG. 65 is a diagram illustrating the degree of growth inhibition (% growth inhibition) when HCT 116 colon cancer cells are treated with 5 mM metformin; 0.1, 1, or 10 μM 4′,5-dihydroxy-7-methoxyflavone; or 5 mM metformin+0.1, 1, or 10 μM 4′,5-dihydroxy-7-methoxyflavone for 72 hours.

FIG. 66 is a diagram illustrating the degree of growth inhibition (% growth inhibition) when HCC1195 lung cancer cells are treated with 5 mM metformin; 0.1, 1, or 10 μM 4′,5-dihydroxy-7-methoxyflavone; or 5 mM metformin+0.1, 1, or 10 μM 4′,5-dihydroxy-7-methoxyflavone for 72 hours.

FIG. 67 is a diagram illustrating the degree of growth inhibition (% growth inhibition) when DU145 prostate cancer cells are treated with 5 mM metformin; 0.1, 1, or 10 μM 4′,5-dihydroxy-7-methoxyflavone; or 5 mM metformin+0.1, 1, or 10 μM 4′,5-dihydroxy-7-methoxyflavone for 72 hours.

FIG. 68 is a diagram illustrating the degree of growth inhibition (% growth inhibition) when AsPC-1 pancreatic cancer cells are treated with 5 mM metformin; 0.1, 1, or 10 μM 4′,5-dihydroxy-7-methoxyflavone; or 5 mM metformin+0.1, 1, or 10 μM 4′,5-dihydroxy-7-methoxyflavone for 72 hours.

FIG. 69A is a diagram illustrating the degree of growth inhibition (% growth inhibition) when MDA-MB-231 breast cancer cells are treated with 5 mM metformin; 0.1, 1, or 10 μM 4′,5,7-trihydroxyflavanone (naringenin); or 5 mM metformin+0.1, 1, or 10 μM 4′,5,7-trihydroxyflavanone for 72 hours.

FIG. 69B is a diagram illustrating the degree of growth inhibition (% growth inhibition) when WISH human normal primary epithelial cells are treated with 5 mM metformin; 0.1, 1, or 10 μM 4′,5,7-trihydroxyflavanone; or 5 mM metformin+0.1, 1, or 10 μM 4′,5,7-trihydroxyflavanone for 72 hours.

FIG. 70 is a diagram illustrating the degree of growth inhibition (% growth inhibition) when HCT 116 colon cancer cells are treated with 5 mM metformin; 0.1, 1, or 10 μM 4′,5,7-trihydroxyflavanone; or 5 mM metformin+0.1, 1, or 10 μM 4′,5,7-trihydroxyflavanone for 72 hours.

FIG. 71 is a diagram illustrating the degree of growth inhibition (% growth inhibition) when HCC1195 lung cancer cells are treated with 5 mM metformin; 0.1, 1, or 10 μM 4′,5,7-trihydroxyflavanone; or 5 mM metformin+0.1, 1, or 10 μM 4′,5,7-trihydroxyflavanone for 72 hours.

FIG. 72 is a diagram illustrating the degree of growth inhibition (% growth inhibition) when DU145 prostate cancer cells are treated with 5 mM metformin; 0.1, 1, or 10 μM 4′,5,7-trihydroxyflavanone; or 5 mM metformin+0.1, 1, or 10 μM 4′,5,7-trihydroxyflavanone for 72 hours.

FIG. 73 is a diagram illustrating the degree of growth inhibition (% growth inhibition) when AsPC-1 pancreatic cancer cells are treated with 5 mM metformin; 0.1, 1, or 10 μM 4′,5,7-trihydroxyflavanone; or 5 mM metformin+0.1, 1, or 10 μM 4′,5,7-trihydroxyflavanone for 72 hours.

FIG. 74A is a diagram illustrating the degree of growth inhibition (% growth inhibition) when MDA-MB-231 breast cancer cells are treated with 5 mM metformin; 0.1, 1, or 10 μM 4′,5,7-trihydroxyflavanone-7-rhamnoglucoside (naringin); or 5 mM metformin+0.1, 1, or 10 μM 4′,5,7-trihydroxyflavanone-7-rhamnoglucoside for 72 hours.

FIG. 74B is a diagram illustrating the degree of growth inhibition (% growth inhibition) when WISH human normal primary epithelial cells are treated with 5 mM metformin; 0.1, 1, or 10 μM 4′,5,7-trihydroxyflavanone-7-rhamnoglucoside; or 5 mM metformin+0.1, 1, or 10 μM 4′,5,7-trihydroxyflavanone-7-rhamnoglucoside for 72 hours.

FIG. 75 is a diagram illustrating the degree of growth inhibition (% growth inhibition) when HCT 116 colon cancer cells are treated with 5 mM metformin; 0.1, 1, or 10 μM 4′,5,7-trihydroxyflavanone-7-rhamnoglucoside; or 5 mM metformin+0.1, 1, or 10 μM 4′,5,7-trihydroxyflavanone-7-rhamnoglucoside for 72 hours.

FIG. 76 is a diagram illustrating the degree of growth inhibition (% growth inhibition) when HCC1195 lung cancer cells are treated with 5 mM metformin; 0.1, 1, or 10 μM 4′,5,7-trihydroxyflavanone-7-rhamnoglucoside; or 5 mM metformin+0.1, 1, or 10 μM 4′,5,7-trihydroxyflavanone-7-rhamnoglucoside for 72 hours.

FIG. 77 is a diagram illustrating the degree of growth inhibition (% growth inhibition) when DU145 prostate cancer cells are treated with 5 mM metformin; 0.1, 1, or 10 μM 4′,5,7-trihydroxyflavanone-7-rhamnoglucoside; or 5 mM metformin+0.1, 1, or 10 μM 4′,5,7-trihydroxyflavanone-7-rhamnoglucoside for 72 hours.

FIG. 78 is a diagram illustrating the degree of growth inhibition (% growth inhibition) when AsPC-1 pancreatic cancer cells are treated with 5 mM metformin; 0.1, 1, or 10 μM 4′,5,7-trihydroxyflavanone-7-rhamnoglucoside; or 5 mM metformin+0.1, 1, or 10 μM 4′,5,7-trihydroxyflavanone-7-rhamnoglucoside for 72 hours.

### DESCRIPTION OF EMBODIMENTS

Hereinafter, the present invention will be described in more detail.

### EMBODIMENTS

The present invention relates to a pharmaceutical composition for prevention or treatment of cancer as a complex, mixed or combined preparation for use in the prevention or treatment of cancer, the pharmaceutical composition containing a first component including a biguanide-based compound or a pharmaceutically acceptable salt thereof and a second component including a flavone, a hydroxyflavone, a flavanone, a flavone derivative, a hydroxyflavone derivative, a flavanone derivative or a pharmaceutically acceptable salt thereof as active ingredients of the complex, mixed or combined preparation.

The biguanide-based compound according to the present invention may be selected from the group consisting of metformin, phenformin, buformin and biguanide.

The flavone, hydroxyflavone, flavanone, flavone derivative, hydroxyflavone derivative, or flavanone derivative according to the present invention may be selected from the group consisting of
2′-hydroxyflavone,
3-hydroxyflavone (flavonol),
3′-hydroxyflavone,
4′-hydroxyflavone,
5-hydroxyflavone (primuliten),
6-hydroxyflavone,
7-hydroxyflavone,
8-hydroxyflavone,
3′,4′-dihydroxyflavone,
3,6-dihydroxyflavone,
3,7-dihydroxyflavone (resogalangin),
4′,7-dihydroxyflavone,
5,7-dihydroxyflavone (chrysin),
7-O-acetyl chrysin (monoacetyl chrysin),
5,7-di-O-methoxy chrysin (dimethyl chrysin),
5,7-di-O-acetyl chrysin (diacetyl chrysin),
6,7-dihydroxyflavone,
7,4′-dihydroxyflavone,
7,8-dihydroxyflavone,
3,5,7-trihydroxyflavone (galangin),
3,7,4′-trihydroxyflavone (resokaempferol),
4′,5,7-trihydroxyflavanone (naringenin),
5,3′,4′-trihydroxyflavone,
5,6,7-trihydroxyflavone (baicalein),
5,7,2′-trihydroxyflavone,
5,7,4′-trihydroxyflavone (apigenin),
5,7,8-trihydroxyflavone (norwogonin),
7,3′,4′-trihydroxyflavone,
7,8,3′-trihydroxyflavone,
7,8,4′-trihydroxyflavone,
4′,5,7-triacetoxy flavone (apigenin triacetate),
5-hydroxy-4′,7-dimethoxyflavone,
5,7-dimethoxy-4′-hydroxyflavone,
5,4′-dimethoxy-7-hydroxyflavone,
3′,4′,5,7-tetrahydroxyflavone (luteolin),
3,4′,5,7-tetrahydroxyflavone (kaempferol),
5,6,7,4′-tetrahydroxyflavone (scutellarein),
4′,5,6,7,8-pentamethoxyflavone (tangeretin),
5,6,7,3′,4′-pentamethoxyflavone (sinensetin),
5,7,8,3′,4′-pentamethoxyflavone (isosinensetin),
3,3′,4′,5,6,7-hexahydroxyflavone (quercetagetin),
3′,4′,5,6,7,8-hexamethoxyflavone (nobiletin),
4′,5,7-trihydroxy-3′-methoxyflavone (chrysoeriol),
5,7,3′-trihydroxy-4′-methoxyflavone (diosmetin),
4′,5,7-trihydroxy-6-methoxyflavone (hispidulin),
5,7,4′-trihydroxy-3,6,3′-trimethoxyflavone (jaceidin),
3′,4′,7-trihydroxy-6-methoxyflavone (nepetin),
3,5,7,3′,4′-pentahydroxy-6-methoxyflavone (patuletin),
3,4′,5,7-tetrahydroxy-3′,6-dimethoxyflavone (spinacetin),
5,7,4′-trihydroxy-3′,5′-dimethoxyflavone (tricin),
7-O-beta-D-apiofuranosyl-1,2-beta-D-glucosyl-5,7,4′-trihydroxyflavone (apiin),
7-O-beta-D-glucosyl-5,7,4′-trihydroxyflavone (apigetrin),
5,7,3′,4′-flavon-3-ol (quercetin),
7,3′,4′-flavon-3-ol (fisetin),
4′,5-dihydroxy-7-methoxyflavone (genkwanin),
4′,5,7-trihydroxyflavanone-7-rhamnoglucoside (naringin),
5-hydroxy-2-(4-hydroxyphenyl)-4-oxo-4H-chromen-7-yl-2-O-(alpha-L-rhamnopyranosyl)-beta-D-glucopyranoside (rhoifoloside) and
8alpha-L-arabinopyranosyl-6beta-D-glucopyranosyl-5,7-dihydroxy-2-(4-hydroxyphenyl)-4H-1-Benzopyran-4-one (schaftoside).

In the present invention, “prevention” means any action that suppresses the onset of disease or delays the onset of disease by administration of the composition. In the present invention, “improvement” or “treatment” means any action that improves or advantageously changes the symptoms of the disease by administration of the composition.

In the present invention, “administration” means providing a given substance to a patient by any suitable method, and the composition of the present invention may be administered orally or parenterally through all general administration routes as long as it can reach the target tissue. The composition may be administered in a form mounted on any device capable of transporting the active substance to a target cell.

In the present invention, metformin is a compound represented by the following Chemical Formula 1:

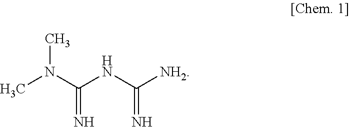

In the present invention, phenformin is a compound represented by the following Chemical Formula 2:

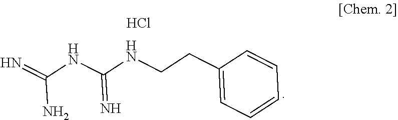 [SMILES: Cl ; N=C(N)NC(=N)NCCc1ccccc1]

In the present invention, buformin is a compound represented by the following Chemical Formula 3:

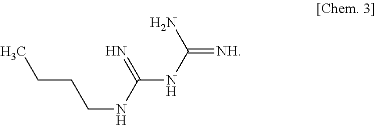

In the present invention, biguanide is a compound represented by the following Chemical Formula 4:

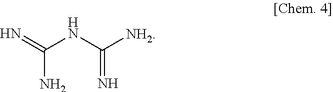

In the present invention, the flavone derivative, hydroxyflavone derivative, and flavanone derivative are compounds represented by the following Chemical Formulas 5 to 63:

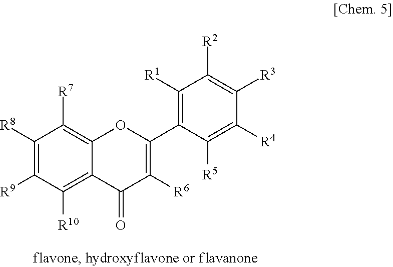

where R¹ to R⁵ are each independently —H, —OH, CₖH₂ₖ₊₁O— or CₖH₂ₖ₊₁COO— (k is an integer from 1 to 5),

R⁶ is —H, —OH or CₘH₂ₘ₊₁O— (m is an integer from 1 to 5), and

R⁷ to R¹⁰ are each independently —H, —OH, CₙH₂ₙ₊₁O— or CₙH₂ₙ₊₁COO— or

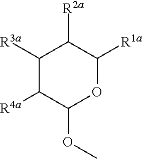

(n is an integer from 1 to 5),

where R^{1a} to R^{4a} are each independently —H, —OH, —CH₂OH,

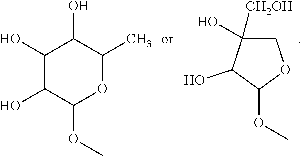

More specifically, the flavone, hydroxyflavone, flavanone, flavone derivative, hydroxyflavone derivative, or flavanone derivative according to the present invention may be selected from compounds represented by the following Chemical Formulas 6 to 63.

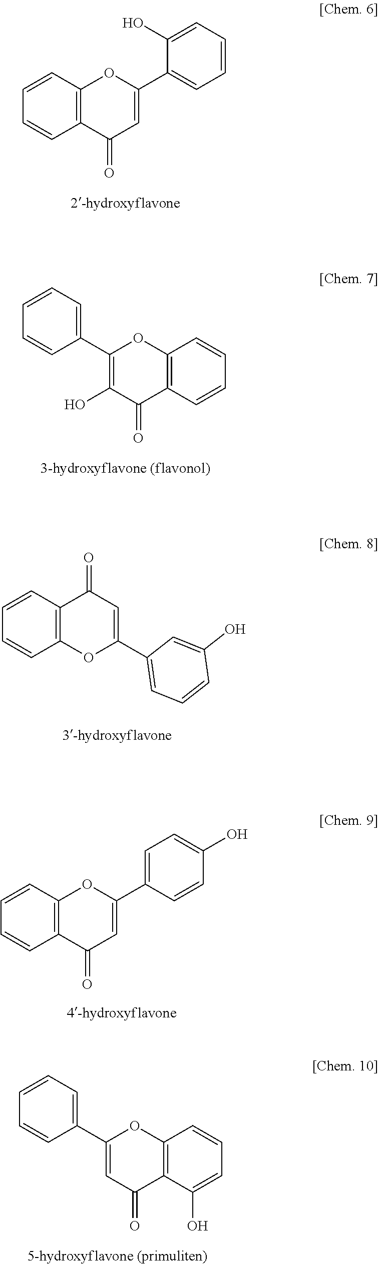 [SMILES: O=c1cc(-c2ccccc2O)oc2ccccc12 ; O=c1c(O)c(-c2ccccc2)oc2ccccc12 ; O=c1cc(-c2cccc(O)c2)oc2ccccc12 ; O=c1cc(-c2ccc(O)cc2)oc2ccccc12 ; O=c1cc(-c2ccccc2)oc2cccc(O)c12]

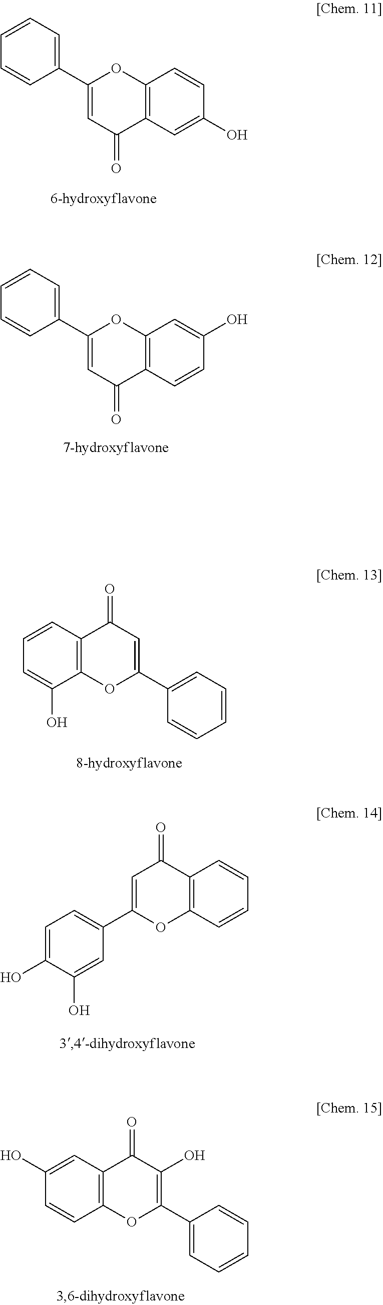 [SMILES: O=c1cc(-c2ccccc2)oc2ccc(O)cc12 ; O=c1cc(-c2ccccc2)oc2cc(O)ccc12 ; O=c1cc(-c2ccccc2)oc2c(O)cccc12 ; O=c1cc(-c2ccc(O)c(O)c2)oc2ccccc12 ; O=c1c(O)c(-c2ccccc2)oc2ccc(O)cc12]

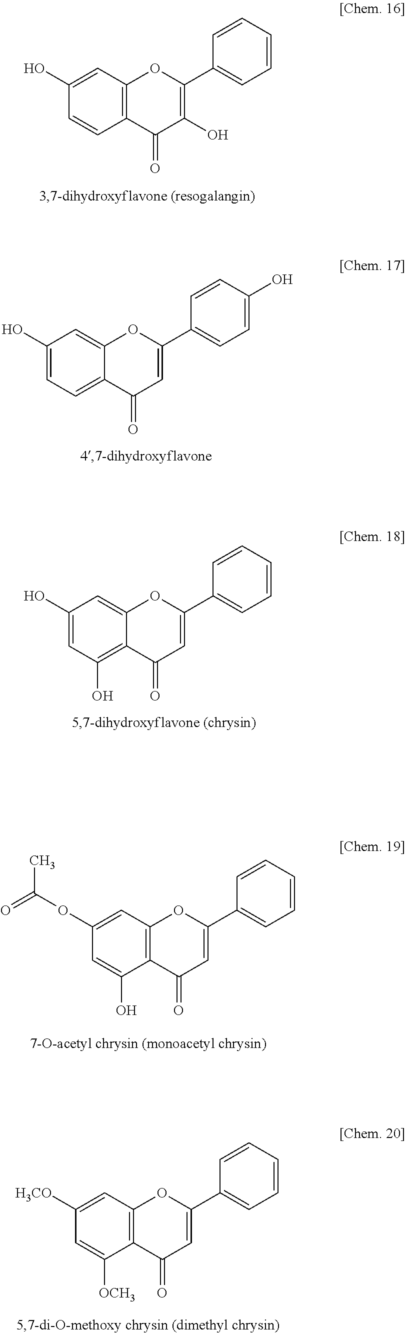 [SMILES: O=c1c(O)c(-c2ccccc2)oc2cc(O)ccc12 ; O=c1cc(-c2ccc(O)cc2)oc2cc(O)ccc12 ; O=c1cc(-c2ccccc2)oc2cc(O)cc(O)c12 ; CC(=O)Oc1cc(O)c2c(=O)cc(-c3ccccc3)oc2c1 ; COc1cc(OC)c2c(=O)cc(-c3ccccc3)oc2c1]

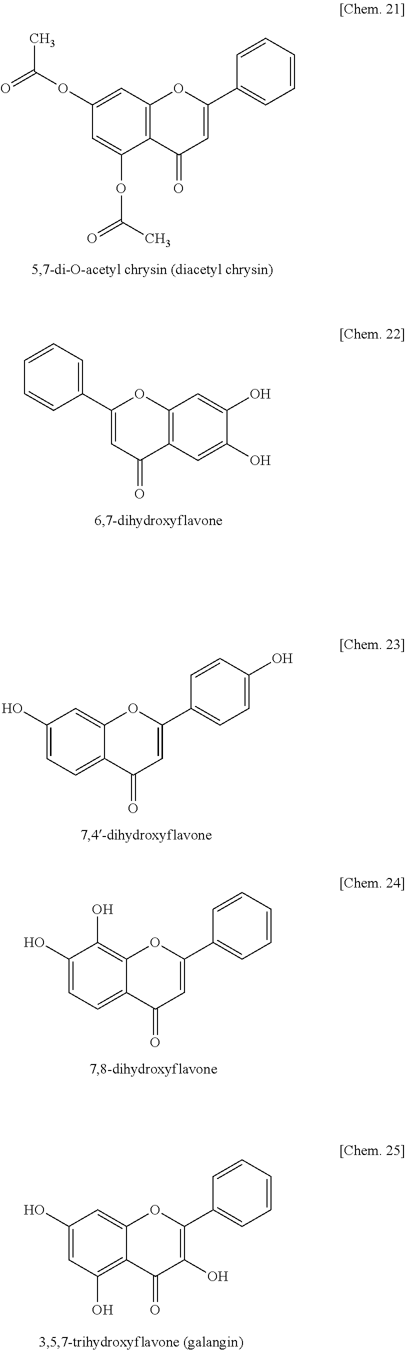 [SMILES: CC(=O)Oc1cc(OC(C)=O)c2c(=O)cc(-c3ccccc3)oc2c1 ; O=c1cc(-c2ccccc2)oc2cc(O)c(O)cc12 ; O=c1cc(-c2ccc(O)cc2)oc2cc(O)ccc12 ; O=c1cc(-c2ccccc2)oc2c(O)c(O)ccc12 ; O=c1c(O)c(-c2ccccc2)oc2cc(O)cc(O)c12]

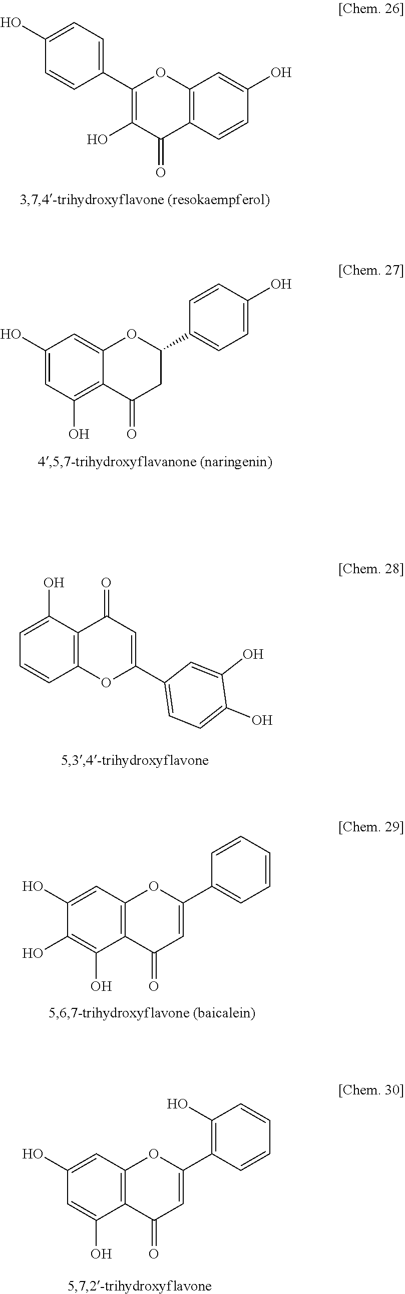 [SMILES: O=c1c(O)c(-c2ccc(O)cc2)oc2cc(O)ccc12 ; O=C1C[C@@H](c2ccc(O)cc2)Oc2cc(O)cc(O)c21 ; O=c1cc(-c2ccc(O)c(O)c2)oc2cccc(O)c12 ; O=c1cc(-c2ccccc2)oc2cc(O)c(O)c(O)c12 ; O=c1cc(-c2ccccc2O)oc2cc(O)cc(O)c12]

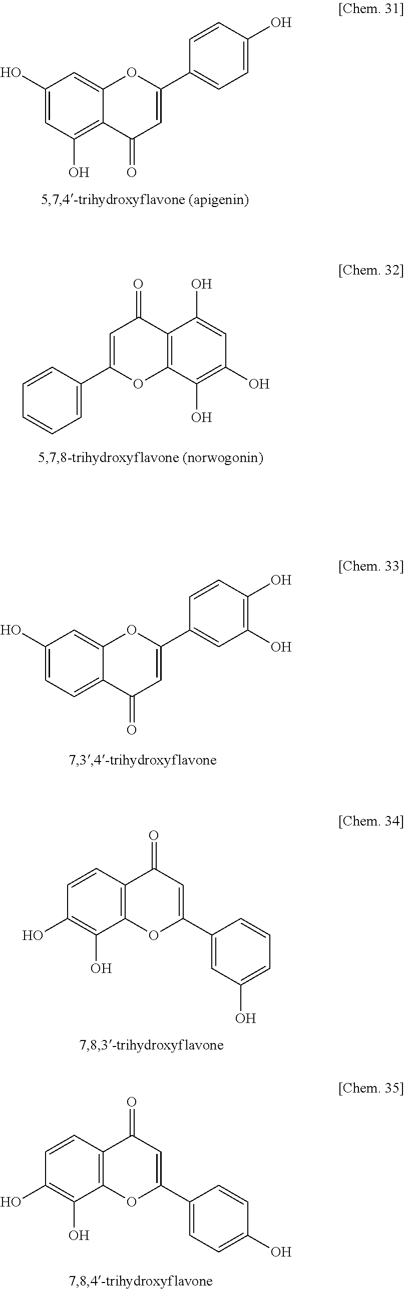 [SMILES: O=c1cc(-c2ccc(O)cc2)oc2cc(O)cc(O)c12 ; O=c1cc(-c2ccccc2)oc2c(O)c(O)cc(O)c12 ; O=c1cc(-c2ccc(O)c(O)c2)oc2cc(O)ccc12 ; O=c1cc(-c2cccc(O)c2)oc2c(O)c(O)ccc12 ; O=c1cc(-c2ccc(O)cc2)oc2c(O)c(O)ccc12]

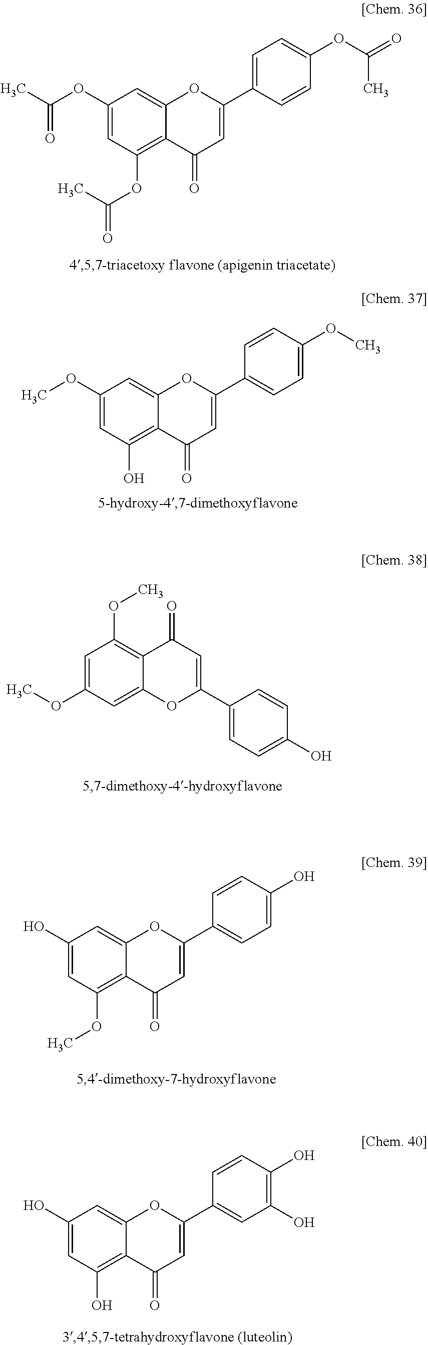

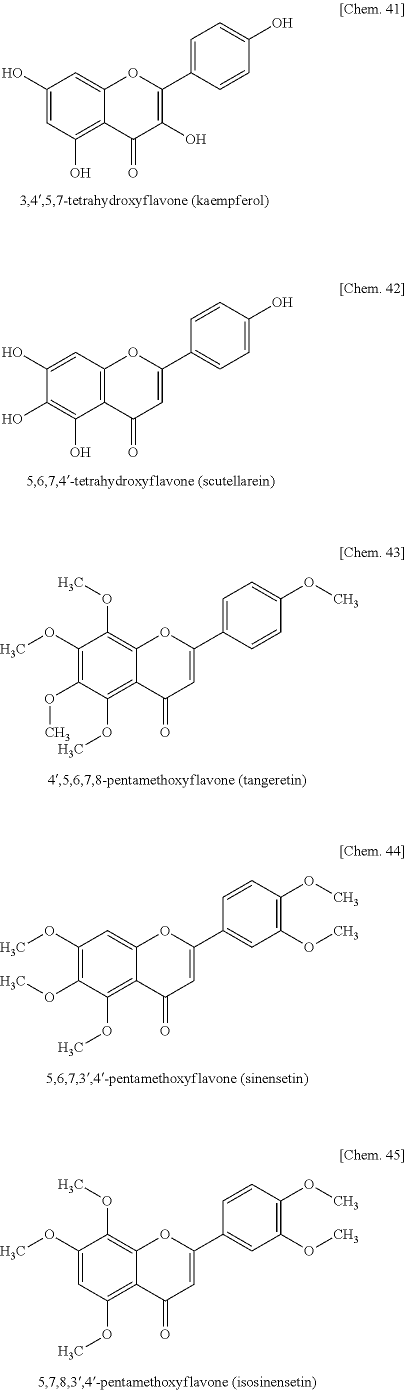

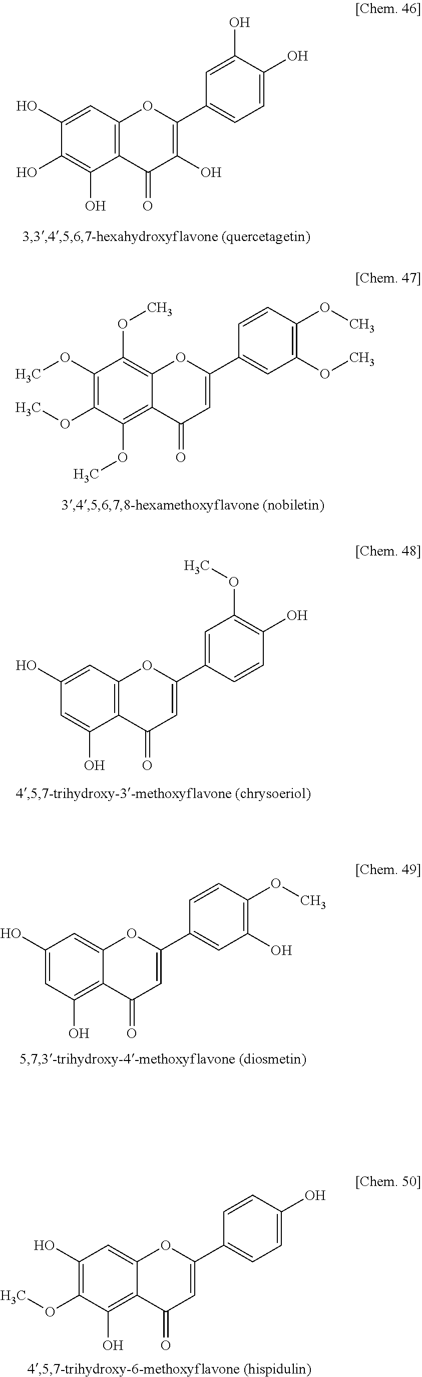

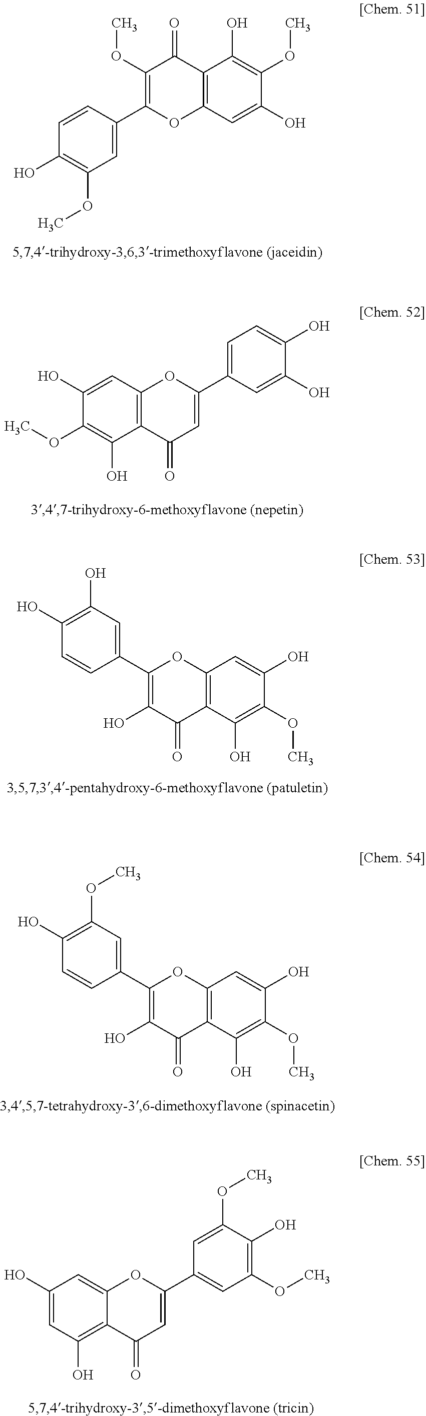

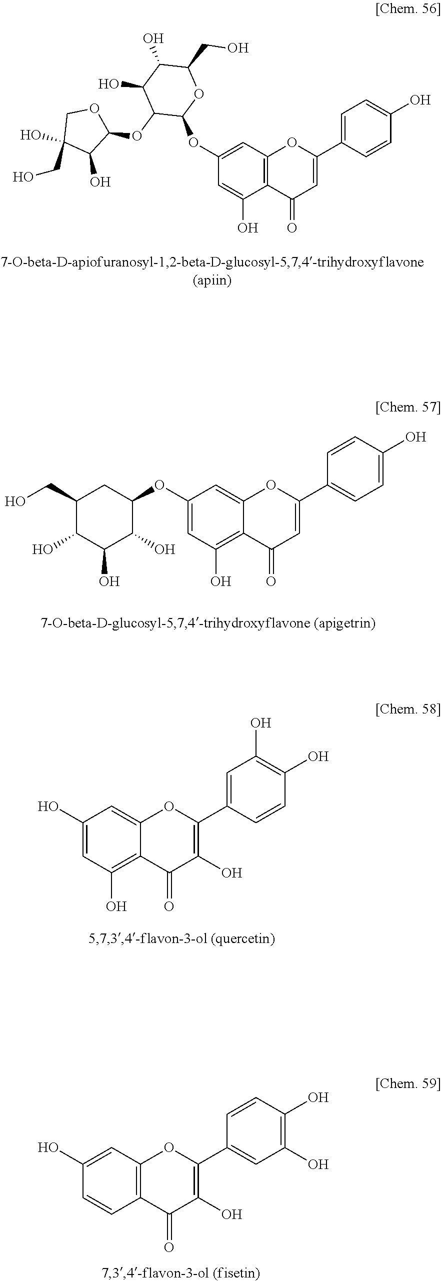 [SMILES: O=c1cc(-c2ccc(O)cc2)oc2cc(O[C@@H]3O[C@H](CO)[C@@H](O)[C@H](O)C3O[C@@H]3OC[C@](O)(CO)[C@@H]3O)cc(O)c12 ; O=c1cc(-c2ccc(O)cc2)oc2cc(O[C@@H]3C[C@H](CO)[C@@H](O)[C@H](O)[C@H]3O)cc(O)c12 ; O=c1c(O)c(-c2ccc(O)c(O)c2)oc2cc(O)cc(O)c12 ; O=c1c(O)c(-c2ccc(O)c(O)c2)oc2cc(O)ccc12]

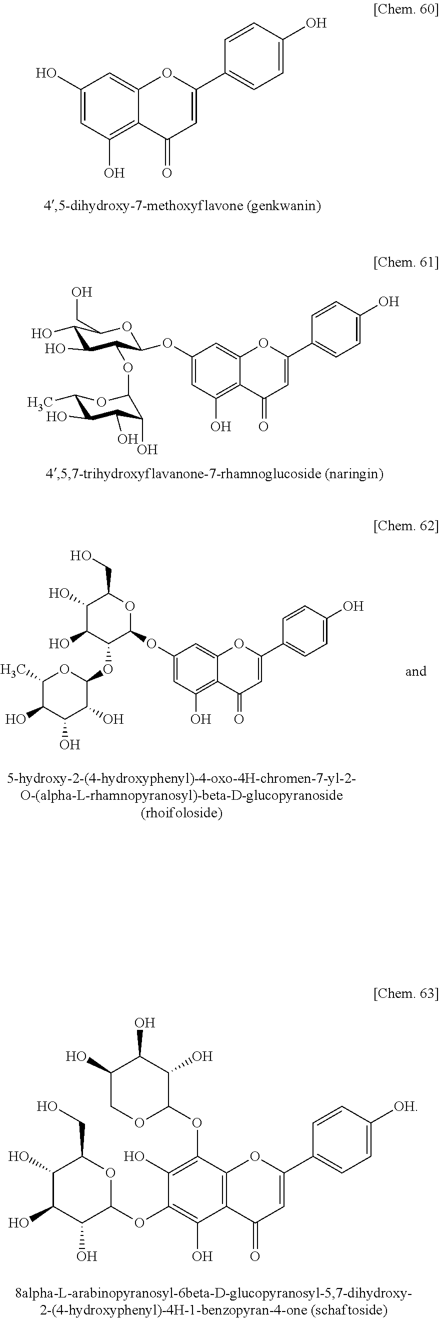

In an aspect of the present invention, the concentration of the biguanide-based compound or a pharmaceutically acceptable salt thereof may be from 0.1 mM to 100 mM, and the concentration of the flavone, hydroxyflavone, flavanone, flavone derivative, hydroxyflavone derivative, flavanone derivative or a pharmaceutically acceptable salt thereof may be from 0.001 μM to 10 mM.

In an aspect of the present invention, the contents of the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof in the medicament of the present invention may be appropriately selected depending on the form of the preparation, and the like.

In an aspect of the present invention, in a case where the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof are formulated into a single preparation, the content of the biguanide-based compound or a pharmaceutically acceptable salt thereof is generally from about 0.01 to about 99.99 wt %, specifically from about 0.01 to about 90 wt %, preferably from about 0.1 to about 90 wt %, more preferably from about 0.1 to about 80 wt %, still more preferably from about 0.1 to about 70 wt % with respect to the entire preparation, and the content of the flavone, hydroxyflavone, flavanone, flavone derivative, hydroxyflavone derivative, flavanone derivative or a pharmaceutically acceptable salt thereof is generally from about 0.01 to about 99.99 wt %, specifically from about 0.01 to about 90 wt %, preferably from about 0.1 to about 80 wt %, more preferably from about 0.1 to about 70 wt %, and still more preferably from about 0.1 to about 60 wt % with respect to the entire preparation. Meanwhile, in the case of being blended as a single preparation, the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof may be blended in the medicament of the present invention at a weight ratio of 1:0.0000001 to 10. In the case of being blended as a single preparation, the content of additives such as carriers in the medicament of the present invention is variable, but may be generally from about 1 to about 99.00 wt %, specifically from about 1 to about 90 wt %, preferably from about 10 to about 90 wt %, more preferably from about 10 to 80 wt %, and still more preferably from about 10 to about 70 wt % with respect to the entire preparation.

In an aspect of the present invention, in a case where the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof are formulated separately and used in combination, the content of the biguanide-based compound or a pharmaceutically acceptable salt thereof in the preparation containing the biguanide-based compound or a pharmaceutically acceptable salt thereof is generally from about 0.01 to about 99.99 wt %, specifically from about 0.1 to about 99.99 wt %, preferably from about 0.1 to about 90 wt %, more preferably from about 0.1 to about 80 wt %, and still more preferably from about 1 to about 80 wt % with respect to the preparation containing the corresponding ingredient. The content of the flavone, hydroxyflavone, flavanone, flavone derivative, hydroxyflavone derivative, flavanone derivative or a pharmaceutically acceptable salt thereof in the preparation containing the flavone, hydroxyflavone, flavanone, flavone derivative, hydroxyflavone derivative, flavanone derivative or a pharmaceutically acceptable salt thereof may be generally from about 0.01 to about 99.99 wt %, specifically from about 0.1 to about 99.9 wt %, preferably from about 0.1 to about 90 wt %, and more preferably from about 0.1 to about 80 wt % with respect to the preparation containing the corresponding ingredient. Meanwhile, in a case where the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof are formulated separately and used in combination, the content of additives such as carriers is variable, but may be generally from about 1 to 99.00 wt %, specifically from about 1 to about 90 wt %, preferably from about 10 to about 90 wt %, more preferably from about 10 to 80 wt %, and still more preferably from about 10 to about 70 wt % with respect to each preparation containing the corresponding ingredient.

In an aspect of the present invention, the cancer may be selected from the group consisting of (A) breast cancers, including (1) ductal carcinoma, including ductal carcinoma in situ (DCIS) (comedocarcinoma, cribriform, papillary, micropapillary), invasive ductal carcinoma (IDC), ductal carcinoma, mucinous (colloidal) carcinoma, papillary carcinoma, metaplastic carcinoma and inflammatory carcinoma; (2) lobular carcinomas, including lobular carcinoma in situ (LCIS) and invasive lobular carcinoma; and (3) Paget's disease of the nipple; (B) cancers of the female reproductive system, including (1) cancers of the cervix, including cervical intraepithelial neoplasia (grade I), cervical intraepithelial neoplasia (grade II), cervical intraepithelial neoplasia (grade III) (squamous cell carcinoma in situ), keratinizing squamous cell carcinoma, nonkeratinizing squamous cell carcinoma, verrucous carcinoma, adenocarcinoma in situ, adenocarcinoma in situ, endometrial type carcinoma, endometrioid adenocarcinoma, clear cell adenocarcinoma, adenoepithelioma, adenoid cystic carcinoma, small cell carcinoma and undifferentiated carcinoma; (2) cancers of the uterine body, including endometrioid carcinoma, adenocarcinoma, adenoacanthoma (adenocarcinoma with squamous metaplasia), adenoepithelioma (mixed adenocarcinoma and squamous cell carcinoma), mucinous adenocarcinoma, serous adenocarcinoma, clear cell adenocarcinoma, squamous cell adenocarcinoma and undifferentiated adenocarcinoma; (3) cancers of the ovary, including serous cystadenoma, serous cystadenoma, mucinous cystadenoma, mucinous cystadenoma, endometrioid tumor, endometrioid adenocarcinoma, clear cell tumor, clear cell cystadenoma and unclassified tumors; (4) cancers of the vagina, including squamous cell carcinoma and adenocarcinoma; and (5) vulvar cancers, including vulvar intraepithelial neoplasia (grade I), vulvar intraepithelial neoplasia (grade II), vulvar intraepithelial neoplasia (grade III) (squamous cell carcinoma in situ); squamous cell carcinoma, verrucous carcinoma, Paget's disease of the vulva, adenocarcinoma (NOS); basal cell carcinoma (NOS) and Bartholin gland carcinoma; (C) cancers of the male reproductive system, including (1) cancer of the penis, including squamous cell carcinoma; (2) cancers of the prostate, including adenocarcinomas, sarcomas, and transitional cell carcinomas of the prostate; and (3) cancers of the testes, including seminoma tumor, non-seminoma tumor, teratomas, embryonic carcinomas, yolk sac tumor and choriocarcinoma; (D) cancers of the heart system, including sarcomas (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma and squamous cell carcinoma of the pharynx; (F) cancers of the lung, including squamous cell carcinoma (epidermoid carcinoma), a variant of squamous cell carcinoma, spindle cell carcinoma, small cell carcinoma, carcinoma of other cells, carcinoma of the intermediate cell type, complex oat cell carcinoma, adenocarcinoma, acinar adenocarcinoma, papillary adenocarcinoma, bronchoalveolar carcinoma, mucin-producing solid carcinoma, giant cell carcinoma, giant cell carcinoma, clear cell carcinoma and sarcoma; (G) cancers of the gastrointestinal tract, including (1) cancers of the ampulla of vater, including primary adenocarcinoma, carcinoid tumor and lymphoma; (2) cancers of the anal canal, including adenocarcinoma, squamous cell carcinoma and melanoma; (3) cancers of the extrahepatic bile duct, including carcinoma in situ, adenocarcinoma, papillary adenocarcinoma, adenocarcinoma, intestinal type, mucinous adenocarcinoma, clear cell adenocarcinoma, signet ring cell carcinoma, adenoepithelioma, squamous cell carcinoma, small cell (oat cell) carcinoma, undifferentiated carcinoma, carcinoma (NOS), sarcoma and carcinoid tumor; (4) cancers of the colon and rectum, including adenocarcinoma in situ, adenocarcinoma, mucinous adenocarcinoma (colloidal type; >50% mucinous carcinoma), signet ring cell carcinoma (greater than 50% of signet ring cells), squamous cell (epidermoid) carcinoma, adenoepithelioma, small cell (oat cell) carcinoma, undifferentiated carcinoma, carcinoma (NOS), sarcoma, lymphoma and carcinoid tumor; (5) cancers of the esophagus, including squamous cell carcinoma, adenocarcinoma, leiomyosarcoma and lymphoma; (6) cancers of the gallbladder, including adenocarcinoma, adenocarcinoma, bowel type, adenoepithelioma, carcinoma in situ, carcinoma (NOS), clear cell adenocarcinoma, mucinous adenocarcinoma, papillary adenocarcinoma, signet ring cell carcinoma, small cell (oat cell) carcinoma, squamous cell carcinoma and undifferentiated carcinoma; (7) cancers of the lips and oral cavity, including squamous cell carcinoma; (8) cancers of the liver, including liver cancer (hepatocellular carcinoma), cholangiocarcinoma, hepatoblastoma, hemangiosarcoma, hepatocellular adenoma and hemangioma; (9) cancers of the exocrine pancreas, including ductal cell carcinoma, giant cell carcinoma multiforme, giant cell carcinoma, osteoclastoid type, adenocarcinoma, adenoepithelioma, mucinous (colloidal) carcinoma, cystadenoma, acinar cell carcinoma, papillary carcinoma, small cell (oat cell) carcinoma, mixed cell type, carcinoma (NOS), undifferentiated carcinoma, endocrine cell tumor arising from islet cells of Langerhans and carcinoid tumor; (10) cancers of the salivary glands, including acinar (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well-differentiated or low grade) and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenoepithelioma, squamous cell carcinoma, small cell carcinoma, undifferentiated carcinoma, lymphoma, sarcoma and carcinoid tumor; and (12) cancers of the small intestine, including adenocarcinoma, lymphoma, carcinoid tumor, Kaposi's sarcoma, leiomyoma, hemangioma, lipoma, neurofibromatosis and fibroma; (H) cancers of the urinary system, including (1) cancers of the kidney, including renal cell carcinoma, carcinoma of the Bellini collecting duct, adenocarcinoma, papillary carcinoma, tubular carcinoma, granular cell tumor, clear cell carcinoma (adenocarcinoma of the kidney), sarcoma of the kidney and nephroblastoma; (2) cancers of the renal pelvis and ureter, including transitional cell carcinoma, papillary transitional cell carcinoma, squamous cell carcinoma and adenocarcinoma; (3) cancers of the urethra, including transitional cell carcinoma, squamous cell carcinoma and adenocarcinoma; and (4) cancers of the bladder, including carcinoma in situ, transitional urothelial cell carcinoma, papillary transitional cell carcinoma, squamous cell carcinoma, adenocarcinoma, and undifferentiated carcinoma; and (I) cancers of muscles, bones and soft tissues, including (1) cancers of the bone, including (a) osteogenesis: osteosarcoma; (b) chondrogenesis: chondrosarcoma and mesenchymal chondrosarcoma; (c) giant cell tumor, malignant; (d) Ewing's sarcoma; (e) vascular tumors: hemangioendothelioma, hemangiopericytoma and hemangiosarcoma; (f) connective tissue tumors: fibrosarcoma, liposarcoma, malignant mesenchymoma and undifferentiated sarcoma; and (g) other tumors: chordoma and adamantinoma of the long bones; (2) cancers of soft tissues, including alveolar soft part sarcoma, angiosarcoma, epithelioid sarcoma, extraosseous chondrosarcoma, fibrosarcoma, leiomyosarcoma, liposarcoma, malignant fibrous histiocytoma, malignant hemangiopericytoma, malignant mesenchymoma, malignant Schwannoma, rhabdomyosarcoma, synovial sarcoma and sarcoma (NOS); (3) cancers of the nervous system, including cancers of the skull (osteoma, hemangioma, granuloma, xanthoma, and osteitis deformans), cancers of the meninges (meningioma, meningiosarcoma, and gliomatosis), cancers of the brain (astrocytoma, meduloblastoma, glioma, ependymal glioma, germinoma (pineal tumor), glioblastoma multiforme, oligodendrocytoma, schwannoma, retinoblastoma, and congenital tumor), and cancers of the spinal cord (neurofibromatosis, meningioma, glioma, sarcoma); (4) hematologic malignancies, including myeloid leukemia (acute and chronic), acute lymphoblastic leukemia, chronic lymphocytic leukemia, myeloproliferative disease, multiple myeloma; myelodysplastic syndrome, Hodgkin's disease and non-Hodgkin's lymphoma (malignant lymphoma); (5) cancers of the endocrine system, including (a) cancers of the thyroid gland, including papillary carcinoma (including those of the follicular region), follicular carcinoma, medullary carcinoma and undifferentiated (anaplastic) carcinoma; and (b) neuroblastomas, including sympathicogonioma, sympathoblastoma, malignant ganglioneuroma, ganglioneuroblastoma and ganglioneuroma; (6) cancers of the skin, including squamous cell carcinoma, spindle cell squamous cell carcinoma, basal cell carcinoma, adenocarcinoma arising from sweat glands or sebaceous glands, and malignant melanoma; and (7) cancers of the eye, including (a) cancer of the conjunctiva, including carcinoma of the conjunctiva; (b) cancers of the eyelid, including basal cell carcinoma, squamous cell carcinoma, melanoma of the eyelid and sebaceous cell carcinoma; (c) cancers of the lacrimal gland, including adenocarcinoma, adenoid cystic carcinoma, carcinoma in pleomorphic adenoma, mucoepidermoid carcinoma and squamous cell carcinoma; (d) cancers of the uvea, including spindle cell melanoma, mixed cell melanoma and epithelioid cell melanoma; (e) cancers of the orbit, including sarcoma of the orbit, soft tissue tumor, and sarcoma of the bone; and (f) retinoblastoma.

In an aspect of the present invention, the pharmaceutical composition may be formulated into a formulation selected from the group consisting of a tablet, a capsule, an injections, a troche, a powder, a granule, a solution, a suspension, an oral solution, an emulsion, a syrup, a suppository, a vaginal tablet and a pill, but is not limited thereto, and can be formulated in an appropriate formulation as needed.

The present invention also provides a complex, mixed or combined preparation kit for prevention or treatment of cancer, the kit comprising a complex, mixed or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof.

In an aspect of the present invention, in the complex, mixed or combined preparation kit for prevention or treatment of cancer, the kit comprising a complex, mixed or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof, the content and content ratio of each component in the combined preparation and the cancer are the same as those in the description of the pharmaceutical composition for the prevention or treatment of cancer, and specific description thereof refers to the above contents.

In the present invention, the complex, mixed or combined preparation of a biguanide-based compound and/or a flavone, a hydroxyflavone, a flavanone, a flavone derivative, a hydroxyflavone derivative, or a flavanone derivative may be used in the form of a pharmaceutically acceptable salt, and an acid addition salt formed using a pharmaceutically acceptable free acid is useful as the salt. Acid addition salts are obtained from inorganic acids such as hydrochloric acid, nitric acid, phosphoric acid, sulfuric acid, hydrobromic acid, hydroiodic acid, nitrous acid or phosphorous acid and non-toxic organic acids such as aliphatic mono and dicarboxylates, phenyl-substituted alkanoates, hydroxy alkanoates and alkandioates, aromatic acids, and aliphatic and aromatic sulfonic acids. Such pharmaceutically non-toxic salts include sulfates, pyrosulfates, bisulfates, sulfites, bisulfites, nitrates, phosphates, monohydrogen phosphates, dihydrogen phosphates, metaphosphates, pyrophosphate chloride, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butyne-1,4-dioates, hexane-1,6-dioates, benzoates, chlorobenzoates, methylbenzoates, dinitrobenzoates, hydroxybenzoates, methoxybenzoates, phthalates, terephthalates, benzenesulfonates, toluenesulfonates, chlorobenzenesulfonates, xylenesulfonates, phenylacetates, phenylpropionates, phenylbutyrates, citrates, lactates, hydroxybutyrates, glycolates, malates, tartrates, methanesulfonates, propanesulfonates, naphthalene-1-sulfonates, naphthalene-2-sulfonates or mandelates.

The acid addition salt according to the present invention may be produced by a conventional method, for example, by dissolving the compound represented by Chemical Formulas 1 to 63 in an excess aqueous acid solution and precipitating this salt using a water-miscible organic solvent, for example, methanol, ethanol, acetone or acetonitrile. The acid addition salt may also be produced by evaporating the solvent or excess acid from this mixture to dryness, or by performing suction filtration of the precipitated salt.

In addition, a pharmaceutically acceptable metal salt may be produced using a base. An alkali metal or alkaline earth metal salt is obtained, for example, by dissolving the compound in an excess alkali metal hydroxide or alkaline earth metal hydroxide solution, filtering the undissolved compound salt, and evaporating and drying the filtrate. In this case, it is pharmaceutically suitable to prepare a sodium, potassium or calcium salt as the metal salt. A silver salt corresponding thereto is obtained by reacting the alkali metal or alkaline earth metal salt with a suitable silver salt (for example, silver nitrate).

In the case of formulating the composition into a preparation, the preparation is usually manufactured using commonly used diluents or excipients such as fillers, extenders, binders, wetting agents, disintegrants, and surfactants.

A solid preparation for oral administration includes tablets, pills, powders, granules, capsules, troches and the like, and such a solid preparation is prepared by mixing one or more compounds represented by Chemical Formulas 1 to 63 of the present invention with at least one or more excipients, for example, starch, calcium carbonate, sucrose or lactose or gelatin. In addition to simple excipients, lubricants such as magnesium stearate and talc are also used. Liquid preparations for oral administration include suspensions, oral solutions, emulsions, or syrups, and may contain various excipients, for example, wetting agents, sweetening agents, fragrances, and preservatives in addition to water and liquid paraffin, which are commonly used simple diluents.

Preparations for parenteral administration contain sterile aqueous solutions, non-aqueous solvents, suspension solvents, emulsions, lyophilized preparations, suppositories, and the like.

As non-aqueous solvents and suspension solvents, propylene glycol, polyethylene glycol, vegetable oils such as olive oil, injectable esters such as ethyl oleate, and the like may be used. As the base of suppositories, witepsol, macrogol, tween 61, cacao butter, laurin oil, glycerol, gelatin and the like may be used.

The present invention also provides a method for preventing or improving cancer, the method comprising administering a pharmaceutically effective amount of a complex, mixed, or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof to an individual.

The administration of the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof according to the present invention in a complex, mixed or combined manner can be usefully utilized to prevent or improve cancer since cancer cell-specific and synergistic anticancer activity is exhibited by the administration.

The present invention also provides a method for treating cancer, the method comprising administering a pharmaceutically effective amount of a complex, mixed, or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof to an individual.

The administration of the complex, mixed, or combined preparation of the biguanide-based compound or a pharmaceutically acceptable salt thereof; and the flavone, hydroxyflavone, flavanone, flavone derivative, hydroxyflavone derivative, flavanone derivative or a pharmaceutically acceptable salt thereof according to the present invention in a complex, mixed or combined manner can be usefully utilized to treat cancer since cancer cell-specific anticancer activity is exhibited by the administration.

The present invention also provides use of a mixed or combined preparation to be used as a pharmaceutical composition for prevention or treatment of cancer, the mixed or combined preparation comprising a first component including a biguanide-based compound or a pharmaceutically acceptable salt thereof; and a second component including a flavone, a hydroxyflavone, a flavanone, a flavone derivative, a hydroxyflavone derivative, a flavanone derivative or a pharmaceutically acceptable salt thereof as active ingredients.

The present invention also provides use of a mixed or combined preparation to be used as health food for prevention or improvement of cancer, the mixed or combined preparation comprising a first component including a biguanide-based compound or a pharmaceutically acceptable salt thereof; and a second component including a flavone, a hydroxyflavone, a flavanone, a flavone derivative, a hydroxyflavone derivative, a flavanone derivative or a pharmaceutically acceptable salt thereof as active ingredients.

The present invention also provides a first component including a biguanide-based compound or a pharmaceutically acceptable salt thereof; and a second component including a flavone, a hydroxyflavone, a flavanone, a flavone derivative, a hydroxyflavone derivative, a flavanone derivative or a pharmaceutically acceptable salt thereof for manufacture of a mixed or combined preparation for preventing or treating cancer.

The present invention also provides a first component including a biguanide-based compound or a pharmaceutically acceptable salt thereof; and a second component including a flavone, a hydroxyflavone, a flavanone, a flavone derivative, a hydroxyflavone derivative, a flavanone derivative or a pharmaceutically acceptable salt thereof for manufacture of a mixed or combined health food for preventing or improving cancer.

In the present invention, the “pharmaceutically effective amount” refers to an amount sufficient to treat a disease at an effective benefit/risk ratio applicable to medical treatment, and the effective dose level may be determined depending on factors including the kind of patient's disease, severity, drug activity, drug sensitivity, administration time, administration route and excretion rate, treatment period, and concomitant drugs and other factors well known in the medical arts. The composition of the present invention may be administered as a blended individual therapeutic agent, or may be administered with other therapeutic agents in a complex, mixed or combined manner, may be administered sequentially or simultaneously with conventional therapeutic agents, and may be administered single time or multiple times. It is important to administer the composition in a minimum amount to obtain the maximum effect without side effects in consideration of all of the factors mentioned above, and the minimum amount may be easily determined by those skilled in the art.

Specifically, the effective amount of the compound according to the present invention may vary depending on the age, sex, and weight of the patient, and the compound may be administered by generally from 0.001 mg to 100 mg, preferably from 0.005 mg to 10 mg per 1 kg of body weight daily or every other day or in divided doses 1 to 3 times a day. However, the dosage is not intended to limit the scope of the present invention in any way since the effective amount may increase or decrease depending on administration route, severity of disease, sex, weight, age and the like.

In an aspect of the present invention, the first component that is a biguanide-based compound or a pharmaceutically acceptable salt thereof; and the second component that is a flavone, a hydroxyflavone, a flavanone, a flavone derivative, a hydroxyflavone derivative, a flavanone derivative or a pharmaceutically acceptable salt thereof are administered to a subject in need of cancer treatment in a blended, complex, mixed or combined manner. Various cancers, including the above-mentioned cancer diseases, can be treated by the method according to the present invention.

In specific Examples of the present invention, in order to confirm the anticancer activity of the complex, mixed or combined preparations of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof, the present inventors have treated breast cancer cells, colon cancer cells, lung cancer cells, prostate cancer cells, pancreatic cancer cells and normal cells with metformin, which is a biguanide-based compound, and a flavone, a hydroxyflavone, a flavanone, a flavone derivative, a hydroxyflavone derivative, or a flavanone derivative singly or in a complex, mixed or combined manner, then conducted MTT analysis, and as a result, confirmed that a growth inhibitory effect is exerted in cancer cells while no change is observed in normal cells. In addition, it has been confirmed that the ceiling effect showing significantly higher growth inhibition is exerted in a case where the cancer cells are treated with metformin and a flavone, a hydroxyflavone, a flavanone, a flavone derivative, a hydroxyflavone derivative, or a flavanone derivative in a complex, mixed, or combined manner compared to a case where the cancer cells are treated with each of these singly.

In the present invention, the subject is a mammal in need of cancer treatment. Typically the subject is a human cancer patient. In an aspect of the present invention, the subject may be a non-human mammal, such as a non-human primate, an animal used in the model system (for example, a mouse and a rat used for screening, characterization and evaluation of pharmaceuticals), and other mammals, for example a primate animal such as a rabbit, guinea pig, hamster, dog, cat, chimpanzee, gorilla, or monkey.

In an aspect of the present invention, the pharmaceutical composition may be used singly or concurrently with surgery, hormone therapy, drug therapy, and biological response modifiers for the treatment of cancer patients.

The present invention also provides use of a complex, mixed or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof to be used as a pharmaceutical composition for prevention or treatment of cancer. The biguanide-based compound is metformin or a pharmaceutically acceptable salt thereof, phenformin or a pharmaceutically acceptable salt thereof, buformin or a pharmaceutically acceptable salt thereof, or a biguanide or a pharmaceutically acceptable salt thereof.

The present invention also provides use of a complex, mixed or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof to be used as a pharmaceutical composition for prevention or treatment of cancer. The biguanide-based compound is metformin, phenformin, buformin, biguanide, or a pharmaceutically acceptable salt thereof.

The present invention also provides use of a complex, mixed or combined preparation of a biguanide-based compound or a pharmaceutically acceptable salt thereof; and a flavone, a hydroxyflavone, a flavanone, a flavone derivative, a hydroxyflavone derivative, a flavanone derivative or a pharmaceutically acceptable salt thereof to be used as health food for prevention or improvement of cancer.

### EXAMPLES

Hereinafter, the present invention will be described in more detail through Examples and Experimental Examples. However, the following Examples and Experimental Examples are intended to help the understanding of the present invention, but are not intended to limit the scope of the present invention.

### <Example 1> Confirmation of Anticancer Activity of Biguanide-Based Compound and Hydroxyflavone Derivative

### <1-1> Confirmation of Anticancer Activity of Metformin and 5,7,4′-trihydroxyflavone (Apigenin) Against Breast Cancer

In order to investigate the anticancer activity of metformin and 5,7,4′-trihydroxyflavone, breast cancer cells were treated with metformin and 5,7,4′-trihydroxyflavone and MTT (3-(4,5-dimethylthiazol-2-yl)-2,5-diphenyltetrazolium bromide) analysis was conducted to examine growth inhibition.

Specifically, the MCF-7 cell line, which was a breast cancer cell line, was incubated in a 100 mm culture dish using DMEM-10% FBS under 5% CO₂ at 37° C., inoculated into each well of a 96-well plate at 20% confluence, and incubated for 24 hours. The incubated MCF-7 cell line was treated with metformin at a concentration of 5 mM and 5,7,4′-trihydroxyflavone at a concentration of 0.01, 0.1, 1, or 10 μM, and incubated in a CO₂ incubator for 24, 48, or 72 hours. The culture medium was removed from each well, 100 μl of a fresh culture medium was added, and 10 μl of a 12 mM MTT stock solution (5 mg MTT/PBS) was added, and incubation was performed at 37° C. for 2 hours. Thereafter, 100 μl of SDS-HCl solution (1 g SDS/10 ml 0.01 M HCl), which was a reaction terminating solution, was added, incubation was performed at 37° C. for 4 hours, and OD was measured at 570 nM using a microplate leader. Percentage (%) growth inhibition was calculated by comparing the OD thus measured to the OD of the cells not treated with drugs (FIG. 1A). For the normal cell control group, MTT analysis was conducted in the same manner as that described above using the WISH (human normal epithelial cells) cell line. In the case of the normal cell control group, the cell line was treated with drugs and incubated for 24, 48, 72, or 96 hours (FIG. 1B).

As a result, as illustrated in FIGS. 1A and 1B, it has been confirmed that a ceiling effect showing significantly higher growth inhibition is exerted in a case where breast cancer cells are treated with 5 mM metformin and 0.01, 0.1, 1, or 10 μM 5,7,4′-trihydroxyflavone in combination compared to a case where breast cancer cells are treated with 5 mM metformin or 0.01, 0.1, 1, or 10 μM 5,7,4′-trihydroxyflavone singly (FIG. 1A). On the other hand, the growth inhibitory activity is as weak as 0% to 8% when human normal epithelial cells are treated with 5 mM metformin and 0.01, 0.1, 1, or 10 μM 5,7,4′-trihydroxyflavone in combination, and it has been thus confirmed that the combined treatment with metformin and 5,7,4′-trihydroxyflavone significantly inhibits the growth of cancer cells but hardly affects normal cells (FIG. 1B).

### <1-2> Confirmation of Anticancer Activity of Metformin and 5,7,4′-trihydroxyflavone Against Colon Cancer

In order to investigate the anticancer activity of metformin and 5,7,4′-trihydroxyflavone, colon cancer cells were treated with metformin and 5,7,4′-trihydroxyflavone and MTT analysis was conducted to examine growth inhibition.

Specifically, MTT analysis was conducted in the same manner as that described in Example <1-1> using the HCT 116 and Caco-2 cell lines, which were colon cancer cell lines. At this time, the cell lines were treated with drugs and incubated for 24, 48, 72, or 96 hours.

As a result, as illustrated in FIGS. 2 and 3, it has been confirmed that a ceiling effect showing significantly higher growth inhibition is exerted in a case where colon cancer cells are treated with 5 mM metformin and 0.01, 0.1, 1, or 10 μM 5,7,4′-trihydroxyflavone in combination compared to a case where colon cancer cells are treated with 5 mM metformin or 0.01, 0.1, 1, or 10 μM 5,7,4′-trihydroxyflavone singly (FIGS. 2 and 3).

### <1-3> Confirmation of Anticancer Activity of Metformin and 5,7,4′-trihydroxyflavone Against Lung Cancer

In order to investigate the anticancer activity of metformin and 5,7,4′-trihydroxyflavone, lung cancer cells were treated with metformin and 5,7,4′-trihydroxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 24, 48, 72, or 96 hours.

As a result, as illustrated in FIG. 4, it has been confirmed that a ceiling effect showing significantly higher growth inhibition is exerted in a case where lung cancer cells are treated with 5 mM metformin and 0.01, 0.1, 1, or 10 μM 5,7,4′-trihydroxyflavone in combination compared to a case where lung cancer cells are treated with 5 mM metformin or 0.01, 0.1, 1, or 10 μM 5,7,4′-trihydroxyflavone singly (FIG. 4).

### <1-4> Confirmation of Anticancer Activity of Metformin and 5,7,4′-trihydroxyflavone Against Prostate Cancer

In order to investigate the anticancer activity of metformin and 5,7,4′-trihydroxyflavone, prostate cancer cells were treated with metformin and 5,7,4′-trihydroxyflavone and MTT analysis was conducted to examine growth inhibition.

Specifically, MTT analysis was conducted in the same manner as that described in Example <1-1> using the DU145 and LNCaP prostate cell lines, which were prostate cancer cell lines. At this time, the cell lines were treated with drugs and incubated for 24, 48, 72, or 96 hours.

As a result, as illustrated in FIGS. 5 and 6, it has been confirmed that a ceiling effect showing significantly higher growth inhibition is exerted in a case where prostate cancer cells are treated with 5 mM metformin and 0.01, 0.1, 1, or 10 μM 5,7,4′-trihydroxyflavone in combination compared to a case where prostate cancer cells are treated with 5 mM metformin or 0.01, 0.1, 1, or 10 μM 5,7,4′-trihydroxyflavone singly (FIGS. 5 and 6).

### <1-5> Confirmation of Anticancer Activity of Metformin and 5,7,4′-trihydroxyflavone Against Pancreatic Cancer

In order to investigate the anticancer activity of metformin and 5,7,4′-trihydroxyflavone, pancreatic cancer cells were treated with metformin and 5,7,4′-trihydroxyflavone and MTT analysis was conducted to examine growth inhibition.

Specifically, MTT analysis was conducted in the same manner as that described in Example <1-1> using the AsPC-1 and MIA Paca-2 cell lines, which were pancreatic cancer cell lines. At this time, the cell lines were treated with drugs and incubated for 24, 48, 72, or 96 hours.

As a result, as illustrated in FIGS. 7 and 8, it has been confirmed that a ceiling effect showing significantly higher growth inhibition is exerted in a case where pancreatic cancer cells are treated with 5 mM metformin and 0.01, 0.1, 1, or 10 μM 5,7,4′-trihydroxyflavone in combination compared to a case where pancreatic cancer cells are treated with 5 mM metformin or 0.01, 0.1, 1, or 10 μM 5,7,4′-trihydroxyflavone singly (FIGS. 7 and 8).

Through the above results, it has been confirmed that the growth of normal cells is hardly affected but a superior effect of inhibiting the growth of cancer cells is exerted when combined treatment with metformin and 5,7,4′-trihydroxyflavone or a salt thereof is performed.

### <Example 2> Confirmation of Anticancer Activity of Biguanide-Based Compound and Hydroxyflavone Derivative

### <2-1> Confirmation of Anticancer Activity of Phenformin and 5,7,4′-trihydroxyflavone (Apigenin) Against Breast Cancer

In order to investigate the anticancer activity of phenformin and 5,7,4′-trihydroxyflavone, breast cancer cells were treated with phenformin and 5,7,4′-trihydroxyflavone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 9A, it has been confirmed that a ceiling effect showing significantly higher growth inhibition is exerted in a case where breast cancer cells are treated with 10, 100, or 1000 μM phenformin and 20 μM 5,7,4′-trihydroxyflavone in combination compared to a case where breast cancer cells are treated with 10, 100, or 1000 μM phenformin or 20 μM 5,7,4′-trihydroxyflavone singly (FIG. 9A). On the other hand, the growth inhibitory activity is 0% to 28% when human normal epithelial cells are treated with 10, 100, or 1000 μM phenformin and 20 μM 5,7,4′-trihydroxyflavone in combination, and it has been thus confirmed that the combined treatment with phenformin and 5,7,4′-trihydroxyflavone significantly inhibits the growth of cancer cells but slightly affects normal cells (FIG. 9B).

### <2-2> Confirmation of Anticancer Activity of Phenformin and 5,7,4′-trihydroxyflavone Against Colon Cancer

In order to investigate the anticancer activity of phenformin and 5,7,4′-trihydroxyflavone, colon cancer cells were treated with phenformin and 5,7,4′-trihydroxyflavone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 10, it has been confirmed that a ceiling effect showing significantly higher growth inhibition is exerted in a case where colon cancer cells are treated with 10, 100, or 1000 μM phenformin and 20 μM 5,7,4′-trihydroxyflavone in combination compared to a case where colon cancer cells are treated with 10, 100, or 1000 μM phenformin or 20 μM 5,7,4′-trihydroxyflavone singly (FIG. 10).

### <2-3> Confirmation of Anticancer Activity of Phenformin and 5,7,4′-trihydroxyflavone Against Lung Cancer

In order to investigate the anticancer activity of phenformin and 5,7,4′-trihydroxyflavone, lung cancer cells were treated with phenformin and 5,7,4′-trihydroxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 11, it has been confirmed that a ceiling effect showing significantly higher growth inhibition is exerted in a case where lung cancer cells are treated with 10, 100, or 1000 μM phenformin and 20 μM 5,7,4′-trihydroxyflavone in combination compared to a case where lung cancer cells are treated with 10, 100, or 1000 μM phenformin or 20 μM 5,7,4′-trihydroxyflavone singly (FIG. 11).

### <2-4> Confirmation of Anticancer Activity of Phenformin and 5,7,4′-trihydroxyflavone Against Prostate Cancer

In order to investigate the anticancer activity of phenformin and 5,7,4′-trihydroxyflavone, prostate cancer cells were treated with phenformin and 5,7,4′-trihydroxyflavone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 12, it has been confirmed that a ceiling effect showing significantly higher growth inhibition is exerted in a case where prostate cancer cells are treated with 10, 100, or 1000 μM phenformin and 20 μM 5,7,4′-trihydroxyflavone in combination compared to a case where prostate cancer cells are treated with 10, 100, or 1000 μM phenformin or 20 μM 5,7,4′-trihydroxyflavone singly (FIG. 12).

### <2-5> Confirmation of Anticancer Activity of Phenformin and 5,7,4′-trihydroxyflavone Against Pancreatic Cancer

In order to investigate the anticancer activity of phenformin and 5,7,4′-trihydroxyflavone, pancreatic cancer cells were treated with phenformin and 5,7,4′-trihydroxyflavone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 13, it has been confirmed that a ceiling effect showing significantly higher growth inhibition is exerted in a case where pancreatic cancer cells are treated with 10, 100, or 1000 μM phenformin and 20 μM 5,7,4′-trihydroxyflavone in combination compared to a case where pancreatic cancer cells are treated with 10, 100, or 1000 μM phenformin or 20 μM 5,7,4′-trihydroxyflavone singly (FIG. 13).

Through the above results, it has been confirmed that the growth of normal cells is slightly affected but a superior effect of inhibiting the growth of cancer cells is exerted when combined treatment with phenformin and 5,7,4′-trihydroxyflavone or a salt thereof is performed.

### <Example 3> Confirmation of Anticancer Activity of Biguanide-Based Compound and Hydroxyflavone Derivative

### <3-1> Confirmation of Anticancer Activity of Buformin and 5,7,4′-trihydroxyflavone (Apigenin) Against Breast Cancer

In order to investigate the anticancer activity of buformin and 5,7,4′-trihydroxyflavone, breast cancer cells were treated with buformin and 5,7,4′-trihydroxyflavone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 14A, it has been confirmed that a ceiling effect showing significantly higher growth inhibition is exerted in a case where breast cancer cells are treated with 10, 100, or 1000 μM buformin and 20 μM 5,7,4′-trihydroxyflavone in combination compared to a case where breast cancer cells are treated with 10, 100, or 1000 μM buformin or 20 μM 5,7,4′-trihydroxyflavone singly (FIG. 14A). On the other hand, the growth inhibitory activity is 0% to 16% when human normal epithelial cells are treated with 10, 100, or 1000 μM buformin and 20 μM 5,7,4′-trihydroxyflavone in combination, and it has been thus confirmed that the combined treatment with buformin and 5,7,4′-trihydroxyflavone significantly inhibits the growth of cancer cells but slightly affects normal cells (FIG. 14B).

### <3-2> Confirmation of Anticancer Activity of Buformin and 5,7,4′-trihydroxyflavone Against Colon Cancer

In order to investigate the anticancer activity of buformin and 5,7,4′-trihydroxyflavone, colon cancer cells were treated with buformin and 5,7,4′-trihydroxyflavone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 15, it has been confirmed that a ceiling effect showing significantly higher growth inhibition is exerted in a case where colon cancer cells are treated with 10, 100, or 1000 μM buformin and 20 μM 5,7,4′-trihydroxyflavone in combination compared to a case where colon cancer cells are treated with 10, 100, or 1000 μM buformin or 20 μM 5,7,4′-trihydroxyflavone singly (FIG. 15).

### <3-3> Confirmation of Anticancer Activity of Buformin and 5,7,4′-trihydroxyflavone Against Lung Cancer

In order to investigate the anticancer activity of buformin and 5,7,4′-trihydroxyflavone, lung cancer cells were treated with buformin and 5,7,4′-trihydroxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 16, it has been confirmed that a ceiling effect showing significantly higher growth inhibition is exerted in a case where lung cancer cells are treated with 10, 100, or 1000 μM buformin and 20 μM 5,7,4′-trihydroxyflavone in combination compared to a case where lung cancer cells are treated with 10, 100, or 1000 μM buformin or 20 μM 5,7,4′-trihydroxyflavone singly (FIG. 16).

### <3-4> Confirmation of Anticancer Activity of Buformin and 5,7,4′-trihydroxyflavone Against Prostate Cancer

In order to investigate the anticancer activity of buformin and 5,7,4′-trihydroxyflavone, prostate cancer cells were treated with buformin and 5,7,4′-trihydroxyflavone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 17, it has been confirmed that a ceiling effect showing significantly higher growth inhibition is exerted in a case where prostate cancer cells are treated with 10, 100, or 1000 μM buformin and 20 μM 5,7,4′-trihydroxyflavone in combination compared to a case where prostate cancer cells are treated with 10, 100, or 1000 μM buformin or 20 μM 5,7,4′-trihydroxyflavone singly (FIG. 17).

### <3-3> Confirmation of Anticancer Activity of Buformin and 5,7,4′-trihydroxyflavone Against Pancreatic Cancer

In order to investigate the anticancer activity of buformin and 5,7,4′-trihydroxyflavone, pancreatic cancer cells were treated with buformin and 5,7,4′-trihydroxyflavone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 18, it has been confirmed that a ceiling effect showing significantly higher growth inhibition is exerted in a case where pancreatic cancer cells are treated with 10, 100, or 1000 μM buformin and 20 μM 5,7,4′-trihydroxyflavone in combination compared to a case where pancreatic cancer cells are treated with 10, 100, or 1000 μM buformin or 20 μM 5,7,4′-trihydroxyflavone singly (FIG. 18).

Through the above results, it has been confirmed that the growth of normal cells is slightly affected but a superior effect of inhibiting the growth of cancer cells is exerted when combined treatment with buformin and 5,7,4′-trihydroxyflavone or a salt thereof is performed.

### <Example 4> Confirmation of Anticancer Activity of Biguanide-Based Compound and Hydroxyflavone Derivative

### <4-1> Confirmation of Anticancer Activity of Biguanide and 5,7,4′-trihydroxyflavone (Apigenin) Against Breast Cancer

In order to investigate the anticancer activity of biguanide and 5,7,4′-trihydroxyflavone, breast cancer cells were treated with biguanide and 5,7,4′-trihydroxyflavone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 19A, it has been confirmed that a ceiling effect showing significantly higher growth inhibition is exerted in a case where breast cancer cells are treated with 10, 100, or 1000 μM biguanide and 20 μM 5,7,4′-trihydroxyflavone in combination compared to a case where breast cancer cells are treated with 10, 100, or 1000 μM biguanide or 20 μM 5,7,4′-trihydroxyflavone singly (FIG. 19A). On the other hand, the growth inhibitory activity is 0% to 27% when human normal epithelial cells are treated with 10, 100, or 1000 μM biguanide and 20 μM 5,7,4′-trihydroxyflavone in combination, and it has been thus confirmed that the combined treatment with biguanide and 5,7,4′-trihydroxyflavone significantly inhibits the growth of cancer cells but slightly affects normal cells (FIG. 19B).

### <4-2> Confirmation of Anticancer Activity of Biguanide and 5,7,4′-trihydroxyflavone Against Colon Cancer

In order to investigate the anticancer activity of biguanide and 5,7,4′-trihydroxyflavone, colon cancer cells were treated with biguanide and 5,7,4′-trihydroxyflavone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 20, it has been confirmed that a ceiling effect showing significantly higher growth inhibition is exerted in a case where colon cancer cells are treated with 10, 100, or 1000 μM biguanide and 20 μM 5,7,4′-trihydroxyflavone in combination compared to a case where colon cancer cells are treated with 10, 100, or 1000 μM biguanide or 20 μM 5,7,4′-trihydroxyflavone singly (FIG. 20).

### <4-3> Confirmation of Anticancer Activity of Biguanide and 5,7,4′-trihydroxyflavone Against Lung Cancer

In order to investigate the anticancer activity of biguanide and 5,7,4′-trihydroxyflavone, lung cancer cells were treated with biguanide and 5,7,4′-trihydroxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 21, it has been confirmed that a ceiling effect showing significantly higher growth inhibition is exerted in a case where lung cancer cells are treated with 10, 100, or 1000 μM biguanide and 20 μM 5,7,4′-trihydroxyflavone in combination compared to a case where lung cancer cells are treated with 10, 100, or 1000 μM biguanide or 20 μM 5,7,4′-trihydroxyflavone singly (FIG. 21).

### <4-4> Confirmation of Anticancer Activity of Biguanide and 5,7,4′-trihydroxyflavone Against Prostate Cancer

In order to investigate the anticancer activity of biguanide and 5,7,4′-trihydroxyflavone, prostate cancer cells were treated with biguanide and 5,7,4′-trihydroxyflavone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 22, it has been confirmed that a ceiling effect showing significantly higher growth inhibition is exerted in a case where prostate cancer cells are treated with 10, 100, or 1000 μM biguanide and 20 μM 5,7,4′-trihydroxyflavone in combination compared to a case where prostate cancer cells are treated with 10, 100, or 1000 μM biguanide or 20 μM 5,7,4′-trihydroxyflavone singly (FIG. 22).

### <4-5> Confirmation of Anticancer Activity of Biguanide and 5,7,4′-Trihydroxyflavone Against Pancreatic Cancer

In order to investigate the anticancer activity of biguanide and 5,7,4′-trihydroxyflavone, pancreatic cancer cells were treated with biguanide and 5,7,4′-trihydroxyflavone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 23, it has been confirmed that a ceiling effect showing significantly higher growth inhibition is exerted in a case where pancreatic cancer cells are treated with 10, 100, or 1000 μM biguanide and 20 μM 5,7,4′-trihydroxyflavone in combination compared to a case where pancreatic cancer cells are treated with 10, 100, or 1000 μM biguanide or 20 μM 5,7,4′-trihydroxyflavone singly (FIG. 23).

Through the above results, it has been confirmed that the growth of normal cells is slightly affected but a superior effect of inhibiting the growth of cancer cells is exerted when combined treatment with biguanide and 5,7,4′-trihydroxyflavone or a salt thereof is performed.

### <Example 5> Confirmation of Anticancer Activity of Biguanide-Based Compound and Hydroxyflavone Derivative

### <5-1> Confirmation of Anticancer Activity of Metformin and 5,7-dihydroxyflavone (Chrysin) Against Breast Cancer

In order to investigate the anticancer activity of metformin and 5,7-dihydroxyflavone, breast cancer cells were treated with metformin and 5,7-dihydroxyflavone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 24A, it has been confirmed that a ceiling effect showing significantly higher growth inhibition is exerted in a case where breast cancer cells are treated with 5 mM metformin and 0.1, 1, or 10 μM 5,7-dihydroxyflavone in combination compared to a case where breast cancer cells are treated with 5 mM metformin or 0.1, 1, or 10 μM 5,7-dihydroxyflavone singly (FIG. 24A). On the other hand, the growth inhibitory activity is 0% to 18% when human normal epithelial cells are treated with 5 mM metformin and 5,7-dihydroxyflavone in combination, and it has been thus confirmed that the combined treatment with metformin and 5,7-dihydroxyflavone significantly inhibits the growth of cancer cells but slightly affects normal cells (FIG. 24B).

### <5-2> Confirmation of Anticancer Activity of Metformin and 5,7-dihydroxyflavone Against Colon Cancer

In order to investigate the anticancer activity of metformin and 5,7-dihydroxyflavone, colon cancer cells were treated with metformin and 5,7-dihydroxyflavone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 25, it has been confirmed that a ceiling effect showing significantly higher growth inhibition is exerted in a case where colon cancer cells are treated with 5 mM metformin and 0.1, 1, or 10 μM 5,7-dihydroxyflavone in combination compared to a case where colon cancer cells are treated with 5 mM metformin or 0.1, 1, or 10 μM 5,7-dihydroxyflavone singly (FIG. 25).

### <5-3> Confirmation of Anticancer Activity of Metformin and 5,7-dihydroxyflavone Against Lung Cancer

In order to investigate the anticancer activity of metformin and 5,7-dihydroxyflavone, lung cancer cells were treated with metformin and 5,7-dihydroxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 26, it has been confirmed that a ceiling effect showing significantly higher growth inhibition is exerted in a case where lung cancer cells are treated with 5 mM metformin and 0.1, 1, or 10 μM 5,7-dihydroxyflavone in combination compared to a case where lung cancer cells are treated with 5 mM metformin or 0.1, 1, or 10 μM 5,7-dihydroxyflavone singly (FIG. 26).

### <5-4> Confirmation of Anticancer Activity of Metformin and 5,7-dihydroxyflavone Against Prostate Cancer

In order to investigate the anticancer activity of metformin and 5,7-dihydroxyflavone, prostate cancer cells were treated with metformin and 5,7-dihydroxyflavone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 27, it has been confirmed that a ceiling effect showing significantly higher growth inhibition is exerted in a case where prostate cancer cells are treated with 5 mM metformin and 0.1, 1, or 10 μM 5,7-dihydroxyflavone in combination compared to a case where prostate cancer cells are treated with 5 mM metformin or 0.1, 1, or 10 μM 5,7-dihydroxyflavone singly (FIG. 27).

### <5-5> Confirmation of Anticancer Activity of Metformin and 5,7-dihydroxyflavone Against Pancreatic Cancer

In order to investigate the anticancer activity of metformin and 5,7-dihydroxyflavone, pancreatic cancer cells were treated with metformin and 5,7-dihydroxyflavone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 28, it has been confirmed that a ceiling effect showing significantly higher growth inhibition is exerted in a case where pancreatic cancer cells are treated with 5 mM metformin and 0.1, 1, or 10 μM 5,7-dihydroxyflavone in combination compared to a case where pancreatic cancer cells are treated with 5 mM metformin or 0.1, 1, or 10 μM 5,7-dihydroxyflavone singly (FIG. 28).

Through the above results, it has been confirmed that the growth of normal cells is slightly affected but a superior effect of inhibiting the growth of cancer cells is exerted when combined treatment with metformin and 5,7-dihydroxyflavone or a salt thereof is performed.

### <Example 6> Confirmation of Anticancer Activity of Biguanide-Based Compound and Hydroxyflavone Derivative

### <6-1> Confirmation of Anticancer Activity of Metformin and 7-O-acetyl Chrysin (Monoacetyl Chrysin) Against Breast Cancer

In order to investigate the anticancer activity of metformin and 7-O-acetyl chrysin, breast cancer cells were treated with metformin and 7-O-acetyl chrysin and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 29A, it has been confirmed that a ceiling effect showing significantly higher growth inhibition is exerted in a case where breast cancer cells are treated with 5 mM metformin and 0.1, 1, or 10 μM 7-O-acetyl chrysin in combination compared to a case where breast cancer cells are treated with 5 mM metformin or 0.1, 1, or 10 μM 7-O-acetyl chrysin singly (FIG. 29A). On the other hand, the growth inhibitory activity is 0% to 18% when human normal epithelial cells are treated with 5 mM metformin and 7-O-acetyl chrysin in combination, and it has been thus confirmed that the combined treatment with metformin and 7-O-acetyl chrysin significantly inhibits the growth of cancer cells but slightly affects normal cells (FIG. 29B).

### <6-2> Confirmation of Anticancer Activity of Metformin and 7-O-acetyl Chrysin Against Colon Cancer

In order to investigate the anticancer activity of metformin and 7-O-acetyl chrysin, colon cancer cells were treated with metformin and 7-O-acetyl chrysin and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 30, it has been confirmed that a ceiling effect showing significantly higher growth inhibition is exerted in a case where colon cancer cells are treated with 5 mM metformin and 0.1, 1, or 10 μM 7-O-acetyl chrysin in combination compared to a case where colon cancer cells are treated with 5 mM metformin or 0.1, 1, or 10 μM 7-O-acetyl chrysin singly (FIG. 30).

### <6-3> Confirmation of Anticancer Activity of Metformin and 7-O-Acetyl Chrysin Against Lung Cancer

In order to investigate the anticancer activity of metformin and 7-O-acetyl chrysin, lung cancer cells were treated with metformin and 7-O-acetyl chrysin and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 31, it has been confirmed that a ceiling effect showing significantly higher growth inhibition is exerted in a case where lung cancer cells are treated with 5 mM metformin and 0.1, 1, or 10 μM 7-O-acetyl chrysin in combination compared to a case where lung cancer cells are treated with 5 mM metformin or 0.1, 1, or 10 μM 7-O-acetyl chrysin singly (FIG. 31).

### <6-4> Confirmation of Anticancer Activity of Metformin and 7-O-Acetyl Chrysin Against Prostate Cancer

In order to investigate the anticancer activity of metformin and 7-O-acetyl chrysin, prostate cancer cells were treated with metformin and 7-O-acetyl chrysin and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 32, it has been confirmed that a ceiling effect showing significantly higher growth inhibition is exerted in a case where prostate cancer cells are treated with 5 mM metformin and 0.1, 1, or 10 μM 7-O-acetyl chrysin in combination compared to a case where prostate cancer cells are treated with 5 mM metformin or 0.1, 1, or 10 μM 7-O-acetyl chrysin singly (FIG. 32).

### <6-5> Confirmation of Anticancer Activity of Metformin and 7-O-acetyl Chrysin Against Pancreatic Cancer

In order to investigate the anticancer activity of metformin and 7-O-acetyl chrysin, pancreatic cancer cells were treated with metformin and 7-O-acetyl chrysin and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 33, it has been confirmed that a ceiling effect showing significantly higher growth inhibition is exerted in a case where pancreatic cancer cells are treated with 5 mM metformin and 0.1, 1, or 10 μM 7-O-acetyl chrysin in combination compared to a case where pancreatic cancer cells are treated with 5 mM metformin or 0.1, 1, or 10 μM 7-O-acetyl chrysin singly (FIG. 33).

Through the above results, it has been confirmed that the growth of normal cells is slightly affected but a superior effect of inhibiting the growth of cancer cells is exerted when combined treatment with metformin and 7-O-acetyl chrysin or a salt thereof is performed.

### <Example 7> Confirmation of Anticancer Activity of Biguanide-Based Compound and Hydroxyflavone Derivative

### <7-1> Confirmation of Anticancer Activity of Metformin and 5,7-di-O-methoxy Chrysin (Dimethyl Chrysin) Against Breast Cancer

In order to investigate the anticancer activity of metformin and 5,7-di-O-methoxy chrysin, breast cancer cells were treated with metformin and 5,7-di-O-methoxy chrysin and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 34A, it has been confirmed that a ceiling effect showing significantly higher growth inhibition is exerted in a case where breast cancer cells are treated with 5 mM metformin and 0.1, 1, or 10 μM 5,7-di-O-methoxy chrysin in combination compared to a case where breast cancer cells are treated with 5 mM metformin or 0.1, 1, or 10 μM 5,7-di-O-methoxy chrysin singly (FIG. 34A). On the other hand, the growth inhibitory activity is 0% to 26% when human normal epithelial cells are treated with 5 mM metformin and 5,7-di-O-methoxy chrysin in combination, and it has been thus confirmed that the combined treatment with metformin and 5,7-di-O-methoxy chrysin significantly inhibits the growth of cancer cells but slightly affects normal cells (FIG. 34B).

### <7-2> Confirmation of Anticancer Activity of Metformin and 5,7-di-O-methoxy Chrysin Against Colon Cancer

In order to investigate the anticancer activity of metformin and 5,7-di-O-methoxy chrysin, colon cancer cells were treated with metformin and 5,7-di-O-methoxy chrysin and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 35, it has been confirmed that a ceiling effect showing significantly higher growth inhibition is exerted in a case where colon cancer cells are treated with 5 mM metformin and 0.1, 1, or 10 μM 5,7-di-O-methoxy chrysin in combination compared to a case where colon cancer cells are treated with 5 mM metformin or 0.1, 1, or 10 μM 5,7-di-O-methoxy chrysin singly (FIG. 35).

### <7-3> Confirmation of Anticancer Activity of Metformin and 5,7-di-O-methoxy Chrysin Against Lung Cancer

In order to investigate the anticancer activity of metformin and 5,7-di-O-methoxy chrysin, lung cancer cells were treated with metformin and 5,7-di-O-methoxy chrysin and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 36, it has been confirmed that a ceiling effect showing significantly higher growth inhibition is exerted in a case where lung cancer cells are treated with 5 mM metformin and 0.1, 1, or 10 μM 5,7-di-O-methoxy chrysin in combination compared to a case where lung cancer cells are treated with 5 mM metformin or 0.1, 1, or 10 μM 5,7-di-O-methoxy chrysin singly (FIG. 36).

### <7-4> Confirmation of Anticancer Activity of Metformin and 5,7-di-O-methoxy Chrysin Against Prostate Cancer

In order to investigate the anticancer activity of metformin and 5,7-di-O-methoxy chrysin, prostate cancer cells were treated with metformin and 5,7-di-O-methoxy chrysin and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 37, it has been confirmed that a ceiling effect showing significantly higher growth inhibition is exerted in a case where prostate cancer cells are treated with 5 mM metformin and 0.1, 1, or 10 μM 5,7-di-O-methoxy chrysin in combination compared to a case where prostate cancer cells are treated with 5 mM metformin or 0.1, 1, or 10 μM 5,7-di-O-methoxy chrysin singly (FIG. 37).

### <7-5> Confirmation of Anticancer Activity of Metformin and 5,7-di-O-methoxy Chrysin Against Pancreatic Cancer

In order to investigate the anticancer activity of metformin and 5,7-di-O-methoxy chrysin, pancreatic cancer cells were treated with metformin and 5,7-di-O-methoxy chrysin and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 38, it has been confirmed that a ceiling effect showing significantly higher growth inhibition is exerted in a case where pancreatic cancer cells are treated with 5 mM metformin and 0.1, 1, or 10 μM 5,7-di-O-methoxy chrysin in combination compared to a case where pancreatic cancer cells are treated with 5 mM metformin or 0.1, 1, or 10 μM 5,7-di-O-methoxy chrysin singly (FIG. 38).

Through the above results, it has been confirmed that the growth of normal cells is slightly affected but a superior effect of inhibiting the growth of cancer cells is exerted when combined treatment with metformin and 5,7-di-O-methoxy chrysin or a salt thereof is performed.

### <Example 8> Confirmation of Anticancer Activity of Biguanide-Based Compound and Hydroxyflavone Derivative

### <8-1> Confirmation of Anticancer Activity of Metformin and 5,7-di-O-acetyl Chrysin (Diacetyl Chrysin) Against Breast Cancer

In order to investigate the anticancer activity of metformin and 5,7-di-O-acetyl chrysin, breast cancer cells were treated with metformin and 5,7-di-O-acetyl chrysin and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 39A, it has been confirmed that a ceiling effect showing significantly higher growth inhibition is exerted in a case where breast cancer cells are treated with 5 mM metformin and 0.1, 1, or 10 μM 5,7-di-O-acetyl chrysin in combination compared to a case where breast cancer cells are treated with 5 mM metformin or 0.1, 1, or 10 μM 5,7-di-O-acetyl chrysin singly (FIG. 39A). On the other hand, the growth inhibitory activity is as weak as 0% to 13% when human normal epithelial cells are treated with 5 mM metformin and 5,7-di-O-acetyl chrysin in combination, and it has been thus confirmed that the combined treatment with metformin and 5,7-di-O-acetyl chrysin significantly inhibits the growth of cancer cells but hardly affects normal cells (FIG. 39B).

### <8-2> Confirmation of Anticancer Activity of Metformin and 5,7-di-O-acetyl Chrysin Against Colon Cancer

In order to investigate the anticancer activity of metformin and 5,7-di-O-acetyl chrysin, colon cancer cells were treated with metformin and 5,7-di-O-acetyl chrysin and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 40, it has been confirmed that a ceiling effect showing significantly higher growth inhibition is exerted in a case where colon cancer cells are treated with 5 mM metformin and 0.1, 1, or 10 μM 5,7-di-O-acetyl chrysin in combination compared to a case where colon cancer cells are treated with 5 mM metformin or 0.1, 1, or 10 μM 5,7-di-O-acetyl chrysin singly (FIG. 40).

### <8-3> Confirmation of Anticancer Activity of Metformin and 5,7-di-O-acetyl Chrysin Against Lung Cancer

In order to investigate the anticancer activity of metformin and 5,7-di-O-acetyl chrysin, lung cancer cells were treated with metformin and 5,7-di-O-acetyl chrysin and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 41, it has been confirmed that a ceiling effect showing significantly higher growth inhibition is exerted in a case where lung cancer cells are treated with 5 mM metformin and 0.1, 1, or 10 μM 5,7-di-O-acetyl chrysin in combination compared to a case where lung cancer cells are treated with 5 mM metformin or 0.1, 1, or 10 μM 5,7-di-O-acetyl chrysin singly (FIG. 41).

### <8-4> Confirmation of Anticancer Activity of Metformin and 5,7-di-O-acetyl Chrysin Against Prostate Cancer

In order to investigate the anticancer activity of metformin and 5,7-di-O-acetyl chrysin, prostate cancer cells were treated with metformin and 5,7-di-O-acetyl chrysin and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 42, it has been confirmed that a ceiling effect showing significantly higher growth inhibition is exerted in a case where prostate cancer cells are treated with 5 mM metformin and 0.1, 1, or 10 μM 5,7-di-O-acetyl chrysin in combination compared to a case where prostate cancer cells are treated with 5 mM metformin or 0.1, 1, or 10 μM 5,7-di acetyl chrysin singly (FIG. 42).

### <8-5> Confirmation of Anticancer Activity of Metformin and 5,7-di-O-acetyl Chrysin Against Pancreatic Cancer

In order to investigate the anticancer activity of metformin and 5,7-di-O-acetyl chrysin, pancreatic cancer cells were treated with metformin and 5,7-di-O-acetyl chrysin and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 43, it has been confirmed that a ceiling effect showing significantly higher growth inhibition is exerted in a case where pancreatic cancer cells are treated with 5 mM metformin and 0.1, 1, or 10 μM 5,7-di-O-acetyl chrysin in combination compared to a case where pancreatic cancer cells are treated with 5 mM metformin or 0.1, 1, or 10 μM 5,7-di-O-acetyl chrysin singly (FIG. 43).

Through the above results, it has been confirmed that the growth of normal cells is slightly affected but a superior effect of inhibiting the growth of cancer cells is exerted when combined treatment with metformin and 5,7-di-O-acetyl chrysin or a salt thereof is performed.

### <Example 9> Confirmation of Anticancer Activity of Biguanide-Based Compound and Hydroxyflavone Derivative

### <9-1> Confirmation of Anticancer Activity of Metformin and 3′,4′,5,7-tetrahydroxyflavone (Luteolin) Against Breast Cancer

In order to investigate the anticancer activity of metformin and 3′,4′,5,7-tetrahydroxyflavone, breast cancer cells were treated with metformin and 3′,4′,5,7-tetrahydroxyflavone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 44A, it has been confirmed that a ceiling effect showing significantly higher growth inhibition is exerted in a case where breast cancer cells are treated with 5 mM metformin and 0.1, 1, or 10 μM 3′,4′,5,7-tetrahydroxyflavone in combination compared to a case where breast cancer cells are treated with 5 mM metformin or 0.1, 1, or 10 μM 3′,4′,5,7-tetrahydroxyflavone singly (FIG. 44A). On the other hand, the growth inhibitory activity is as weak as 0% to 9% when human normal epithelial cells are treated with 5 mM metformin and 3′,4′,5,7-tetrahydroxyflavone in combination, and it has been thus confirmed that the combined treatment with metformin and 3′,4′,5,7-tetrahydroxyflavone significantly inhibits the growth of cancer cells but hardly affects normal cells (FIG. 44B).

### <9-2> Confirmation of Anticancer Activity of Metformin and 3′,4′,5,7-tetrahydroxyflavone Against Colon Cancer

In order to investigate the anticancer activity of metformin and 3′,4′,5,7-tetrahydroxyflavone, colon cancer cells were treated with metformin and 3′,4′,5,7-tetrahydroxyflavone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 45, it has been confirmed that a ceiling effect showing significantly higher growth inhibition is exerted in a case where colon cancer cells are treated with 5 mM metformin and 0.1, 1, or 10 μM 3′,4′,5,7-tetrahydroxyflavone in combination compared to a case where colon cancer cells are treated with 5 mM metformin or 0.1, 1, or 10 μM 3′,4′,5,7-tetrahydroxyflavone singly (FIG. 45).

### <9-3> Confirmation of Anticancer Activity of Metformin and 3′,4′,5,7-tetrahydroxyflavone Against Lung Cancer

In order to investigate the anticancer activity of metformin and 3′,4′,5,7-tetrahydroxyflavone, lung cancer cells were treated with metformin and 3′,4′,5,7-tetrahydroxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 46, it has been confirmed that a ceiling effect showing significantly higher growth inhibition is exerted in a case where lung cancer cells are treated with 5 mM metformin and 0.1, 1, or 10 μM 3′,4′,5,7-tetrahydroxyflavone in combination compared to a case where lung cancer cells are treated with 5 mM metformin or 0.1, 1, or 10 μM 3′,4′,5,7-tetrahydroxyflavone singly (FIG. 46).

### <9-4> Confirmation of Anticancer Activity of Metformin and 3′,4′,5,7-tetrahydroxyflavone Against Prostate Cancer

In order to investigate the anticancer activity of metformin and 3′,4′,5,7-tetrahydroxyflavone, prostate cancer cells were treated with metformin and 3′,4′,5,7-tetrahydroxyflavone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 47, it has been confirmed that a ceiling effect showing significantly higher growth inhibition is exerted in a case where prostate cancer cells are treated with 5 mM metformin and 0.1, 1, or 10 μM 3′,4′,5,7-tetrahydroxyflavone in combination compared to a case where prostate cancer cells are treated with 5 mM metformin or 0.1, 1, or 10 μM 3′,4′,5,7-tetrahydroxyflavone singly (FIG. 47).

### <9-5> Confirmation of Anticancer Activity of Metformin and 3′,4′,5,7-tetrahydroxyflavone Against Pancreatic Cancer

In order to investigate the anticancer activity of metformin and 3′,4′,5,7-tetrahydroxyflavone, pancreatic cancer cells were treated with metformin and 3′,4′,5,7-tetrahydroxyflavone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 48, it has been confirmed that a ceiling effect showing significantly higher growth inhibition is exerted in a case where pancreatic cancer cells are treated with 5 mM metformin and 0.1, 1, or 10 μM 3′,4′,5,7-tetrahydroxyflavone in combination compared to a case where pancreatic cancer cells are treated with 5 mM metformin or 0.1, 1, or 10 μM 3′,4′,5,7-tetrahydroxyflavone singly (FIG. 48).

Through the above results, it has been confirmed that the growth of normal cells is slightly affected but a superior effect of inhibiting the growth of cancer cells is exerted when combined treatment with metformin and 3′,4′,5,7-tetrahydroxyflavone or a salt thereof is performed.

### <Example 10> Confirmation of Anticancer Activity of Biguanide-Based Compound and Hydroxyflavone Derivative

### <10-1> Confirmation of Anticancer Activity of Metformin and 3,4′,5,7-tetrahydroxyflavone (Kaempferol) Against Breast Cancer

In order to investigate the anticancer activity of metformin and 3,4′,5,7-tetrahydroxyflavone, breast cancer cells were treated with metformin and 3,4′,5,7-tetrahydroxyflavone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 49A, it has been confirmed that a ceiling effect showing significantly higher growth inhibition is exerted in a case where breast cancer cells are treated with 5 mM metformin and 0.1, 1, or 10 μM 3,4′,5,7-tetrahydroxyflavone in combination compared to a case where breast cancer cells are treated with 5 mM metformin or 0.1, 1, or 10 μM 3,4′,5,7-tetrahydroxyflavone singly (FIG. 49A). On the other hand, the growth inhibitory activity is as weak as 0% to 12% when human normal epithelial cells are treated with 5 mM metformin and 3,4′,5,7-tetrahydroxyflavone in combination, and it has been thus confirmed that the combined treatment with metformin and 3,4′,5,7-tetrahydroxyflavone significantly inhibits the growth of cancer cells but hardly affects normal cells (FIG. 49B).

### <10-2> Confirmation of Anticancer Activity of Metformin and 3,4′,5,7-tetrahydroxyflavone Against Colon Cancer

In order to investigate the anticancer activity of metformin and 3,4′,5,7-tetrahydroxyflavone, colon cancer cells were treated with metformin and 3,4′,5,7-tetrahydroxyflavone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 50, it has been confirmed that a ceiling effect showing significantly higher growth inhibition is exerted in a case where colon cancer cells are treated with 5 mM metformin and 0.1, 1, or 10 μM 3,4′,5,7-tetrahydroxyflavone in combination compared to a case where colon cancer cells are treated with 5 mM metformin or 0.1, 1, or 10 μM 3,4′,5,7-tetrahydroxyflavone singly (FIG. 50).

### <10-3> Confirmation of Anticancer Activity of Metformin and 3,4′,5,7-tetrahydroxyflavone Against Lung Cancer

In order to investigate the anticancer activity of metformin and 3,4′,5,7-tetrahydroxyflavone, lung cancer cells were treated with metformin and 3,4′,5,7-tetrahydroxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 51, it has been confirmed that a ceiling effect showing significantly higher growth inhibition is exerted in a case where lung cancer cells are treated with 5 mM metformin and 0.1, 1, or 10 μM 3,4′,5,7-tetrahydroxyflavone in combination compared to a case where lung cancer cells are treated with 5 mM metformin or 0.1, 1, or 10 μM 3,4′,5,7-tetrahydroxyflavone singly (FIG. 51).

### <10-4> Confirmation of Anticancer Activity of Metformin and 3,4′,5,7-tetrahydroxyflavone Against Prostate Cancer

In order to investigate the anticancer activity of metformin and 3,4′,5,7-tetrahydroxyflavone, prostate cancer cells were treated with metformin and 3,4′,5,7-tetrahydroxyflavone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 52, it has been confirmed that a ceiling effect showing significantly higher growth inhibition is exerted in a case where prostate cancer cells are treated with 5 mM metformin and 0.1, 1, or 10 μM 3,4′,5,7-tetrahydroxyflavone in combination compared to a case where prostate cancer cells are treated with 5 mM metformin or 0.1, 1, or 10 μM 3,4′,5,7-tetrahydroxyflavone singly (FIG. 52).

### <10-5> Confirmation of Anticancer Activity of Metformin and 3,4′,5,7-tetrahydroxyflavone Against Pancreatic Cancer

In order to investigate the anticancer activity of metformin and 3,4′,5,7-tetrahydroxyflavone, pancreatic cancer cells were treated with metformin and 3,4′,5,7-tetrahydroxyflavone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 53, it has been confirmed that a ceiling effect showing significantly higher growth inhibition is exerted in a case where pancreatic cancer cells are treated with 5 mM metformin and 0.1, 1, or 10 μM 3,4′,5,7-tetrahydroxyflavone in combination compared to a case where pancreatic cancer cells are treated with 5 mM metformin or 0.1, 1, or 10 μM 3,4′,5,7-tetrahydroxyflavone singly (FIG. 53).

Through the above results, it has been confirmed that the growth of normal cells is slightly affected but a superior effect of inhibiting the growth of cancer cells is exerted when combined treatment with metformin and 3,4′,5,7-tetrahydroxyflavone or a salt thereof is performed.

### <Example 11> Confirmation of Anticancer Activity of Biguanide-Based Compound and Hydroxyflavone Derivative

### <11-1> Confirmation of Anticancer Activity of Metformin and 5,7,3′,4′-flavon-3-ol (Quercetin) Against Breast Cancer

In order to investigate the anticancer activity of metformin and 5,7,3′,4′-flavon-3-ol, breast cancer cells were treated with metformin and 5,7,3′,4′-flavon-3-ol and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 54A, it has been confirmed that a ceiling effect showing significantly higher growth inhibition is exerted in a case where breast cancer cells are treated with 5 mM metformin and 0.1, 1, or 10 μM 5,7,3′,4′-flavon-3-ol in combination compared to a case where breast cancer cells are treated with 5 mM metformin or 0.1, 1, or 10 μM 5,7,3′,4′-flavon-3-ol singly (FIG. 54A). On the other hand, the growth inhibitory activity is as weak as 0% to 16% when human normal epithelial cells are treated with 5 mM metformin and 5,7,3′,4′-flavon-3-ol in combination, and it has been thus confirmed that the combined treatment with metformin and 5,7,3′,4′-flavon-3-ol significantly inhibits the growth of cancer cells but hardly affects normal cells (FIG. 54B).

### <11-2> Confirmation of Anticancer Activity of Metformin and 5,7,3′,4′-flavon-3-ol Against Colon Cancer

In order to investigate the anticancer activity of metformin and 5,7,3′,4′-flavon-3-ol, colon cancer cells were treated with metformin and 5,7,3′,4′-flavon-3-ol and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 55, it has been confirmed that a ceiling effect showing significantly higher growth inhibition is exerted in a case where colon cancer cells are treated with 5 mM metformin and 0.1, 1, or 10 μM 5,7,3′,4′-flavon-3-ol in combination compared to a case where colon cancer cells are treated with 5 mM metformin or 0.1, 1, or 10 μM 5,7,3′,4′-flavon-3-ol singly (FIG. 55).

### <11-3> Confirmation of Anticancer Activity of Metformin and 5,7,3′,4′-flavon-3-ol Against Lung Cancer

In order to investigate the anticancer activity of metformin and 5,7,3′,4′-flavon-3-ol, lung cancer cells were treated with metformin and 5,7,3′,4′-flavon-3-ol and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 56, it has been confirmed that a ceiling effect showing significantly higher growth inhibition is exerted in a case where lung cancer cells are treated with 5 mM metformin and 0.1, 1, or 10 μM 5,7,3′,4′-flavon-3-ol in combination compared to a case where lung cancer cells are treated with 5 mM metformin or 0.1, 1, or 10 μM 5,7,3′,4′-flavon-3-ol singly (FIG. 56).

### <11-4> Confirmation of Anticancer Activity of Metformin and 5,7,3′,4′-flavon-3-ol Against Prostate Cancer

In order to investigate the anticancer activity of metformin and 5,7,3′,4′-flavon-3-ol, prostate cancer cells were treated with metformin and 5,7,3′,4′-flavon-3-ol and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 57, it has been confirmed that a ceiling effect showing significantly higher growth inhibition is exerted in a case where prostate cancer cells are treated with 5 mM metformin and 0.1, 1, or 10 μM 5,7,3′,4′-flavon-3-ol in combination compared to a case where prostate cancer cells are treated with 5 mM metformin or 0.1, 1, or 10 μM 5,7,3′,4′-flavon-3-ol singly (FIG. 57).

### <11-5> Confirmation of Anticancer Activity of Metformin and 5,7,3′,4′-flavon-3-ol Against Pancreatic Cancer

In order to investigate the anticancer activity of metformin and 5,7,3′,4′-flavon-3-ol, pancreatic cancer cells were treated with metformin and 5,7,3′,4′-flavon-3-ol and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 58, it has been confirmed that a ceiling effect showing significantly higher growth inhibition is exerted in a case where pancreatic cancer cells are treated with 5 mM metformin and 0.1, 1, or 10 μM 5,7,3′,4′-flavon-3-ol in combination compared to a case where pancreatic cancer cells are treated with 5 mM metformin or 0.1, 1, or 10 μM 5,7,3′,4′-flavon-3-ol singly (FIG. 58).

Through the above results, it has been confirmed that the growth of normal cells is slightly affected but a superior effect of inhibiting the growth of cancer cells is exerted when combined treatment with metformin and 5,7,3′,4′-flavon-3-ol or a salt thereof is performed.

### <Example 12> Confirmation of Anticancer Activity of Biguanide-Based Compound and Hydroxyflavone Derivative

### <12-1> Confirmation of Anticancer Activity of Metformin and 7,3′,4′-flavon-3-ol (Fisetin) Against Breast Cancer

In order to investigate the anticancer activity of metformin and 7,3′,4′-flavon-3-ol, breast cancer cells were treated with metformin and 7,3′,4′-flavon-3-ol and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 59A, it has been confirmed that a ceiling effect showing significantly higher growth inhibition is exerted in a case where breast cancer cells are treated with 5 mM metformin and 0.1, 1, or 10 μM 7,3′,4′-flavon-3-ol in combination compared to a case where breast cancer cells are treated with 5 mM metformin or 0.1, 1, or 10 μM 7,3′,4′-flavon-3-ol singly (FIG. 59A). On the other hand, the growth inhibitory activity is as weak as 0% to 15% when human normal epithelial cells are treated with 5 mM metformin and 7,3′,4′-flavon-3-ol in combination, and it has been thus confirmed that the combined treatment with metformin and 7,3′,4′-flavon-3-ol significantly inhibits the growth of cancer cells but hardly affects normal cells (FIG. 59B).

### <12-2> Confirmation of Anticancer Activity of Metformin and 7,3′,4′-flavon-3-ol Against Colon Cancer

In order to investigate the anticancer activity of metformin and 7,3′,4′-flavon-3-ol, colon cancer cells were treated with metformin and 7,3′,4′-flavon-3-ol and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 60, it has been confirmed that a ceiling effect showing significantly higher growth inhibition is exerted in a case where colon cancer cells are treated with 5 mM metformin and 0.1, 1, or 10 μM 7,3′,4′-flavon-3-ol in combination compared to a case where colon cancer cells are treated with 5 mM metformin or 0.1, 1, or 10 μM 7,3′,4′-flavon-3-ol singly (FIG. 60).

### <12-3> Confirmation of Anticancer Activity of Metformin and 7,3′,4′-flavon-3-ol Against Lung Cancer

In order to investigate the anticancer activity of metformin and 7,3′,4′-flavon-3-ol, lung cancer cells were treated with metformin and 7,3′,4′-flavon-3-ol and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 61, it has been confirmed that a ceiling effect showing significantly higher growth inhibition is exerted in a case where lung cancer cells are treated with 5 mM metformin and 0.1, 1, or 10 μM 7,3′,4′-flavon-3-ol in combination compared to a case where lung cancer cells are treated with 5 mM metformin or 0.1, 1, or 10 μM 7,3′,4′-flavon-3-ol singly (FIG. 61).

### <12-4> Confirmation of Anticancer Activity of Metformin and 7,3′,4′-flavon-3-ol Against Prostate Cancer

In order to investigate the anticancer activity of metformin and 7,3′,4′-flavon-3-ol, prostate cancer cells were treated with metformin and 7,3′,4′-flavon-3-ol and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 62, it has been confirmed that a ceiling effect showing significantly higher growth inhibition is exerted in a case where prostate cancer cells are treated with 5 mM metformin and 0.1, 1, or 10 μM 7,3′,4′-flavon-3-ol in combination compared to a case where prostate cancer cells are treated with 5 mM metformin or 0.1, 1, or 10 μM 7,3′,4′-flavon-3-ol singly (FIG. 62).

### <12-5> Confirmation of Anticancer Activity of Metformin and 7,3′,4′-flavon-3-ol Against Pancreatic Cancer

In order to investigate the anticancer activity of metformin and 7,3′,4′-flavon-3-ol, pancreatic cancer cells were treated with metformin and 7,3′,4′-flavon-3-ol and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 63, it has been confirmed that a ceiling effect showing significantly higher growth inhibition is exerted in a case where pancreatic cancer cells are treated with 5 mM metformin and 0.1, 1, or 10 μM 7,3′,4′-flavon-3-ol in combination compared to a case where pancreatic cancer cells are treated with 5 mM metformin or 0.1, 1, or 10 μM 7,3′,4′-flavon-3-ol singly (FIG. 63).

Through the above results, it has been confirmed that the growth of normal cells is slightly affected but a superior effect of inhibiting the growth of cancer cells is exerted when combined treatment with metformin and 7,3′,4′-flavon-3-ol or a salt thereof is performed.

### <Example 13> Confirmation of Anticancer Activity of Biguanide-Based Compound and Hydroxyflavone Derivative

### <13-1> Confirmation of Anticancer Activity of Metformin and 4′,5-dihydroxy-7-methoxyflavone (Genkwanin) Against Breast Cancer

In order to investigate the anticancer activity of metformin and 4′,5-dihydroxy-7-methoxyflavone, breast cancer cells were treated with metformin and 4′,5-dihydroxy-7-methoxyflavone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 64A, it has been confirmed that a ceiling effect showing significantly higher growth inhibition is exerted in a case where breast cancer cells are treated with 5 mM metformin and 0.1, 1, or 10 μM 4′,5-dihydroxy-7-methoxyflavone in combination compared to a case where breast cancer cells are treated with 5 mM metformin or 0.1, 1, or 10 μM 4′,5-dihydroxy-7-methoxyflavone singly (FIG. 64A). On the other hand, the growth inhibitory activity is 0% to 33% when human normal epithelial cells are treated with 5 mM metformin and 4′,5-dihydroxy-7-methoxyflavone in combination, and it has been thus confirmed that the combined treatment with metformin and 4′,5-dihydroxy methoxyflavone significantly inhibits the growth of cancer cells but slightly affects normal cells (FIG. 64B).

### <13-2> Confirmation of Anticancer Activity of Metformin and 4′,5-dihydroxy-7-methoxyflavone Against Colon Cancer

In order to investigate the anticancer activity of metformin and 4′,5-dihydroxy-7-methoxyflavone, colon cancer cells were treated with metformin and 4′,5-dihydroxy-7-methoxyflavone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 65, it has been confirmed that a ceiling effect showing significantly higher growth inhibition is exerted in a case where colon cancer cells are treated with 5 mM metformin and 0.1, 1, or 10 μM 4′,5-dihydroxy-7-methoxyflavone in combination compared to a case where colon cancer cells are treated with 5 mM metformin or 0.1, 1, or 10 μM 4′,5-dihydroxy-7-methoxyflavone singly (FIG. 65).

### <13-3> Confirmation of Anticancer Activity of Metformin and 4′,5-dihydroxy-7-methoxyflavone Against Lung Cancer

In order to investigate the anticancer activity of metformin and 4′,5-dihydroxy-7-methoxyflavone, lung cancer cells were treated with metformin and 4′,5-dihydroxy-7-methoxyflavone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 66, it has been confirmed that a ceiling effect showing significantly higher growth inhibition is exerted in a case where lung cancer cells are treated with 5 mM metformin and 0.1, 1, or 10 μM 4′,5-dihydroxy-7-methoxyflavone in combination compared to a case where lung cancer cells are treated with 5 mM metformin or 0.1, 1, or 10 μM 4′,5-dihydroxy-7-methoxyflavone singly (FIG. 66).

### <13-4> Confirmation of Anticancer Activity of Metformin and 4′,5-dihydroxy-7-methoxyflavone Against Prostate Cancer

In order to investigate the anticancer activity of metformin and 4′,5-dihydroxy-7-methoxyflavone, prostate cancer cells were treated with metformin and 4′,5-dihydroxy-7-methoxyflavone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 67, it has been confirmed that a ceiling effect showing significantly higher growth inhibition is exerted in a case where prostate cancer cells are treated with 5 mM metformin and 0.1, 1, or 10 μM 4′,5-dihydroxy-7-methoxyflavone in combination compared to a case where prostate cancer cells are treated with 5 mM metformin or 0.1, 1, or 10 μM 4′,5-dihydroxy-7-methoxyflavone singly (FIG. 67).

### <13-5> Confirmation of Anticancer Activity of Metformin and 4′,5-dihydroxy-7-methoxyflavone Against Pancreatic Cancer

In order to investigate the anticancer activity of metformin and 4′,5-dihydroxy-7-methoxyflavone, pancreatic cancer cells were treated with metformin and 4′,5-dihydroxy-7-methoxyflavone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 68, it has been confirmed that a ceiling effect showing significantly higher growth inhibition is exerted in a case where pancreatic cancer cells are treated with 5 mM metformin and 0.1, 1, or 10 μM 4′,5-dihydroxy-7-methoxyflavone in combination compared to a case where pancreatic cancer cells are treated with 5 mM metformin or 0.1, 1, or 10 μM 4′,5-dihydroxy-7-methoxyflavone singly (FIG. 68).

Through the above results, it has been confirmed that the growth of normal cells is slightly affected but a superior effect of inhibiting the growth of cancer cells is exerted when combined treatment with metformin and 4′,5-dihydroxy-7-methoxyflavone or a salt thereof is performed.

### <Example 14> Confirmation of Anticancer Activity of Biguanide-Based Compound and Flavanone Derivative

### <14-1> Confirmation of Anticancer Activity of Metformin and 4′,5,7-trihydroxyflavanone (Naringenin) Against Breast Cancer

In order to investigate the anticancer activity of metformin and 4′,5,7-trihydroxyflavanone, breast cancer cells were treated with metformin and 4′,5,7-trihydroxyflavanone and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 69A, it has been confirmed that a ceiling effect showing significantly higher growth inhibition is exerted in a case where breast cancer cells are treated with 5 mM metformin and 0.1, 1, or 10 μM 4′,5,7-trihydroxyflavanone in combination compared to a case where breast cancer cells are treated with 5 mM metformin or 0.1, 1, or 10 μM 4′,5,7-trihydroxyflavanone singly (FIG. 69A). On the other hand, the growth inhibitory activity is as weak as 0% to 4% when human normal epithelial cells are treated with 5 mM metformin and 4′,5,7-trihydroxyflavanone in combination, and it has been thus confirmed that the combined treatment with metformin and 4′,5,7-trihydroxyflavanone significantly inhibits the growth of cancer cells but hardly affects normal cells (FIG. 69B).

### <14-2> Confirmation of Anticancer Activity of Metformin and 4′,5,7-trihydroxyflavanone Against Colon Cancer

In order to investigate the anticancer activity of metformin and 4′,5,7-trihydroxyflavanone, colon cancer cells were treated with metformin and 4′,5,7-trihydroxyflavanone and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 70, it has been confirmed that a ceiling effect showing significantly higher growth inhibition is exerted in a case where colon cancer cells are treated with 5 mM metformin and 0.1, 1, or 10 μM 4′,5,7-trihydroxyflavanone in combination compared to a case where colon cancer cells are treated with 5 mM metformin or 0.1, 1, or 10 μM 4′,5,7-trihydroxyflavanone singly (FIG. 70).

### <14-3> Confirmation of Anticancer Activity of Metformin and 4′,5,7-trihydroxyflavanone Against Lung Cancer

In order to investigate the anticancer activity of metformin and 4′,5,7-trihydroxyflavanone, lung cancer cells were treated with metformin and 4′,5,7-trihydroxyflavanone and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 71, it has been confirmed that a ceiling effect showing significantly higher growth inhibition is exerted in a case where lung cancer cells are treated with 5 mM metformin and 0.1, 1, or 10 μM 4′,5,7-trihydroxyflavanone in combination compared to a case where lung cancer cells are treated with 5 mM metformin or 0.1, 1, or 10 μM 4′,5,7-trihydroxyflavanone singly (FIG. 71).

### <14-4> Confirmation of Anticancer Activity of Metformin and 4′,5,7-trihydroxyflavanone Against Prostate Cancer

In order to investigate the anticancer activity of metformin and 4′,5,7-trihydroxyflavanone, prostate cancer cells were treated with metformin and 4′,5,7-trihydroxyflavanone and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 72, it has been confirmed that a ceiling effect showing significantly higher growth inhibition is exerted in a case where prostate cancer cells are treated with 5 mM metformin and 0.1, 1, or 10 μM 4′,5,7-trihydroxyflavanone in combination compared to a case where prostate cancer cells are treated with 5 mM metformin or 0.1, 1, or 10 μM 4′,5,7-trihydroxyflavanone singly (FIG. 72).

### <14-5> Confirmation of Anticancer Activity of Metformin and 4′,5,7-trihydroxyflavanone Against Pancreatic Cancer

In order to investigate the anticancer activity of metformin and 4′,5,7-trihydroxyflavanone, pancreatic cancer cells were treated with metformin and 4′,5,7-trihydroxyflavanone and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 73, it has been confirmed that a ceiling effect showing significantly higher growth inhibition is exerted in a case where pancreatic cancer cells are treated with 5 mM metformin and 0.1, 1, or 10 μM 4′,5,7-trihydroxyflavanone in combination compared to a case where pancreatic cancer cells are treated with 5 mM metformin or 0.1, 1, or 10 μM 4′,5,7-trihydroxyflavanone singly (FIG. 73).

Through the above results, it has been confirmed that the growth of normal cells is slightly affected but a superior effect of inhibiting the growth of cancer cells is exerted when combined treatment with metformin and 4′,5,7-trihydroxyflavanone or a salt thereof is performed.

### <Example 15> Confirmation of Anticancer Activity of Biguanide-Based Compound and Flavanone Derivative

### <15-1> Confirmation of Anticancer Activity of Metformin and 4′,5,7-trihydroxyflavanone-7-rhamnoglucoside (Naringin) Against Breast Cancer

In order to investigate the anticancer activity of metformin and 4′,5,7-trihydroxyflavanone-7-rhamnoglucoside, breast cancer cells were treated with metformin and 4′,5,7-trihydroxyflavanone rhamnoglucoside and MTT analysis was conducted to examine growth inhibition.

Specifically, MTT analysis was conducted in the same manner as that described in Example <1-1> using the MDA-MB-231 cell line, which was a breast cancer cell line. At this time, the cell line was treated with drugs and incubated for 72 hours. For the normal cell control group, MTT analysis was conducted in the same manner as that described in Example <1-1> using the WISH (human normal epithelial cells) cell line.

As a result, as illustrated in FIG. 74A, it has been confirmed that a ceiling effect showing significantly higher growth inhibition is exerted in a case where breast cancer cells are treated with 5 mM metformin and 0.1, 1, or 10 μM 4′,5,7-trihydroxyflavanone-7-rhamnoglucoside in combination compared to a case where breast cancer cells are treated with 5 mM metformin or 0.1, 1, or 10 μM 4′,5,7-trihydroxyflavanone-7-rhamnoglucoside singly (FIG. 74A). On the other hand, the growth inhibitory activity is as weak as 0% to 9% when human normal epithelial cells are treated with 5 mM metformin and 4′,5,7-trihydroxyflavanone-7-rhamnoglucoside in combination, and it has been thus confirmed that the combined treatment with metformin and 4′,5,7-trihydroxyflavanone-7-rhamnoglucoside significantly inhibits the growth of cancer cells but hardly affects normal cells (FIG. 74B).

### <15-2> Confirmation of Anticancer Activity of Metformin and 4′,5,7-trihydroxyflavanone-7-rhamnoglucoside Against Colon Cancer

In order to investigate the anticancer activity of metformin and 4′,5,7-trihydroxyflavanone-7-rhamnoglucoside, colon cancer cells were treated with metformin and 4′,5,7-trihydroxyflavanone rhamnoglucoside and MTT analysis was conducted to examine growth inhibition.

Specifically, MTT analysis was conducted in the same manner as that described in Example <1-1> using the HCT 116 cell line, which was a colon cancer cell line. At this time, the cell line was treated with drugs and incubated for 72 hours.

As a result, as illustrated in FIG. 75, it has been confirmed that a ceiling effect showing significantly higher growth inhibition is exerted in a case where colon cancer cells are treated with 5 mM metformin and 0.1, 1, or 10 μM 4′,5,7-trihydroxyflavanone-7-rhamnoglucoside in combination compared to a case where colon cancer cells are treated with 5 mM metformin or 0.1, 1, or 10 μM 4′,5,7-trihydroxyflavanone-7-rhamnoglucoside singly (FIG. 75).

### <15-3> Confirmation of Anticancer Activity of Metformin and 4′,5,7-trihydroxyflavanone-7-rhamnoglucoside Against Lung Cancer

In order to investigate the anticancer activity of metformin and 4′,5,7-trihydroxyflavanone-7-rhamnoglucoside, lung cancer cells were treated with metformin and 4′,5,7-trihydroxyflavanone-7-rhamnoglucoside and MTT analysis was conducted to examine growth inhibition.

Specifically, MTT analysis was conducted in the same manner as that described in Example <1-1> using the HCC1195 cell line, which was a lung cancer cell line. At this time, the cell line was treated with drugs and incubated for 72 hours.

As a result, as illustrated in FIG. 76, it has been confirmed that a ceiling effect showing significantly higher growth inhibition is exerted in a case where lung cancer cells are treated with 5 mM metformin and 0.1, 1, or 10 μM 4′,5,7-trihydroxyflavanone-7-rhamnoglucoside in combination compared to a case where lung cancer cells are treated with 5 mM metformin or 0.1, 1, or 10 μM 4′,5,7-trihydroxyflavanone-7-rhamnoglucoside singly (FIG. 76).

### <15-4> Confirmation of Anticancer Activity of Metformin and 4′,5,7-trihydroxyflavanone-7-rhamnoglucoside Against Prostate Cancer

In order to investigate the anticancer activity of metformin and 4′,5,7-trihydroxyflavanone-7-rhamnoglucoside, prostate cancer cells were treated with metformin and 4′,5,7-trihydroxyflavanone-7-rhamnoglucoside and MTT analysis was conducted to examine growth inhibition.

Specifically, MTT analysis was conducted in the same manner as that described in Example <1-1> using the DU145 cell line, which was a prostate cancer cell line. At this time, the cell line was treated with drugs and incubated for 72 hours.

As a result, as illustrated in FIG. 77, it has been confirmed that a ceiling effect showing significantly higher growth inhibition is exerted in a case where prostate cancer cells are treated with 5 mM metformin and 0.1, 1, or 10 μM 4′,5,7-trihydroxyflavanone-7-rhamnoglucoside in combination compared to a case where prostate cancer cells are treated with 5 mM metformin or 0.1, 1, or 10 μM 4′,5,7-trihydroxyflavanone-7-rhamnoglucoside singly (FIG. 77).

### <15-5> Confirmation of Anticancer Activity of Metformin and 4′,5,7-trihydroxyflavanone Rhamnoglucoside Against Pancreatic Cancer

In order to investigate the anticancer activity of metformin and 4′,5,7-trihydroxyflavanone rhamnoglucoside, pancreatic cancer cells were treated with metformin and 4′,5,7-trihydroxyflavanone rhamnoglucoside and MTT analysis was conducted to examine growth inhibition.

Specifically, MTT analysis was conducted in the same manner as that described in Example <1-1> using the AsPC-1 cell line, which was a pancreatic cancer cell line. At this time, the cell line was treated with drugs and incubated for 72 hours.

As a result, as illustrated in FIG. 78, it has been confirmed that a ceiling effect showing significantly higher growth inhibition is exerted in a case where pancreatic cancer cells are treated with 5 mM metformin and 0.1, 1, or 10 μM 4′,5,7-trihydroxyflavanone-7-rhamnoglucoside in combination compared to a case where pancreatic cancer cells are treated with 5 mM metformin or 0.1, 1, or 10 μM 4′,5,7-trihydroxyflavanone-7-rhamnoglucoside singly (FIG. 78).

Through the above results, it has been confirmed that the growth of normal cells is slightly affected but a superior effect of inhibiting the growth of cancer cells is exerted when combined treatment with metformin and 4′,5,7-trihydroxyflavanone-7-rhamnoglucoside or a salt thereof is performed.

In conclusion, when combined treatment with a biguanide-based compound and a flavone derivative, hydroxyflavone derivative, or flavanone derivative is performed, the growth of normal cells is slightly affected but an excellent effect of inhibiting the growth of cancer cells is exerted in various kinds of cancer cells.

1. A method for treatment of cancer, the method comprising administering to an individual in a mixed or combined manner a pharmaceutical composition comprising pharmaceutically effective amounts of
a first component including a biguanide-based compound or a pharmaceutically acceptable salt thereof; and
a second component including a flavone, a hydroxyflavone, a flavanone, a flavone derivative, a hydroxyflavone derivative, a flavanone derivative or a pharmaceutically acceptable salt thereof.

2. A method for treatment of cancer, the method comprising administering to an individual in a mixed or combined manner a pharmaceutical composition comprising pharmaceutically effective amounts of
a first component including a biguanide-based compound or a pharmaceutically acceptable salt thereof; and
a second component including a compound represented by Chemical Formula 5 or a pharmaceutically acceptable salt thereof: 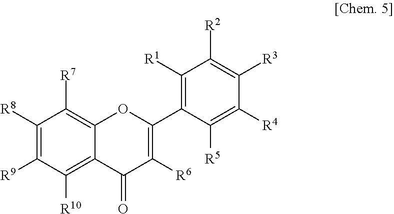
where R¹ to R⁵ are each independently —H, —OH, CₖH₂ₖ₊₁O— or CₖH₂ₖ₊₁COO— (k is an integer from 1 to 5),
R⁶ is —H, —OH or CₘH₂ₘ₊₁O— (m is an integer from 1 to 5), and
R⁷ to R¹⁰ are each independently —H, —OH, CₙH₂ₙ₊₁O— or CₙH₂ₙ₊₁COO— or 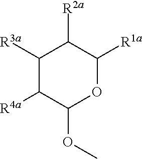
(n is an integer from 1 to 5),
where R^{1a} to R^{4a} are each independently —H, —OH, —CH₂OH,

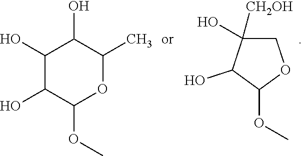

3. The method according to claim 2, wherein
at least one of R¹ to R¹⁰ is —OH.

4. The method according to claim 1, wherein
the biguanide-based compound is selected from the group consisting of metformin, phenformin, buformin and biguanide.

5. The method according to claim 1, wherein
the flavone derivative, hydroxyflavone derivative or flavanone derivative is selected from the group consisting of
2′-hydroxyflavone,
3-hydroxyflavone (flavonol),
3′-hydroxyflavone,
4′-hydroxyflavone,
5-hydroxyflavone (primuliten),
6-hydroxyflavone,
7-hydroxyflavone,
8-hydroxyflavone,
3′,4′-dihydroxyflavone,
3,6-dihydroxyflavone,
3,7-dihydroxyflavone (resogalangin),
4′,7-dihydroxyflavone,
5,7-dihydroxyflavone (chrysin),
7-O-acetyl chrysin (monoacetyl chrysin),
5,7-di-O-methoxy chrysin (dimethyl chrysin),
5,7-di-O-acetyl chrysin (diacetyl chrysin),
6,7-dihydroxyflavone,
7,4′-dihydroxyflavone,
7,8-dihydroxyflavone,
3,5,7-trihydroxyflavone (galangin),
3,7,4′-trihydroxyflavone (resokaempferol),
4′,5,7-trihydroxyflavanone (naringenin),
5,3′,4′-trihydroxyflavone,
5,6,7-trihydroxyflavone (baicalein),
5,7,2′-trihydroxyflavone,
5,7,4′-trihydroxyflavone (apigenin),
5,7,8-trihydroxyflavone (norwogonin),
7,3′,4′-trihydroxyflavone,
7,8,3′-trihydroxyflavone,
7,8,4′-trihydroxyflavone,
4′,5,7-triacetoxy flavone (apigenin triacetate),
5-hydroxy-4′,7-dimethoxyflavone,
5,7-dimethoxy-4′-hydroxyflavone,
5,4′-dimethoxy-7-hydroxyflavone,
3′,4′,5,7-tetrahydroxyflavone (luteolin),
3,4′,5,7-tetrahydroxyflavone (kaempferol),
5,6,7,4′-tetrahydroxyflavone (scutellarein),
4′,5,6,7,8-pentamethoxyflavone (tangeretin),
5,6,7,3′,4′-pentamethoxyflavone (sinensetin),
5,7,8,3′,4′-pentamethoxyflavone (isosinensetin),
3,3′,4′,5,6,7-hexahydroxyflavone (quercetagetin),
3′,4′,5,6,7,8-hexamethoxyflavone (nobiletin),
4′,5,7-trihydroxy-3′-methoxyflavone (chrysoeriol),
5,7,3′-trihydroxy-4′-methoxyflavone (diosmetin),
4′,5,7-trihydroxy-6-methoxyflavone (hispidulin),
5,7,4′-trihydroxy-3,6,3′-trimethoxyflavone (jaceidin),
3′,4′,7-trihydroxy-6-methoxyflavone (nepetin),
3,5,7,3′,4′-pentahydroxy-6-methoxyflavone (patuletin),
3,4′,5,7-tetrahydroxy-3′,6-dimethoxyflavone (spinacetin),
5,7,4′-trihydroxy-3′,5′-dimethoxyflavone (tricin),
7-O-beta-D-apiofuranosyl-1,2-beta-D-glucosyl-5,7,4′-trihydroxyflavone (apiin),
7-O-beta-D-glucosyl-5,7,4′-trihydroxyflavone (apigetrin),
5,7,3′,4′-flavon-3-ol (quercetin),
7,3′,4′-flavon-3-ol (fisetin),
4′,5-dihydroxy-7-methoxyflavone (genkwanin),
4′,5,7-trihydroxyflavanone-7-rhamnoglucoside (naringin),
5-hydroxy-2-(4-hydroxyphenyl)-4-oxo-4H-chromen-7-yl-2-O-(alpha-L-rhamnopyranosyl)-beta-D-glucopyranoside (rhoifoloside), and
8alpha-L-arabinopyranosyl-6beta-D-glucopyranosyl-5,7-dihydroxy-2-(4-hydroxyphenyl)-4H-1-Benzopyran-4-one (schaftoside).

6. The method according to claim 2, wherein
the compound represented by Chemical Formula 5 is selected from the group consisting of
2′-hydroxyflavone,
3-hydroxyflavone (flavonol),
3′-hydroxyflavone,
4′-hydroxyflavone,
5-hydroxyflavone (primuliten),
6-hydroxyflavone,
7-hydroxyflavone,
8-hydroxyflavone,
3′,4′-dihydroxyflavone,
3,6-dihydroxyflavone,
3,7-dihydroxyflavone (resogalangin),
4′,7-dihydroxyflavone,
5,7-dihydroxyflavone (chrysin),
7-O-acetyl chrysin (monoacetyl chrysin),
5,7-di-O-methoxy chrysin (dimethyl chrysin),
5,7-di-O-acetyl chrysin (diacetyl chrysin),
6,7-dihydroxyflavone,
7,4′-dihydroxyflavone,
7,8-dihydroxyflavone,
3,5,7-trihydroxyflavone (galangin),
3,7,4′-trihydroxyflavone (resokaempferol),
4′,5,7-trihydroxyflavanone (naringenin),
5,3′,4′-trihydroxyflavone,
5,6,7-trihydroxyflavone (baicalein),
5,7,2′-trihydroxyflavone,
5,7,4′-trihydroxyflavone (apigenin),
5,7,8-trihydroxyflavone (norwogonin),
7,3′,4′-trihydroxyflavone,
7,8,3′-trihydroxyflavone,
7,8,4′-trihydroxyflavone,
4′,5,7-triacetoxy flavone (apigenin triacetate),
5-hydroxy-4′,7-dimethoxyflavone,
5,7-dimethoxy-4′-hydroxyflavone,
5,4′-dimethoxy-7-hydroxyflavone,
3′,4′,5,7-tetrahydroxyflavone (luteolin),
3,4′,5,7-tetrahydroxyflavone (kaempferol),
5,6,7,4′-tetrahydroxyflavone (scutellarein),
4′,5,6,7,8-pentamethoxyflavone (tangeretin),
5,6,7,3′,4′-pentamethoxyflavone (sinensetin),
5,7,8,3′,4′-pentamethoxyflavone (isosinensetin),
3,3′,4′,5,6,7-hexahydroxyflavone (quercetagetin),
3′,4′,5,6,7,8-hexamethoxyflavone (nobiletin),
4′,5,7-trihydroxy-3′-methoxyflavone (chrysoeriol),
5,7,3′-trihydroxy-4′-methoxyflavone (diosmetin),
4′,5,7-trihydroxy-6-methoxyflavone (hispidulin),
5,7,4′-trihydroxy-3,6,3′-trimethoxyflavone (jaceidin),
3′,4′,7-trihydroxy-6-methoxyflavone (nepetin),
3,5,7,3′,4′-pentahydroxy-6-methoxyflavone (patuletin),
3,4′,5,7-tetrahydroxy-3′,6-dimethoxyflavone (spinacetin),
5,7,4′-trihydroxy-3′,5′-dimethoxyflavone (tricin),
7-O-beta-D-apiofuranosyl-1,2-beta-D-glucosyl-5,7,4′-trihydroxyflavone (apiin),
7-O-beta-D-glucosyl-5,7,4′-trihydroxyflavone (apigetrin),
5,7,3′,4′-flavon-3-ol (quercetin),
7,3′,4′-flavon-3-ol (fisetin),
4′,5-dihydroxy-7-methoxyflavone (genkwanin),
4′,5,7-trihydroxyflavanone-7-rhamnoglucoside (naringin),
5-hydroxy-2-(4-hydroxyphenyl)-4-oxo-4H-chromen-7-yl-2-O-(alpha-L-rhamnopyranosyl)-beta-D-glucopyranoside (rhoifoloside), and
8alpha-L-arabinopyranosyl-6beta-D-glucopyranosyl-5,7-dihydroxy-2-(4-hydroxyphenyl)-4H-1-Benzopyran-4-one (schaftoside).

7. The method according to claim 1, wherein
a blending ratio of the first component including a biguanide-based compound or a pharmaceutically acceptable salt thereof to the second component including a flavone, a hydroxyflavone, a flavanone, a flavone derivative, a hydroxyflavone derivative, a flavanone derivative or a pharmaceutically acceptable salt thereof is from 1:0.0000001 to 1:10 parts by weight.

8. The method according to claim 2, wherein
a blending ratio of the first component including a biguanide-based compound or a pharmaceutically acceptable salt thereof to the second component including a compound represented by Chemical Formula 5 or a pharmaceutically acceptable salt thereof is from 1:0.0000001 to 1:10 parts by weight

9. The method according to claim 1, wherein
the cancer is selected from the group consisting of (A) breast cancers, including (1) ductal carcinoma, including ductal carcinoma in situ (DCIS) (comedocarcinoma, cribriform, papillary, microcapillary), invasive ductal carcinoma (IDC), ductal carcinoma, mucinous (colloidal) carcinoma, papillary carcinoma, metaplastic carcinoma and inflammatory carcinoma; (2) lobular carcinomas, including lobular carcinoma in situ (LCIS) and invasive lobular carcinoma; and (3) Paget's disease of the nipple; (B) cancers of the female reproductive system, including (1) cancers of the cervix, including cervical intraepithelial neoplasia (grade I), cervical intraepithelial neoplasia (grade II), cervical intraepithelial neoplasia (grade III) (squamous cell carcinoma in situ), keratinizing squamous cell carcinoma, nonkeratinizing squamous cell carcinoma, verrucous carcinoma, adenocarcinoma in situ, adenocarcinoma in situ, endometrial type carcinoma, endometrioid adenocarcinoma, clear cell adenocarcinoma, adenoepithelioma, adenoid cystic carcinoma, small cell carcinoma and undifferentiated carcinoma; (2) cancers of the uterine body, including endometrioid carcinoma, adenocarcinoma, adenoacanthoma (adenocarcinoma with squamous metaplasia), adenoepithelioma (mixed adenocarcinoma and squamous cell carcinoma), mucinous adenocarcinoma, serous adenocarcinoma, clear cell adenocarcinoma, squamous cell adenocarcinoma and undifferentiated adenocarcinoma; (3) cancers of the ovary, including serous cystadenoma, serous cystadenoma, mucinous cystadenoma, mucinous cystadenoma, endometrioid tumor, endometrioid adenocarcinoma, clear cell tumor, clear cell cystadenoma and unclassified tumors; (4) cancers of the vagina, including squamous cell carcinoma and adenocarcinoma; and (5) vulvar cancers, including vulvar intraepithelial neoplasia (grade I), vulvar intraepithelial neoplasia (grade II), vulvar intraepithelial neoplasia (grade III) (squamous cell carcinoma in situ); squamous cell carcinoma, verrucous carcinoma, Paget's disease of the vulva, adenocarcinoma (NOS); basal cell carcinoma (NOS) and Bartholin gland carcinoma; (C) cancers of the male reproductive system, including (1) cancer of the penis, including squamous cell carcinoma; (2) cancers of the prostate, including adenocarcinomas, sarcomas, and transitional cell carcinomas of the prostate; and (3) cancers of the testes, including seminoma tumor, non-seminoma tumor, teratomas, embryonic carcinomas, yolk sac tumor and choriocarcinoma; (D) cancers of the heart system, including sarcomas (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma and squamous cell carcinoma of the pharynx; (F) cancers of the lung, including squamous cell carcinoma (epidermoid carcinoma), a variant of squamous cell carcinoma, spindle cell carcinoma, small cell carcinoma, carcinoma of other cells, carcinoma of the intermediate cell type, complex oat cell carcinoma, adenocarcinoma, acinar adenocarcinoma, papillary adenocarcinoma, bronchoalveolar carcinoma, mucin-producing solid carcinoma, giant cell carcinoma, giant cell carcinoma, clear cell carcinoma and sarcoma; (G) cancers of the gastrointestinal tract, including (1) cancers of the ampulla of vater, including primary adenocarcinoma, carcinoid tumor and lymphoma; (2) cancers of the anal canal, including adenocarcinoma, squamous cell carcinoma and melanoma; (3) cancers of the extrahepatic bile duct, including carcinoma in situ, adenocarcinoma, papillary adenocarcinoma, adenocarcinoma, intestinal type, mucinous adenocarcinoma, clear cell adenocarcinoma, signet ring cell carcinoma, adenoepithelioma, squamous cell carcinoma, small cell (oat cell) carcinoma, undifferentiated carcinoma, carcinoma (NOS), sarcoma and carcinoid tumor; (4) cancers of the colon and rectum, including adenocarcinoma in situ, adenocarcinoma, mucinous adenocarcinoma (colloidal type; >50% mucinous carcinoma), signet ring cell carcinoma (greater than 50% of signet ring cells), squamous cell (epidermoid) carcinoma, adenoepithelioma, small cell (oat cell) carcinoma, undifferentiated carcinoma, carcinoma (NOS), sarcoma, lymphoma and carcinoid tumor; (5) cancers of the esophagus, including squamous cell carcinoma, adenocarcinoma, leiomyosarcoma and lymphoma; (6) cancers of the gallbladder, including adenocarcinoma, adenocarcinoma, bowel type, adenoepithelioma, carcinoma in situ, carcinoma (NOS), clear cell adenocarcinoma, mucinous adenocarcinoma, papillary adenocarcinoma, signet ring cell carcinoma, small cell (oat cell) carcinoma, squamous cell carcinoma and undifferentiated carcinoma; (7) cancers of the lips and oral cavity, including squamous cell carcinoma; (8) cancers of the liver, including liver cancer (hepatocellular carcinoma), cholangiocarcinoma, hepatoblastoma, hemangiosarcoma, hepatocellular adenoma and hemangioma; (9) cancers of the exocrine pancreas, including ductal cell carcinoma, giant cell carcinoma multiforme, giant cell carcinoma, osteoclastoid type, adenocarcinoma, adenoepithelioma, mucinous (colloidal) carcinoma, cystadenoma, acinar cell carcinoma, papillary carcinoma, small cell (oat cell) carcinoma, mixed cell type, carcinoma (NOS), undifferentiated carcinoma, endocrine cell tumor arising from islet cells of Langerhans and carcinoid tumor; (10) cancers of the salivary glands, including acinar (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well-differentiated or low grade) and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenoepithelioma, squamous cell carcinoma, small cell carcinoma, undifferentiated carcinoma, lymphoma, sarcoma and carcinoid tumor; and (12) cancers of the small intestine, including adenocarcinoma, lymphoma, carcinoid tumor, Kaposi's sarcoma, leiomyoma, hemangioma, lipoma, neurofibromatosis and fibroma; (H) cancers of the urinary system, including (1) cancers of the kidney, including renal cell carcinoma, carcinoma of the Bellini collecting duct, adenocarcinoma, papillary carcinoma, tubular carcinoma, granular cell tumor, clear cell carcinoma (adenocarcinoma of the kidney), sarcoma of the kidney and nephroblastoma; (2) cancers of the renal pelvis and ureter, including transitional cell carcinoma, papillary transitional cell carcinoma, squamous cell carcinoma and adenocarcinoma; (3) cancers of the urethra, including transitional cell carcinoma, squamous cell carcinoma and adenocarcinoma; and (4) cancers of the bladder, including carcinoma in situ, transitional urothelial cell carcinoma, papillary transitional cell carcinoma, squamous cell carcinoma, adenocarcinoma, and undifferentiated carcinoma; and (I) cancers of muscles, bones and soft tissues, including (1) cancers of the bone, including (a) osteogenesis: osteosarcoma; (b) chondrogenesis: chondrosarcoma and mesenchymal chondrosarcoma; (c) giant cell tumor, malignant; (d) Ewing's sarcoma; (e) vascular tumors: hemangioendothelioma, hemangiopericytoma and hemangiosarcoma; (f) connective tissue tumors: fibrosarcoma, liposarcoma, malignant mesenchymoma and undifferentiated sarcoma; and (g) other tumors: chordoma and adamantinoma of the long bones; (2) cancers of soft tissue, including alveolar soft part sarcoma, angiosarcoma, epithelioid sarcoma, extraosseous chondrosarcoma, fibrosarcoma, leiomyosarcoma, liposarcoma, malignant fibrous histiocytoma, malignant hemangiopericytoma, malignant mesenchymoma, malignant Schwannoma, rhabdomyosarcoma, synovial sarcoma and sarcoma (NOS); (3) cancers of the nervous system, including cancers of the skull (osteoma, hemangioma, granuloma, xanthoma, and osteitis deformans), cancers of the meninges (meningioma, meningiosarcoma, and gliomatosis), cancers of the brain (astrocytoma, meduloblastoma, glioma, ependymal glioma, germinoma (pineal tumor), glioblastoma multiforme, oligodendrocytoma, schwannoma, retinoblastoma, and congenital tumor), and cancers of the spinal cord (neurofibromatosis, meningioma, glioma, sarcoma); (4) hematologic malignancies, including myeloid leukemia (acute and chronic), acute lymphoblastic leukemia, chronic lymphocytic leukemia, myeloproliferative disease, multiple myeloma; myelodysplastic syndrome, Hodgkin's disease and non-Hodgkin's lymphoma (malignant lymphoma); (5) cancers of the endocrine system, including (a) cancers of the thyroid gland, including papillary carcinoma (including those of the follicular region), follicular carcinoma, medullary carcinoma and undifferentiated (anaplastic) carcinoma; and (b) neuroblastomas, including sympathicogonioma, sympathoblastoma, malignant ganglioneuroma, ganglioneuroblastoma and ganglioneuroma; (6) cancers of the skin, including squamous cell carcinoma, spindle cell squamous cell carcinoma, basal cell carcinoma, adenocarcinoma arising from sweat glands or sebaceous glands, and malignant melanoma; and (7) cancers of the eye, including (a) cancer of the conjunctiva, including carcinoma of the conjunctiva; (b) cancers of the eyelid, including basal cell carcinoma, squamous cell carcinoma, melanoma of the eyelid and sebaceous cell carcinoma; (c) cancers of the lacrimal gland, including adenocarcinoma, adenoid cystic carcinoma, carcinoma in pleomorphic adenoma, mucoepidermoid carcinoma and squamous cell carcinoma; (d) cancers of the uvea, including spindle cell melanoma, mixed cell melanoma and epithelioid cell melanoma; (e) cancers of the orbit, including sarcoma of the orbit, soft tissue tumor, and sarcoma of the bone; and (f) retinoblastoma.

10. The method according to claim 1, wherein
the pharmaceutical composition is formulated into a formulation selected from the group consisting of a tablet, a capsule, an injections, a troche, a powder, a granule, a solution, a suspension, an oral solution, an emulsion, a syrup, a suppository, a vaginal tablet and a pill.

11-18. (canceled)

19. The method according to claim 2, wherein
the biguanide-based compound is selected from the group consisting of metformin, phenformin, buformin and biguanide.

20. The method according to claim 2, wherein
the cancer is selected from the group consisting of (A) breast cancers, including (1) ductal carcinoma, including ductal carcinoma in situ (DCIS) (comedocarcinoma, cribriform, papillary, micropapillary), invasive ductal carcinoma (IDC), ductal carcinoma, mucinous (colloidal) carcinoma, papillary carcinoma, metaplastic carcinoma and inflammatory carcinoma; (2) lobular carcinomas, including lobular carcinoma in situ (LCIS) and invasive lobular carcinoma; and (3) Paget's disease of the nipple; (B) cancers of the female reproductive system, including (1) cancers of the cervix, including cervical intraepithelial neoplasia (grade I), cervical intraepithelial neoplasia (grade II), cervical intraepithelial neoplasia (grade III) (squamous cell carcinoma in situ), keratinizing squamous cell carcinoma, nonkeratinizing squamous cell carcinoma, verrucous carcinoma, adenocarcinoma in situ, adenocarcinoma in situ, endometrial type carcinoma, endometrioid adenocarcinoma, clear cell adenocarcinoma, adenoepithelioma, adenoid cystic carcinoma, small cell carcinoma and undifferentiated carcinoma; (2) cancers of the uterine body, including endometrioid carcinoma, adenocarcinoma, adenoacanthoma (adenocarcinoma with squamous metaplasia), adenoepithelioma (mixed adenocarcinoma and squamous cell carcinoma), mucinous adenocarcinoma, serous adenocarcinoma, clear cell adenocarcinoma, squamous cell adenocarcinoma and undifferentiated adenocarcinoma; (3) cancers of the ovary, including serous cystadenoma, serous cystadenoma, mucinous cystadenoma, mucinous cystadenoma, endometrioid tumor, endometrioid adenocarcinoma, clear cell tumor, clear cell cystadenoma and unclassified tumors; (4) cancers of the vagina, including squamous cell carcinoma and adenocarcinoma; and (5) vulvar cancers, including vulvar intraepithelial neoplasia (grade I), vulvar intraepithelial neoplasia (grade II), vulvar intraepithelial neoplasia (grade III) (squamous cell carcinoma in situ); squamous cell carcinoma, verrucous carcinoma, Paget's disease of the vulva, adenocarcinoma (NOS); basal cell carcinoma (NOS) and Bartholin gland carcinoma; (C) cancers of the male reproductive system, including (1) cancer of the penis, including squamous cell carcinoma; (2) cancers of the prostate, including adenocarcinomas, sarcomas, and transitional cell carcinomas of the prostate; and (3) cancers of the testes, including seminoma tumor, non-seminoma tumor, teratomas, embryonic carcinomas, yolk sac tumor and choriocarcinoma; (D) cancers of the heart system, including sarcomas (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma and squamous cell carcinoma of the pharynx; (F) cancers of the lung, including squamous cell carcinoma (epidermoid carcinoma), a variant of squamous cell carcinoma, spindle cell carcinoma, small cell carcinoma, carcinoma of other cells, carcinoma of the intermediate cell type, complex oat cell carcinoma, adenocarcinoma, acinar adenocarcinoma, papillary adenocarcinoma, bronchoalveolar carcinoma, mucin-producing solid carcinoma, giant cell carcinoma, giant cell carcinoma, clear cell carcinoma and sarcoma; (G) cancers of the gastrointestinal tract, including (1) cancers of the ampulla of vater, including primary adenocarcinoma, carcinoid tumor and lymphoma; (2) cancers of the anal canal, including adenocarcinoma, squamous cell carcinoma and melanoma; (3) cancers of the extrahepatic bile duct, including carcinoma in situ, adenocarcinoma, papillary adenocarcinoma, adenocarcinoma, intestinal type, mucinous adenocarcinoma, clear cell adenocarcinoma, signet ring cell carcinoma, adenoepithelioma, squamous cell carcinoma, small cell (oat cell) carcinoma, undifferentiated carcinoma, carcinoma (NOS), sarcoma and carcinoid tumor; (4) cancers of the colon and rectum, including adenocarcinoma in situ, adenocarcinoma, mucinous adenocarcinoma (colloidal type; >50% mucinous carcinoma), signet ring cell carcinoma (greater than 50% of signet ring cells), squamous cell (epidermoid) carcinoma, adenoepithelioma, small cell (oat cell) carcinoma, undifferentiated carcinoma, carcinoma (NOS), sarcoma, lymphoma and carcinoid tumor; (5) cancers of the esophagus, including squamous cell carcinoma, adenocarcinoma, leiomyosarcoma and lymphoma; (6) cancers of the gallbladder, including adenocarcinoma, adenocarcinoma, bowel type, adenoepithelioma, carcinoma in situ, carcinoma (NOS), clear cell adenocarcinoma, mucinous adenocarcinoma, papillary adenocarcinoma, signet ring cell carcinoma, small cell (oat cell) carcinoma, squamous cell carcinoma and undifferentiated carcinoma; (7) cancers of the lips and oral cavity, including squamous cell carcinoma; (8) cancers of the liver, including liver cancer (hepatocellular carcinoma), cholangiocarcinoma, hepatoblastoma, hemangiosarcoma, hepatocellular adenoma and hemangioma; (9) cancers of the exocrine pancreas, including ductal cell carcinoma, giant cell carcinoma multiforme, giant cell carcinoma, osteoclastoid type, adenocarcinoma, adenoepithelioma, mucinous (colloidal) carcinoma, cystadenoma, acinar cell carcinoma, papillary carcinoma, small cell (oat cell) carcinoma, mixed cell type, carcinoma (NOS), undifferentiated carcinoma, endocrine cell tumor arising from islet cells of Langerhans and carcinoid tumor; (10) cancers of the salivary glands, including acinar (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well-differentiated or low grade) and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenoepithelioma, squamous cell carcinoma, small cell carcinoma, undifferentiated carcinoma, lymphoma, sarcoma and carcinoid tumor; and (12) cancers of the small intestine, including adenocarcinoma, lymphoma, carcinoid tumor, Kaposi's sarcoma, leiomyoma, hemangioma, lipoma, neurofibromatosis and fibroma; (H) cancers of the urinary system, including (1) cancers of the kidney, including renal cell carcinoma, carcinoma of the Bellini collecting duct, adenocarcinoma, papillary carcinoma, tubular carcinoma, granular cell tumor, clear cell carcinoma (adenocarcinoma of the kidney), sarcoma of the kidney and nephroblastoma; (2) cancers of the renal pelvis and ureter, including transitional cell carcinoma, papillary transitional cell carcinoma, squamous cell carcinoma and adenocarcinoma; (3) cancers of the urethra, including transitional cell carcinoma, squamous cell carcinoma and adenocarcinoma; and (4) cancers of the bladder, including carcinoma in situ, transitional urothelial cell carcinoma, papillary transitional cell carcinoma, squamous cell carcinoma, adenocarcinoma, and undifferentiated carcinoma; and (I) cancers of muscles, bones and soft tissues, including (1) cancers of the bone, including (a) osteogenesis: osteosarcoma; (b) chondrogenesis: chondrosarcoma and mesenchymal chondrosarcoma; (c) giant cell tumor, malignant; (d) Ewing's sarcoma; (e) vascular tumors: hemangioendothelioma, hemangiopericytoma and hemangiosarcoma; (f) connective tissue tumors: fibrosarcoma, liposarcoma, malignant mesenchymoma and undifferentiated sarcoma; and (g) other tumors: chordoma and adamantinoma of the long bones; (2) cancers of soft tissue, including alveolar soft part sarcoma, angiosarcoma, epithelioid sarcoma, extraosseous chondrosarcoma, fibrosarcoma, leiomyosarcoma, liposarcoma, malignant fibrous histiocytoma, malignant hemangiopericytoma, malignant mesenchymoma, malignant Schwannoma, rhabdomyosarcoma, synovial sarcoma and sarcoma (NOS); (3) cancers of the nervous system, including cancers of the skull (osteoma, hemangioma, granuloma, xanthoma, and osteitis deformans), cancers of the meninges (meningioma, meningiosarcoma, and gliomatosis), cancers of the brain (astrocytoma, meduloblastoma, glioma, ependymal glioma, germinoma (pineal tumor), glioblastoma multiforme, oligodendrocytoma, schwannoma, retinoblastoma, and congenital tumor), and cancers of the spinal cord (neurofibromatosis, meningioma, glioma, sarcoma); (4) hematologic malignancies, including myeloid leukemia (acute and chronic), acute lymphoblastic leukemia, chronic lymphocytic leukemia, myeloproliferative disease, multiple myeloma; myelodysplastic syndrome, Hodgkin's disease and non-Hodgkin's lymphoma (malignant lymphoma); (5) cancers of the endocrine system, including (a) cancers of the thyroid gland, including papillary carcinoma (including those of the follicular region), follicular carcinoma, medullary carcinoma and undifferentiated (anaplastic) carcinoma; and (b) neuroblastomas, including sympathicogonioma, sympathoblastoma, malignant ganglioneuroma, ganglioneuroblastoma and ganglioneuroma; (6) cancers of the skin, including squamous cell carcinoma, spindle cell squamous cell carcinoma, basal cell carcinoma, adenocarcinoma arising from sweat glands or sebaceous glands, and malignant melanoma; and (7) cancers of the eye, including (a) cancer of the conjunctiva, including carcinoma of the conjunctiva; (b) cancers of the eyelid, including basal cell carcinoma, squamous cell carcinoma, melanoma of the eyelid and sebaceous cell carcinoma; (c) cancers of the lacrimal gland, including adenocarcinoma, adenoid cystic carcinoma, carcinoma in pleomorphic adenoma, mucoepidermoid carcinoma and squamous cell carcinoma; (d) cancers of the uvea, including spindle cell melanoma, mixed cell melanoma and epithelioid cell melanoma; (e) cancers of the orbit, including sarcoma of the orbit, soft tissue tumor, and sarcoma of the bone; and (f) retinoblastoma.

21. The method according to claim 2, wherein
the pharmaceutical composition is formulated into a formulation selected from the group consisting of a tablet, a capsule, an injections, a troche, a powder, a granule, a solution, a suspension, an oral solution, an emulsion, a syrup, a suppository, a vaginal tablet and a pill.
